(19) United States
(12) Patent Application Publication
(10) Pub. No.: US 2026/0335250 A1
(43) Pub. Date: Oct. 29, 2026

(54) IONIZABLE CATIONIC COMPOUND

(71) Applicant: SEQIRUS INC., Holly Springs, NC (US)

(72) Inventors: Ching Kim TYE, Holly Springs, NC (US); Eric L. DANE, Holly Springs, NC (US); Cangjie YANG, Holly Springs, NC (US)

(21) Appl. No.: 19/154,455

(22) PCT Filed: Feb. 6, 2024
(86) PCT No.: PCT/IB2024/051050

§ 371 Date: Aug. 7, 2025

Related U.S. Application Data
(60) Provisional application No. 63/484,025, filed on Feb. 9, 2023.

(51) Int. Cl. A61K 9/51; A61K 31/7105; A61K 39/00; C07C 217/28; C07C 323/25

(52) U.S. Cl. CPC A61K 9/5123; A61K 31/7105; C07C 217/28; C07C 323/25; A61K 2039/53; A61K 2039/55555; A61K 2039/6018

(57) ABSTRACT

Novel ionizable lipid compounds of Formula I are provided. The use of the compounds in forming lipid nanoparticles is described. The lipid nanoparticles may encapsulate a therapeutic, such as a nucleic acid, and these may be used in the delivery of the therapeutic and in methods of treating certain conditions or for inducing an immune response.

### FIELD

The present disclosure relates to cationic and/or ionizable lipid compounds that can be used, in combination with other lipid molecules, to form lipid nanoparticles for delivery of a polynucleotide to a subject.

### BACKGROUND

Nucleic acid-based therapies have shown substantial promise in a range of therapeutic applications. The delivery of polynucleotides such as messenger RNA (mRNA), small interfering RNA (siRNA), antisense oligonucleotides, plasmids, DNA and the like does, however, present a number of challenges. Free nucleic acids, such as RNAs, are subject to rapid enzymatic degradation and so generally do not persist systemically. Additionally, due to their negative charge the nucleic acids may not be able to effectively cross the cellular barriers to enter the necessary intracellular compartment, for example, for translation or to otherwise achieve their effect.

Lipid particles, such as lipid nanoparticles (LNPs), have therefore been used to formulate nucleic acids so as to protect them from degradation and improve cellular uptake and intracellular delivery. LNPs are commonly formed from ionizable cationic lipids and other lipid components such as neutral lipids, sterols such as cholesterol and PEGylated lipids. Ionizable cationic lipids are amphiphilic molecules having a lipophilic region containing one or more hydrocarbon groups and a hydrophilic region containing at least one positively charged or ionizable polar head group. Such cationic lipids are ionized at an appropriate pH and can then form a positively charged complex with nucleic acids, making it easier for the nucleic acids to pass through the plasma membrane of the cell and enter the cytoplasm.

The first siRNA therapeutic to be approved, Onpattro (patisiran), entered the market just a few years ago for treatment of hereditary amyloidogenic transthyretin (TTR) amyloidosis. Patisiran's therapeutic effect relies on siRNA-mediated TTR gene silencing, preventing mutant protein production to at least prevent disease progression. The efficient delivery of the siRNA depends upon the LNP technology. Even more recently, nucleic acid vaccines have emerged as a promising approach to the treatment and prevention of various diseases, including against the severe acute respiratory syndrome coronavirus 2 (SARS-CoV-2) responsible for causing the on-going worldwide pandemic of the severely infectious coronavirus disease 2019 (COVID-19). mRNA vaccines rely on the delivery of the mRNA into the cytoplasm of host cells, where it is transcribed into antigenic proteins to trigger the production of an immune response. The large size and negative charge of mRNA prevents cellular uptake and so LNPs are again necessary for appropriate delivery.

Different ionizable cationic lipids may present not only different physicochemical profiles, including their acid dissociation constant (pKa) value, thereby affecting their ability to complex with the nucleic acid, but also different toxicity profiles in vivo and so it will be apparent to the skilled person that there is an on-going need for improved ionizable cationic lipid compounds which are suitable to form lipid particles, such as lipid LNPs, for delivery of nucleic acids and polynucleotides.

### BRIEF SUMMARY

Embodiments of the present invention provide for novel ionizable cationic lipid compounds, and pharmaceutically acceptable salts, prodrugs and stereoisomers thereof, which can form lipid particles, for example LNPs, in the presence of additional lipids including one or more of neutral lipids, charged lipids, structural lipids, PEGylated lipids and analogs thereof, and which can be used for the delivery of a polynucleotide. Compositions comprising such lipid particles, methods of forming the lipid particles, their use in delivering a polynucleotide and methods of using the lipid particles in the treatment of a range of diseases, disorders and conditions are provided.

Accordingly, in one embodiment, the present disclosure provides for a compound of Formula I.

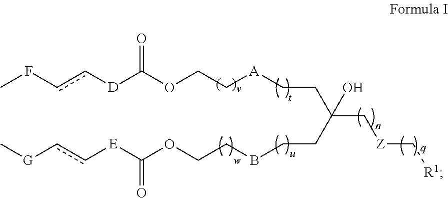

or a pharmaceutically acceptable salt, prodrug or stereoisomer thereof, wherein:
R¹ is selected from the group consisting of Formulae I-A to I-I:

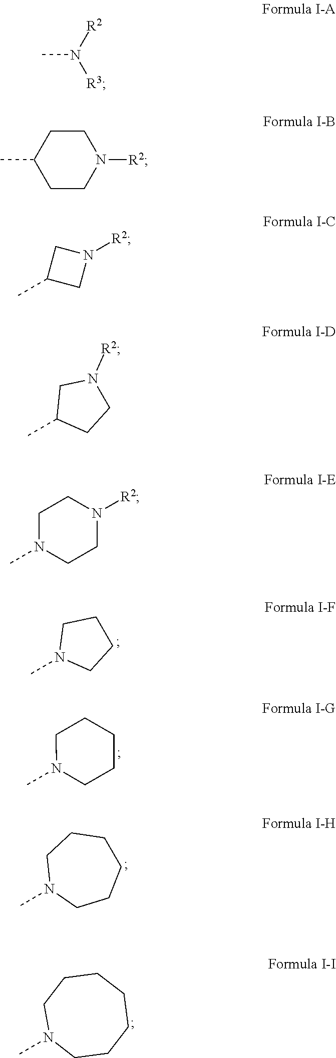

Z, A, and B are each independently selected from the group consisting of —C(H)₂—, —N(H)—, —O—, and —S—; wherein at least one of Z, A, and B is —N(H)—, —O—, or —S—; n and q are each independently an integer from 0 to 8; t, u, v, and w are each independently an integer from 0 to 10; D, E, F, and G are each independently a linear or branched C₁₋₁₆-alkyl; [symbol] represents a single or double bond; and R² and R³ are each independently selected from the group consisting of C₁₋₈-alkyl and C₁₋₈-alkanol.

In embodiments, lipid nanoparticles (LNPs) are provided which comprise a compound of Formula I.

In embodiments, the LNPs further comprise a polynucleotide.

In embodiments, a pharmaceutical composition comprising such LNPs and at least one pharmaceutically acceptable carrier, diluent or excipient is provided.

In other embodiments, a method of forming an LNP comprising a compound of Formula I is provided.

In additional embodiments, a method of delivering a polynucleotide, within the foregoing LNPs, to a cell is provided.

In further embodiments, a method of producing a polypeptide of interest in a cell is provided.

In still further embodiments, a method of treating a disease, disorder or condition in a subject is provided by administering one or more of the foregoing LNPs comprising a polynucleotide, or a pharmaceutical composition comprising same, to a subject in need of such treatment.

The LNPs comprising a polynucleotide, or a pharmaceutical composition comprising same, may be delivered to the subject as a component of a vaccine.

### DETAILED DESCRIPTION

The present disclosure is based on the use of certain novel biodegradable lipid compounds as a component of an LNP for the delivery of a polynucleotide. The novel lipid compounds present biodegradable groups which may assist in reducing toxicity or improving clearance, in vivo.

In one embodiment, the LNP may be a component of a vaccine although the therapeutic use of the compounds described herein and the use of LNPs which they form a component of, is not so limited.

In some embodiments the LNPs formed may be suitable for the delivery of messenger RNA (mRNA).

In some embodiments, the LNPs formed may be suitable for the delivery of mRNA as a component of a mRNA vaccine.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the present disclosure.

Any embodiment of the present disclosure herein shall be taken to apply mutatis mutandis to any other embodiment of the disclosure unless specifically stated otherwise.

Unless specifically defined otherwise, all technical and scientific terms used herein shall be taken to have the same meaning as commonly understood by one of ordinary skill in the art (for embodiments, in organic synthetic chemistry, cell culture, molecular genetics, immunology, immunohistochemistry, protein chemistry, and biochemistry).

Unless otherwise indicated, any recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The terms “from” and “to”, when indicating a range, shall be understood to mean the range is inclusive of the recited lower and upper values. For example, “p is an integer from 0 to 6” shall be understood as including the situation in which p is not present (p is 0), that in which p is 6, as well as each whole number integer value in between, i.e. p is 1, 2, 3, 4, or 5.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### SELECTED DEFINITIONS

As used herein, the terms “lipid particle”, “lipid nanoparticle” or “LNP” shall be understood to refer to lipid-based particles having at least one dimension on the order of nanometers (e.g., 1-1,000 nm) and which comprises a compound of any formulae described herein. In embodiments, LNPs are formulated in a composition for delivery of a polynucleotide to a desired target such as a cell, tissue, organ, tumor, and the like. The LNPs generally comprise an ionizable cationic compound of the present disclosure and one or more of a neutral lipid, charged lipid, sterol and PEGylated lipid. In embodiments, the lipid particle or LNP may be selected from liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles. In embodiments, the lipid nanoparticle or LNP may have a structure that includes a single monolayer or bilayer of lipids that encapsulates a solid phase. In preferred embodiments, the lipid nanoparticle or LNP does not have an aqueous phase or other liquid phase in its interior.

A “cationic compound”, “ionizable cationic compound”, “cationic lipid compound”, “ionizable cationic lipid compound”, or like terms, refer to a lipid compound of any structural formulae described herein and which is capable of bearing a positive charge. Ionizable cationic lipids disclosed herein include one or more nitrogen-containing groups which may bear the positive charge. They are ionizable such that they can exist in a positively charged or neutral form, depending on pH. The ionization of the cationic lipid affects the surface charge of the lipid nanoparticle under different pH conditions.

The term “neutral lipid” refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and phophatidylethanolamines such as 1,2-Dioleoyl-sn-glycero-3-phosphoethanolamine (DOPE), sphingomyelins (SM).

The term “charged lipid” refers to any of a number of lipid species that exist in either a positively charged or negatively charged form independent of the pH within a useful physiological range e.g. pH ~3 to pH ~9. Non-limiting examples of charged lipids include phosphatidylserines, phosphatidic acids, phosphatidylglycerols, phosphatidylmositols, sterol hemisuccinates, dialkyl trimethylammonium-propanes, (including DOTAP and DOTMA), dialkyl dimethylaminopropanes, ethyl phosphocholines, and dimethylaminoethane carbamoyl sterols.

The term “polynucleotide” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), self-amplifying messenger RNA (saRNA or samRNA), antisense RNA, miRNA, micRNA, multivalent RNA, dicer substrate RNA or viral RNA (vRNA), and combinations thereof. Polynucleotides include those containing known nucleotide analogs or modified backbone residues or linkages, which are synthetic, naturally occurring, and non-naturally occurring, and which have similar binding properties as the reference polynucleotide. Examples of such analogs include, without limitation, phosphorothioates, phosphoramidates, methyl phosphonates, chiral-methyl phosphonates, 2-O-methyl ribonucleotides, and peptide-nucleic acids (PNAs). Unless specifically limited, the term encompasses polynucleotides containing known analogues of natural nucleotides that have similar binding properties as the reference polynucleotide. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

An “effective amount” or “therapeutically effective amount” of a therapeutic polynucleotide is an amount sufficient to produce the desired effect, such as an increase or inhibition of expression of a target sequence in comparison to the normal expression level detected in the absence of the polynucleotide. Suitable assays for measuring expression of a target gene or target sequence include, examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays.

As used herein, “prodrug” is meant to indicate a compound that may be converted under physiological conditions or by solvolysis to a compound of any one or more of the formulae described herein. Thus, the term “prodrug” refers to a metabolic precursor of such a compound that is pharmaceutically acceptable. A prodrug may be inactive when administered to a subject in need thereof, but is converted in vivo to an active form. The term may also include any covalently bonded carriers, which release the active compound in vivo when such prodrug is administered to a mammalian subject. Prodrugs of a compound of formula I, or other formulae described herein, may be prepared by modifying functional groups present in the compound in such a way that the modifications are cleaved, either in routine manipulation or in vivo, to the parent compound.

“Pharmaceutically acceptable carrier, diluent or excipient”, or like terms, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxy toluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

“Pharmaceutically acceptable salt” includes both acid and base addition salts. Lists of suitable salts may be found in Remington's Pharmaceutical Sciences, 18th ed., Mack Publishing Company, Easton, PA, 1990, p. 1445, and Journal of Pharmaceutical Science, 66, 2-19 (1977). Acid addition salts are those which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like. Base addition salts are those which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like.

As used herein, “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

As used herein, the term “biodegradable group” is a group that may facilitate faster metabolism of a lipid in a mammal. An ester is a suitable biodegradable group and compounds of the present disclosure present two such groups for improved in vivo biodegradability.

As used herein, “encapsulation efficiency” refers to the amount of a polynucleotide that becomes part of an LNP composition, relative to the initial total amount of polynucleotide used in the preparation of the LNP composition. For example, if 92 mg of polynucleotides are encapsulated in an LNP composition out of a total 100 mg of polynucleotide initially provided to the composition, the encapsulation efficiency may be given as 92%. As used herein, “encapsulation” may refer to complete, substantial, or partial enclosure, confinement, surrounding, or encasement.

As used herein, the term “subject” shall be taken to mean any animal, such as a mammal, and including humans. Exemplary subjects include but are not limited to humans and non-human primates. For example, the subject is a human.

As used herein, the term “mammal” includes humans and both domestic animals such as laboratory animals and household pets (e.g., cats, dogs, swine, cattle, sheep, goats, horses, rabbits), and non-domestic animals such as wildlife and the like.

As used herein, the term “alkyl” encompasses both straight chain (i.e., linear) and branched chain hydrocarbon groups. Examples of alkyl groups include methyl, ethyl, n-propyl, iso-propyl, n-butyl, t-butyl, i-butyl, sec-butyl, pentyl, and hexyl groups. In one example, the alkyl group is of one to sixteen carbon atoms (i.e. C₁₋₁₆alkyl).

As used herein, the term “alkanol” encompasses both straight chain (i.e., linear) and branched chain hydrocarbon groups to which an —OH (alcohol) group is substituted. Examples of alkanol groups include methanol, ethanol, and propanol groups. In one example, the alkanol group is of one to five carbon atoms (i.e., C₁₋₅alkanol).

### Compounds

In embodiments, the compounds of the present disclosure may provide for advantages over other select prior art ionizable cationic lipid compounds including one or more of: improved complexation with a polynucleotide; beneficial pKa properties; improved encapsulation efficiency as part of an LNP; reduced toxicity; improved biodegradability; improved in vivo clearance; desirable N:P ratio when complexed with a polynucleotide; desirable polydispersity index for the LNPs comprising them; and improved LNP formation.

In one broad form, the present disclosure provides for a compound of Formula I:

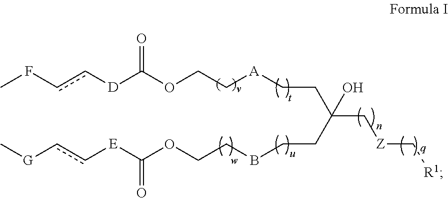

or a pharmaceutically acceptable salt, prodrug or stereoisomer thereof, wherein:
R¹ is selected from the group consisting of Formulae I-A to I-I:

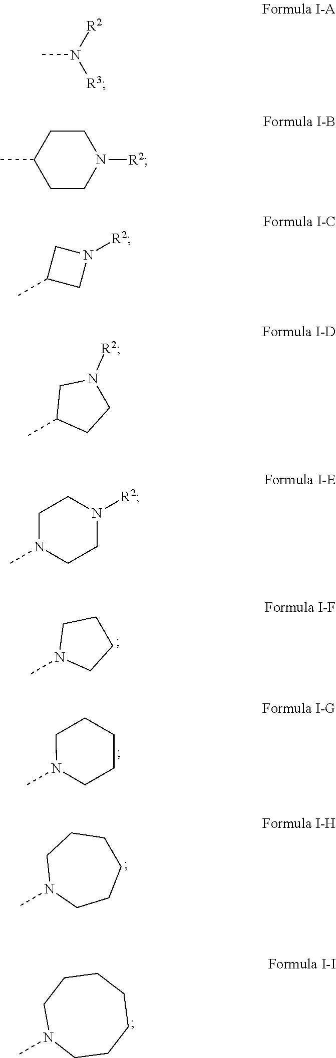

Z, A, and B are each independently selected from the group consisting of —C(H)₂—, —N(H)—, —O—, and —S—; wherein at least one of Z, A, and B is —N(H)—, —O—, or —S—;
n and q are each independently an integer from 0 to 8;
t, u, v, and w are each independently an integer from 0 to 10;
D, E, F, and G are each independently a linear or branched C₁₋₁₆-alkyl;
[symbol] represents a single or double bond; and
R² and R³ are each independently selected from the group consisting of C₁₋₈-alkyl and C₁₋₈-alkanol.

In the compound of Formula I, R¹ is selected from the group consisting of Formulae I-A to I-I:

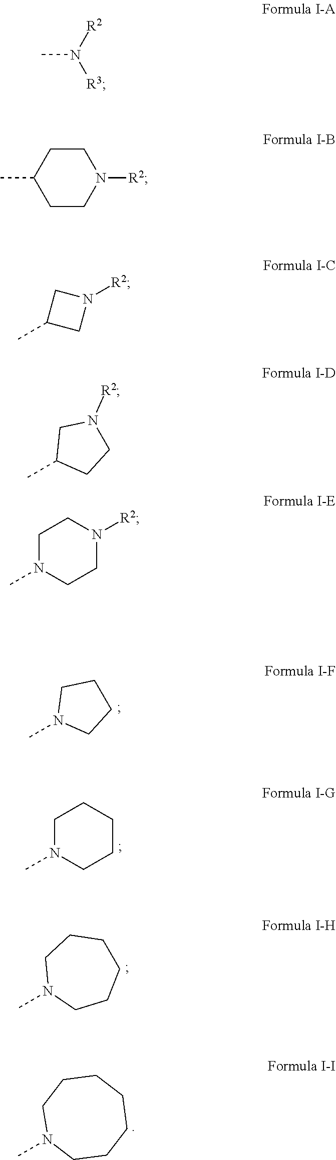

Accordingly, in embodiments, in the compound of Formula I, R¹ is a group of Formula I-A:

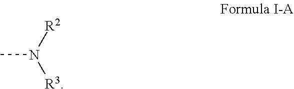

In other embodiments, in the compound of Formula I, R¹ is a group of Formula I-B:

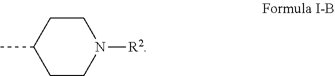

In other embodiments, in the compound of Formula I, R¹ is a group of Formula I-C:

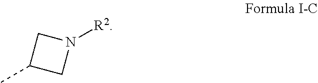

In other embodiments, in the compound of Formula I, R¹ is a group of Formula I-D:

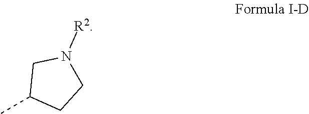

In other embodiments, in the compound of Formula I, R¹ is a group of Formula I-E:

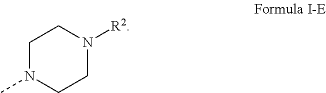

In other embodiments, in the compound of Formula I, R¹ is a group of Formula I-F:

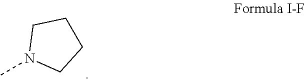

In other embodiments, in the compound of Formula I, R¹ is a group of Formula I-G:

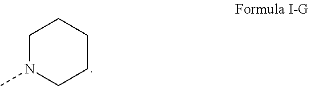

In other embodiments, in the compound of Formula I, R¹ is a group of Formula I-H:

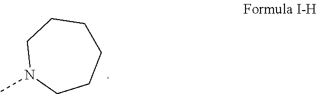

In other embodiments, in the compound of Formula I, R¹ is a group of Formula I-I:

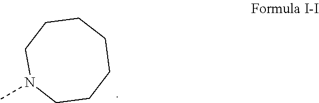

In the compound of Formula I, Z, A, and B are each independently selected from the group consisting of —C(H)₂—, —N(H)—, —O—, and —S—.

In embodiments, Z is independently selected from the group consisting of —C(H)₂—, —N(H)—, —O—, and —S—. In embodiments, Z is —C(H)₂—. In embodiments, Z is —N(H)—. In embodiments, Z is —O—. In embodiments, Z is —S—. In an embodiment, Z is selected from the group consisting of —N(H)—, —O—, and —S—.

In embodiments, A is independently selected from the group consisting of —C(H)₂—, —N(H)—, —O—, and —S—. In embodiments, A is selected from the group consisting of —N(H)—, —O—, and —S—. In embodiments, A is —C(H)₂—. In embodiments, A is —N(H)—. In embodiments, A is —O—. In embodiments, A is —S—.

In embodiments, B is independently selected from the group consisting of —C(H)₂—, —N(H)—, —O—, and —S—. In embodiments, B is selected from the group consisting of —N(H)—, —O—, and —S—. In embodiments, B is —C(H)₂—. In embodiments, B is —N(H)—. In embodiments, B is —O—. In embodiments, B is —S—.

It will be understood that Z, A, and B are independently selected from one another. Accordingly, one or more of Z, A, and B may be the same (e.g., Z, A, and B are —S—; or Z is —S—, A is —S—, and B is —O—), or alternatively, may each be different (e.g., Z is —O—, A is —S—, and B is —N(H)—).

In the compound of Formula I, at least one of Z, A, and B is —N(H)—, —O—, or —S—. That is, each of Z, A, and B cannot simultaneously be —C(H)₂—. That is, the compound of Formula I cannot be:

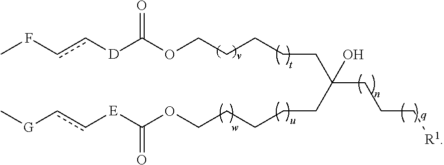

In the compound of Formula I, n and q are each independently an integer from 0 to 8. That is, in embodiments, n and q are each independently an integer selected from the group consisting of 0, 1, 2, 3, 4, 5, 6, 7, and 8. In embodiments, n is an integer independently selected from the group consisting of 0, 1, 2, 3, 4, 5, 6, 7, and 8. In embodiments, q is an integer independently selected from the group consisting of 0, 1, 2, 3, 4, 5, 6, 7, and 8. The integer determines the carbon length encompassed by the relevant parentheses. In embodiments, n and q are each independently an integer from 0 to 4 (i.e., 0, 1, 2, 3, or 4). In embodiments, n is 2. In embodiments, q is 2. In embodiments, n is 2 and q is 2.

It will be understood that n and q are independently selected from one another. Accordingly, n and q may be the same integer (e.g., n is 2 and q is 2), or alternatively, may each be a different integer (e.g., n is 2 and q is 3).

In the instance where n is 0, it will be understood that the carbon chain encompassed in the relevant parentheses is absent. Instead, the adjacent carbon atom is directly bonded to the adjacent Z group. Similarly, in the instance where q is 0, it will be understood that the carbon chain encompassed in the relevant parentheses is absent. Instead, the adjacent Z group is directly bonded to the adjacent R¹ group.

In the instance where n or q is an integer greater than 1, and thus a carbon chain of at least two integers (i.e., at least —C(H)₂—C(H)₂—) is present, the carbon chain is linear in structure (i.e., linear C₂-alkyl). For example, when n or q is 3, the carbon chain will have a structure of —C(H)₂—C(H)₂—C(H)₂— (i.e., linear C₃-alkyl). Similarly, for example, when n or q is 4, the carbon chain will have a structure of —C(H)₂—C(H)₂—C(H)₂—C(H)₂— (i.e., linear C₄-alkyl). That is, there is no branching of the carbon chain when n or q is greater than 1.

In the compound of Formula I, t, u, v, and w are each independently an integer from 0 to 10. That is, in embodiments, t, y, v, and w are each independently an integer selected from the group consisting of 0, 1, 2, 3, 4, 5, 6, 7, 8, 9, and 10. In embodiments, t is an integer independently selected from the group consisting of 0, 1, 2, 3, 4, 5, 6, 7, 8, 9, and 10. In embodiments, u is an integer independently selected from the group consisting of 0, 1, 2, 3, 4, 5, 6, 7, 8, 9, and 10. In embodiments, v is an integer independently selected from the group consisting of 0, 1, 2, 3, 4, 5, 6, 7, 8, 9, and 10. In embodiments, w is an integer independently selected from the group consisting of 0, 1, 2, 3, 4, 5, 6, 7, 8, 9, and 10. The integer determines the carbon length encompassed by the relevant parentheses.

It will be understood that t, u, v, and w are independently selected from one another. Accordingly, one or more of t, u, v, and w may be the same integer (e.g., t is 2, u is 2, v is 2, and w is 2), or alternatively, may each be a different integer (e.g., t is 2, u is 3, v is 4, and w is 5).

In the instance where t is 0, it will be understood that the carbon chain encompassed in the relevant parentheses is absent. Instead, the adjacent carbon atom is directly bonded to the adjacent A group. Similarly, in the instance where u is 0, it will be understood that the carbon chain encompassed in the relevant parentheses is absent. Instead, the adjacent carbon atom is directly bonded to the B group. In the instance where v is 0, it will be understood that the carbon chain encompassed in the relevant parentheses is absent. Instead, the adjacent carbon atom is directly bonded to the A group. In the instance where w is 0, it will be understood that the carbon chain encompassed in the relevant parentheses is absent. Instead, the adjacent carbon atom is directly bonded to the B group.

In the instance where t, u, v, or w is an integer greater than 1, and thus a carbon chain of at least two integers (i.e., at least —C(H)₂—C(H)₂—) is present, the carbon chain is linear in structure (i.e., linear C₂-alkyl), as described above for n and q.

In the compound of Formula I, D, E, F, and G are each independently a linear or branched C₁₋₁₆-alkyl. In embodiments, D is a linear or branched C₁₋₁₆-alkyl. In embodiments, E is a linear or branched C₁₋₁₆-alkyl. In embodiments, E is a linear or branched C₁₋₁₆-alkyl. In embodiments, F is a linear or branched C₁₋₁₆-alkyl. That is, D, E, F, and G may each independently be a C₁-alkyl, a C₂-alkyl, a C₃-alkyl, a C₄-alkyl, and so forth.

It will be understood that D, E, F, and G are independently selected from one another. Accordingly, one or more of D, E, F, and G may be the same linear or branched C₁₋₁₆-alkyl group, or alternatively, may each be a different linear or branched C₁₋₁₆-alkyl group. In embodiments, D, E, F, and G are each independently a linear C₁₋₁₆-alkyl. In embodiments, D, E, F, and G are each independently a linear C₁₋₈-alkyl. In embodiments, at least one of D, E, F, and G is a branched C₁₋₁₆-alkyl. In embodiments, at least one of D, E, F, and G is a branched C₁₋₈-alkyl.

In the instance where any of D, E, F, and G is a branched C₁₋₁₆-alkyl group, it will be understood that at least one carbon chain will continue in the structure so as to be bonded to the adjacent atoms/groups as depicted in the structure of Formula I.

In the compound of Formula I, [symbol] represents a single or double bond. Each depiction of [symbol] in the compound of Formula I is independent of each other, meaning the compound of Formula I may contain 0, 1, or 2 double bonds. In embodiments, [symbol] is a single bond. In embodiments, [symbol] is a double bond.

In the compound of Formula I, and particularly Formula I-A and Formula I-B, R² and R³ are each independently selected from the group consisting of C₁₋₈-alkyl and C₁₋₈-alkanol. In embodiments, R² is C₁₋₈-alkyl. In embodiments, R² is C₁₋₈-alkanol. In embodiments, R³ is C₁₋₈-alkyl. In embodiments, R³ is C₁₋₈-alkanol.

It will be understood that R² and R³ are independently selected from one another. Accordingly, R² and R³ may each be the same linear or branched C₁₋₈-alkyl group or linear or branched C₁₋₈-alkanol group, or alternatively, may each be a different linear or branched C₁₋₈-alkyl group or linear or branched C₁₋₈-alkanol group.

In embodiments, R² is C₁₋₈-alkyl. In embodiments, R² is C₁₋₃-alkyl. In an embodiment, R² is C₁-alkyl. In an embodiment, R² is C₂-alkyl. In an embodiment, R² is C₃-alkyl.

In instances where R¹ is Formula I-A, and R³ is present, R³ is C₁₋₈-alkyl. In embodiments, R³ is C₁₋₃-alkyl. In an embodiment, R³ is C₁-alkyl. In an embodiment, R³ is C₂-alkyl. In an embodiment, R³ is C₃-alkyl.

As discussed above, in embodiments, R¹ is selected from the group consisting of Formula I-A and Formula I-B.

In embodiments, Formula I-A is selected from the group consisting of

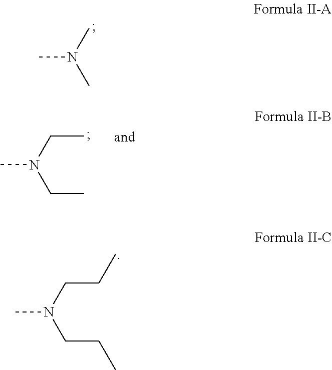

In an embodiment, Formula I-A is Formula II-A. In an embodiment, Formula I-A is Formula II-B. In an embodiment, Formula I-A is Formula II-C.

In embodiments, Formula I-B is selected from the group consisting of:

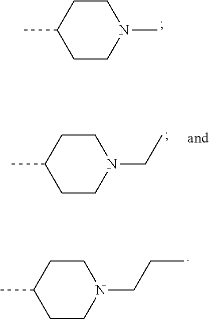

In an embodiment, Formula I-B is Formula II-D. In an embodiment, Formula I-B is formula II-E. In an embodiment, Formula I-B is formula II-F.

In any embodiment of Formula I, A and B may be the same. For example, A and B may be selected from the group consisting of —C(H)₂—, —O—, and —S—. Z may be selected from the groups previously described independently of A and B.

In certain embodiments, when A and B are the same, Z may be selected from —C(H)₂—, and —O—.

In embodiments, the compound of Formula I is a compound of Formula III-A:

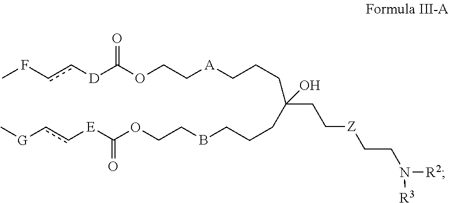

wherein R², R³, Z, A, B, D, E, F, and G are as defined herein.

In embodiments, the compound of Formula I is a compound of Formula IV-A:

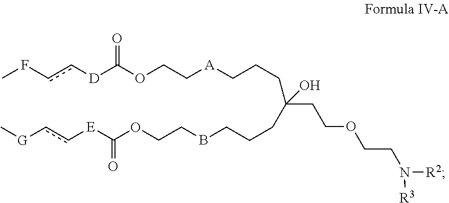

wherein R², R³, A, B, D, E, F, and G are as defined herein.

In embodiments, the compound of Formula I is a compound of Formula V-A:

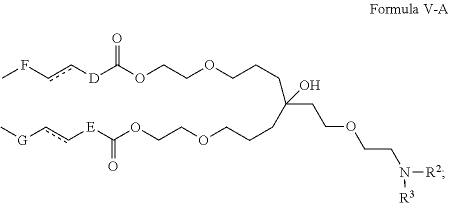

wherein R², R³, D, E, F, and G are as defined herein.

In embodiments, the compound of Formula I is selected from the group consisting of:

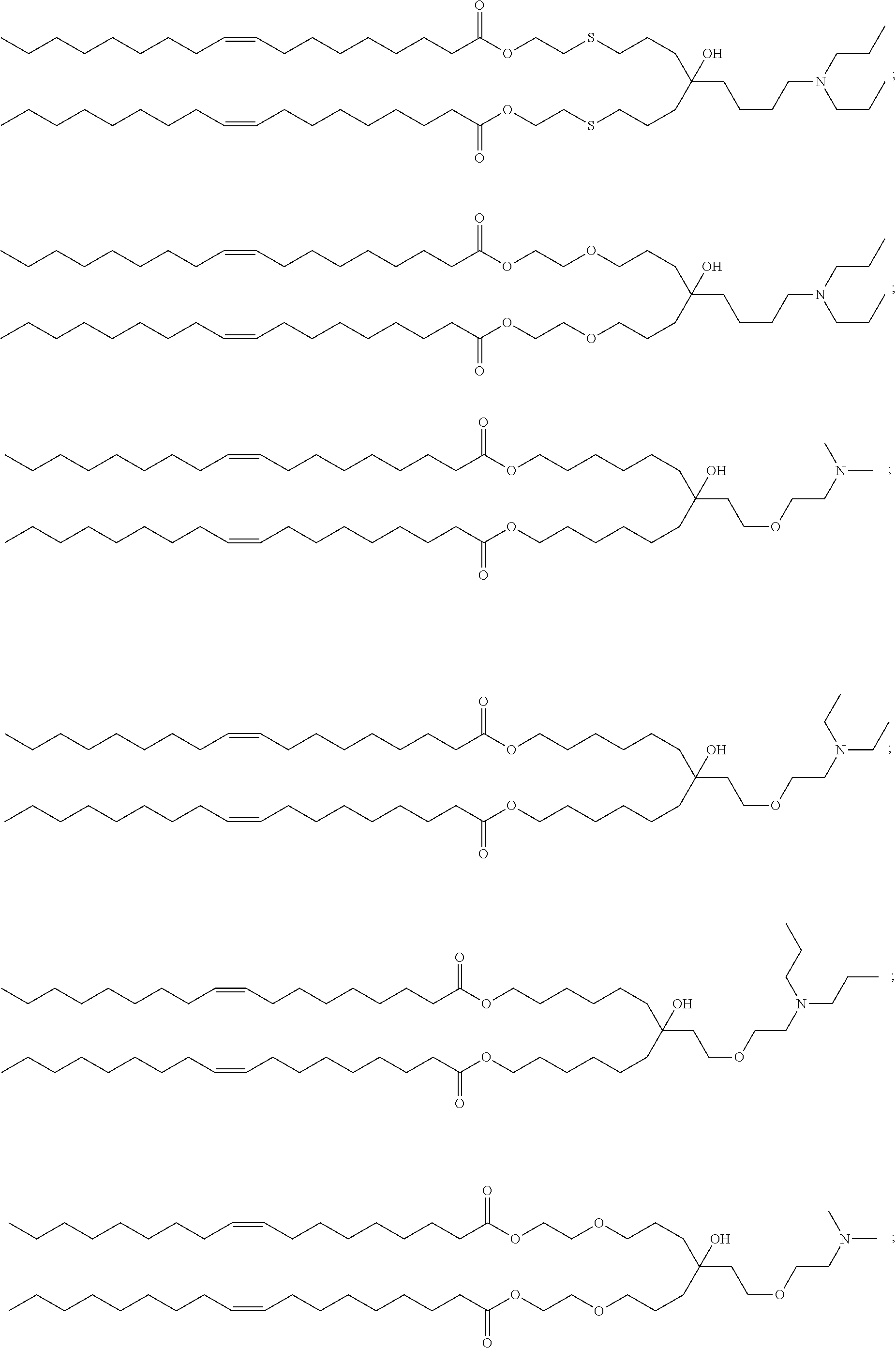

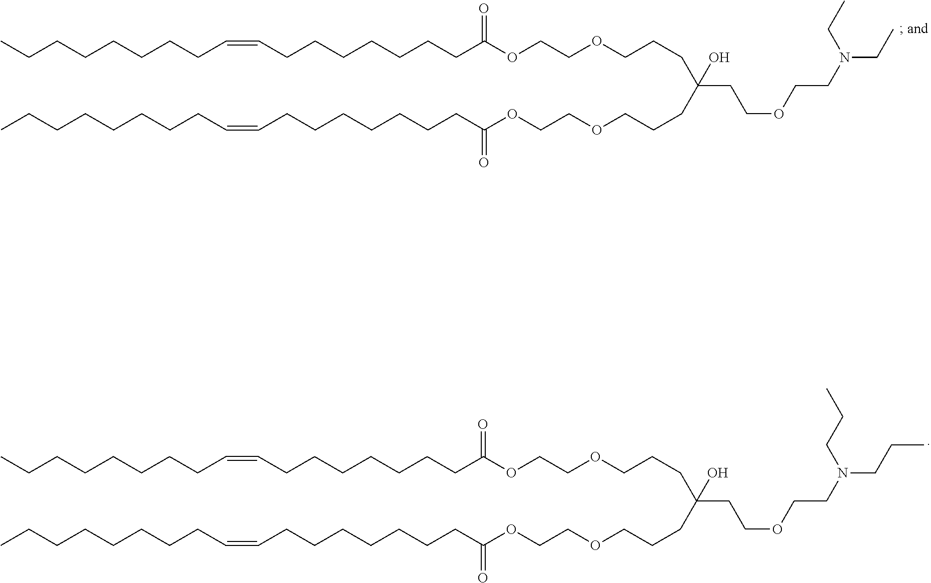

### Lipid Nanoparticles

The present disclosure provides for an LNP for delivery of a polynucleotide, such as an RNA, wherein the LNP comprises a compound of the present disclosure.

In embodiments, the LNPs have a mean diameter of from about 30 nm to about 160 nm, from about 40 nm to about 160 nm, from about 50 nm to about 160 nm, from about 60 nm to about 160 nm, from about 70 nm to about 160 nm, from about 50 nm to about 140 nm, from about 60 nm to about 130 nm, from about 70 nm to about 120 nm, from about 80 nm to about 120 nm, from about 90 nm to about 120 nm, from about 70 to about 110 nm, from about 80 nm to about 110 nm, or about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm or 160 nm. The diameter of the LNP may be measured by dynamic light scattering (DLS), transmission electron microscopy (TEM), scanning electron microscopy (SEM), or other methods such as are known in the art.

In some embodiments, the LNPs may be relatively homogenous. A polydispersity index may be used to indicate the homogeneity of the LNPs. A small, for example less than 0.3 or less than 0.2, polydispersity index generally indicates a narrow particle size distribution. A composition of the LNPs described herein may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of the LNP composition may be from about 0 to about 0.20 or 0.05 to 0.20.

The LNP may comprise more than one compound of Formula I or I-A to I-N or II-A to II-N, as appropriate. The inclusion of more than one such compound may, for example, be employed to achieve a desired pKa profile.

The LNP may comprise a compound of Formula I and an additional cationic and/or ionizable lipid, for example a cationic and/or ionizable lipid comprising a cyclic or non-cyclic amine. Such additional cationic and/or ionizable lipids may be selected from the non-limiting group consisting of:
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
(6Z,9Z,28Z,31Z)-heptatriacont-6,9,28,31-tetraene-19-yl 4-(dimethylamino)butanoate (DLin-MC3-DMA),
2,2-dilinoleyl-4-(2-dimethylaminoethyl)-[1,3]-dioxolane (DLin-KC2-DMA),
1,2-dioleyloxy-N,N-dimethylaminopropane (DODMA),
2-({8-[(3 p)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3 p)-cholest-5-en-3-yloxy]octyl}oxy)-N,N-dimethyl-3-[(9Z,12Z)-octadeca-9,12-die n-1-yloxy]propan-1-amine (Octyl-CLinDMA (2R)),
(2S)-2-({8-[(3p)-cholest-5-en-3-yloxy]octyl}oxy)-N,N-dimethyl-3-[(9Z,12Z)-octadeca-9,12-die n-1-yloxy]propan-1-amine (Octyl-CLinDMA (2S)),
((4-hydroxybutyl)azanediyl)bis(hexane-6,1-diyl)bis(2-hexyldecanoate)) and
8-[(2-hydroxyethyl)[6-oxo-6-(undecyloxy)hexyl]amino]-octanoic acid, 1-octylnonyl ester.

In embodiments, the LNP additionally comprises one or more of a PEG-lipid, a sterol structural lipid and/or a neutral lipid.

### PEGylated Lipids

In one embodiment, the present disclosure provides an LNP comprising a compound of the present disclosure and a PEGylated lipid.

It will be apparent to the skilled person that reference to a PEGylated lipid is a lipid that has been modified with polyethylene glycol. Exemplary PEGylated lipids include, but are not limited to, PEG-modified phosphatidylethanolamines, PEG-modified phosphatidic acids, PEG-modified ceramides, PEG-modified dialkylamines, PEG-modified diacylglycerols, and PEG-modified dialkylglycerols. For embodiment, a PEG lipid includes PEG-c-DOMG, PEG-DMG, PEG-DLPE, PEG-DMPE, PEG-DPPC, a PEG-DSPE lipid and combinations thereof.

### Neutral Lipids

In one embodiment, the present disclosure provides an LNP comprising a compound of the present disclosure and a neutral lipid.

Suitable neutral or zwitterionic lipids for use in the present disclosure will be apparent to the skilled person and include, in embodiments, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. The lipids can be saturated or unsaturated.

### Structural Lipids

In one embodiment, the present disclosure provides an LNP comprising a compound of the present disclosure and a structural lipid.

Exemplary structural lipids include, but are not limited to, cholesterol fecosterol, sitosterol, campesterol, stigmasterol, brassicasterol, ergosterol, tomatidine, tomatine, ursolic acid and alpha-tocopherol.

In one embodiment, the structural lipid is a sterol. In embodiments, the structural lipid is cholesterol. In another embodiment, the structural lipid is campesterol.

In embodiments, the LNPs comprise an ionizable cationic lipid compound of the present disclosure; a neutral lipid; a sterol such as cholesterol; and a PEGylated lipid. The LNPs are formulated with a polynucleotide to be delivered to a subject.

### Polynucleotides

The compounds of the present disclosure may form complexes with, and so be formulated into LNPs with, a range of polynucleotides including, but not limited to, mRNA, siRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), and the like. In this manner the LNPs and compositions may, in some embodiments, be used to induce expression of a desired protein both in vitro and in vivo by contacting cells with an LNP comprising one or more novel compounds of the present disclosure, wherein the LNP encapsulates or is associated with a polynucleotide that is expressed to produce the desired protein, such as a mRNA or plasmid encoding the desired protein. In alternative embodiments, the LNPs and compositions may be used to decrease the expression of target genes and proteins in vitro or in vivo by contacting cells with an LNP comprising one or more novel compounds of the present disclosure, wherein the LNP encapsulates or is associated with a polynucleotide that reduces target gene expression, such as an antisense oligonucleotide or siRNA.

Therefore, in some embodiments, the polynucleotide is a mRNA encoding a polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

In other embodiments, the polynucleotide is a siRNA capable of selectively knocking down or down regulating expression of a gene of interest. For example, an siRNA could be selected to silence a gene associated with a particular disease, disorder, or condition upon administration to a subject in need thereof of an LNP composition comprising the siRNA. A siRNA may comprise a sequence that is complementary to an mRNA sequence that encodes a gene or protein of interest. In some embodiments, the siRNA may be an immunomodulatory siRNA.

In some embodiments, the polynucleotide is a shRNA or a vector or plasmid encoding the same. A shRNA may be produced inside a target cell upon delivery of an appropriate construct to the nucleus. Constructs and mechanisms relating to shRNA are well-known in the relevant arts.

Polynucleotides useful for formulation with the LNPs incorporating an ionizable cationic compound of the present disclosure may include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, a polynucleotide further includes a poly-A region or a Kozak sequence (e.g., in the 5′-UTR). In some cases, polynucleotides may contain one or more intronic sequences capable of being excised from the polynucleotide. In some embodiments, a polynucleotide (e.g., an mRNA) may include a 5′ cap structure, a chain terminating nucleotide, a stem loop, a poly A sequence, and/or a polyadenylation signal. Any one of the regions of a polynucleotide may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxy uridine), a 1-substituted pseudouridine (e.g., 1-methyl-pseudouridine or 1-ethyl-pseudouridine), and/or a 5-substituted cytidine (e.g., 5-methyl-cytidine).

An exemplary polynucleotides useful for formulation with the LNPs incorporating an ionizable cationic compound of the present disclosure include a first region of linked nucleosides encoding an antigenic polypeptide, a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region.

Polynucleotides suitable for use with the present LNPs may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

In some embodiments, polynucleotides may include one or more alternative components, as described herein, which impart useful properties including increased stability and/or the lack of a substantial induction of the innate immune response of a cell into which the polynucleotide is introduced. For example, an alternative polynucleotide exhibits reduced degradation in a cell into which the polynucleotide is introduced, relative to a corresponding unaltered polynucleotide. These alternative species may enhance the efficiency of protein production, intracellular retention of the polynucleotides, and/or viability of contacted cells, as well as possess reduced immunogenicity.

Polynucleotides may be naturally or non-naturally occurring. Polynucleotides may include one or more modified (e.g., altered or alternative) nucleobases, nucleosides, nucleotides, or combinations thereof. The polynucleotides may include any useful modification or alteration, such as to the nucleobase, the sugar, or the internucleoside linkage (e.g., to a linking phosphate/to a phosphodiester linkage/to the phosphodiester backbone). In some embodiments, one or more alterations are present in each of the nucleobase, the sugar, and the internucleoside linkage.

Polynucleotides may or may not be uniformly altered along the entire length of the molecule. For example, one or more or all types of nucleotide (e.g., purine or pyrimidine, or any one or more or all of A, G, U, C) may or may not be uniformly altered in a polynucleotide, or in a given predetermined sequence region thereof.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus.

### Nucleobase Alternatives

The alternative nucleosides and nucleotides can include an alternative nucleobase. A nucleobase of a polynucleotide is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

Alternative nucleotide base pairing encompasses not only the standard adenine-thymine, adenine-uracil, or guanine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

In some embodiments, the nucleobase is an alternative uracil. Exemplary nucleobases and nucleosides having an alternative uracil include pseudouridine (y), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m3U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nm5s2U), 5-methylaminomethyl-uracil (mnm5U), 5-methylaminomethyl-2-thio-uracil (mnm5s2U), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnm5s2U), 5-propynyl-uracil, 1-propynyl-pseudouracil, 5-taurinomethyl-uracil (zm5U), 1-taurinomethyl-pseudouridine, 5-taurinomethyl-2-thio-uracil(zm5s2U), 1-taurinomethyl-4-thio-pseudouridine, 5-methyl-uracil (m5U, i.e., having the nucleobase deoxythymine), 1-methyl-pseudouridine (mΨ), 1-ethyl-pseudouridine (Et1Ψ), 5-methyl-2-thio-uracil (m5s2U), 1-methyl-4-thio-pseudouridine (mls4Ψ), 4-thio-1-methyl-pseudouridine, 3-methyl-pseudouridine (m3Ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp3U), 1-methyl-3-(3-amino-3-carboxypropyl)pseudouridine (acp3 y), 5-(isopentenylaminomethyl)uracil (inm5U), 5-(isopentenylaminomethyl)-2-thio-uracil (inm5s2U), 5,2′-O-dimethyl-uridine (m5Um), 2-thio-2′-O_methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil. In one example, the modified uracil is pseudouridine. In one example, the modified uracil is N1-methyl-pseudouridine.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-I-deaza-pseudoisocytidine, 1-methyl-I-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine. In one example, the modified cytosine is 5-methyl-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (mll), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m11m), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine.

Polynucleotides for formulation with LNPs comprising a compound of the present disclosure may be prepared according to any available technique known in the art. mRNA may be prepared by, for example, enzymatic synthesis which provides a process of template-directed synthesis of RNA molecules from an engineered DNA template comprised of an upstream bacteriophage promoter sequence linked to a downstream sequence encoding the gene of interest. Template DNA can be prepared for in vitro transcription from a number of sources with appropriate techniques which are well-known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012)

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well-known in the art. (see, e.g. Losick, R., 1972, In vitro transcription, Ann Rev Biochem v.41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five —In vitro transcription from plasmid or PCR-amplified DNA, Methods in Enzymology v. 530, 101-114; all of which are incorporated herein by reference).

The desired in vitro transcribed mRNA is then purified from the undesired components of the transcription or associated reactions. Techniques for the isolation of the mRNA transcripts are well known in the art and include phenol/chloroform extraction or precipitation with either alcohol in the presence of monovalent cations or lithium chloride.

### Lipid Nanoparticle Formation

LNPs comprising a compound of the present disclosure can be made using approaches which are well-known in the art of formulation. For example, suitable LNPs can be formed using mixing processes such as microfluidics, including herringbone micromixing, and T-junction mixing of two fluid streams, one of which contains the polynucleotide, typically in an aqueous solution, and the other of which has the various required lipid components, typically in ethanol.

The LNPs may then be prepared by combining a compound of Formula I, a phospholipid (such as DOPE or DSPC, which may be purchased from commercial sources including Avanti Polar Lipids, Alabaster, AL), a PEGylated lipid (such as 1,2-dimyristoyl-sn-glycerol methoxypoly ethylene glycol, also known as PEG-DMG, which may be purchased from commercial sources including Avanti Polar Lipids, Alabaster, AL), and a structural lipid/sterol (such as cholesterol, which may be purchased from commercial sources including Sigma-Aldrich), at concentrations of, for example, about 50 mM in ethanol. Solutions should be refrigerated during storage at, for example, −20 °C. The various lipids may be combined to yield the desired molar ratios and diluted with water and ethanol to a final desired lipid concentration of, for example, between about 5.5 mM and about 25 mM.

An LNP composition comprising a polynucleotide is prepared (as set out in the examples) by combining the above lipid solution with a solution including the polynucleotide at, for example, a lipid component to polynucleotide wt:wt ratio from about 5:1 to about 50:1. The lipid solution maybe rapidly injected using a NanoAssemblr microfluidic system at flow rates between about 3 ml/min and about 18 ml/min into the polynucleotide solution to produce a suspension with a water to ethanol ratio between about 1:1 and about 4:1, or between about 2:1 and about 4:1.

For LNP compositions including a RNA, solutions of the RNA at concentrations of 1.0 mg/ml in deionized water may be diluted in 50 mM sodium citrate buffer at a pH between 3 and 6 to form a stock solution.

LNP compositions may be further processed, as is known in the art, in one example by 10-fold dilution into 50 mM citrate buffer at pH 6 and subjected to tangential flow filtration (TFF) using a 300 k molecular weight cut-off membrane (mPES) until concentrated to the original volume. Subsequently, the citrate buffer may be replaced with a buffer containing 20 mM Tris buffer at pH 7.5, 80 mM sodium chloride, and 3% sucrose using diafiltration with a 10-fold volume of the new buffer. The LNP solution may be concentrated to a volume of between 5-10 mL, filtered using a 0.2 micron PES syringe filter, aliquoted into vials, and frozen at 1 °C/min using a Corning® CoolCell® LX Cell Freezing Container until the samples reach −80 °C. Samples may be stored at −80 °C until required.

The method described above induces nano-precipitation and particle formation. Alternative processes including, but not limited to, T-junction and direct injection, may be used to achieve the same nano-precipitation.

In some embodiments, the lipid component of the LNP formulation comprises about 25 mol % to about 60 mol % compound of Formula I, about 2 mol % to about 25 mol % phospholipid (neutral lipid), about 18.5 mol % to about 60 mol % structural lipid (sterol), and about 0.2 mol % to about 10 mol % of PEGylated lipid, provided that the total mol % does not exceed 100%. In some embodiments, the lipid component of the LNP formulation comprises about 30 mol % to about 50 mol % compound of Formula I, about 5 mol % to about 20 mol % phospholipid, about 30 mol % to about 55 mol % structural lipid, and about 1 mol % to about 5 mol % of PEGylated lipid. In a particular embodiment, the lipid component includes about 40 mol % compound of the present disclosure, about 10 mol % phospholipid, about 48 mol % structural lipid, and about 2.0 mol % of PEG lipid. In some embodiments, the phospholipid may be DOPE or DSPC. In other embodiments, the PEG lipid may be PEG-DMG and/or the structural lipid may be cholesterol.

The efficiency of encapsulation of the polynucleotide within the LNPs comprising a compound of the present disclosure may be at least 50%, for example about 50%, 55%, 60%, 65%, 70%, 75%, 80%, 85%, 90%, 91%, 92%, 93%, 94%, 95%, 96%, 97%, 98%, or 99%. In some embodiments, the encapsulation efficiency may be at least 80%. In certain embodiments, the encapsulation efficiency may be at least 90%.

### Lipid Nanoparticle Compositions

The LNPs comprising a compound of the present disclosure and a polynucleotide can be formulated for administration via any accepted mode of administration of lipid particles including LNPs, liposomes, lipid vesicles and like lipid-based particles. The pharmaceutical compositions of the invention may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical LNP compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. The compositions administered to a subject may be in the form of one or more dosage units, where for example, a tablet or injectable liquid volume may be a single dosage unit. Actual methods of preparing such dosage forms are known, or will be apparent, to those skilled in this art; for example, see Remington: The Science and Practice of Pharmacy, 20th Edition (Philadelphia College of Pharmacy and Science, 2000).

Therefore, one embodiment of the present disclosure provides a composition (such as a pharmaceutical composition) comprising an LNP, which comprises a compound of the present disclosure, combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for embodiment described in Remington's Pharmaceutical Sciences (Mack Publishing Co. N.J. USA, 1991).

An LNP is useful for parenteral, topical, oral, or local administration, intramuscular administration, aerosol administration, or transdermal administration, for prophylactic or for therapeutic treatment. In one embodiment, the LNP is administered parenterally, such as intramuscularly, subcutaneously or intravenously. In some embodiments, the LNP is administered intramuscularly.

Formulation of LNPs to be administered will vary according to the route of administration and formulation (e.g., solution, emulsion, capsule) selected. An appropriate pharmaceutical composition comprising an LNP to be administered can be prepared in a physiologically acceptable carrier. For solutions or emulsions, suitable carriers include, for embodiment, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for embodiment, sodium acetate, sodium chloride, potassium chloride, calcium chloride and sodium lactate. The LNPs can be stored in the liquid stage or can be lyophilized for storage and reconstituted in a suitable carrier prior to use according to art-known lyophilization and reconstitution techniques.

When the LNP composition is a vaccine composition then the carrier may be water, typically pyrogen-free water; isotonic saline or buffered (aqueous) solutions, e.g phosphate, citrate etc. buffered solutions. For injection of an LNP vaccine composition, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, such as at least 3 mM of a potassium salt. In an embodiment, the sodium, calcium and, optionally, potassium salts may be present as their chlorides, iodides, or bromides, or in the form of their hydroxides, carbonates, hydrogen carbonates, or sulfates, etc. Non-limiting examples of sodium salts include e.g. NaCl, NaI, NaBr, Na₂CO₃, NaHCO₃, Na₂SO₄, examples of the optional potassium salts include e.g. KCl, KI, KBr, K2CO₃, KHCO₃, K₂SO₄, and examples of calcium salts include e.g. CaCl₂, CaI₂, CaBr₂, CaCO₃, CaSO₄, Ca(OH)₂. Furthermore, organic anions of the aforementioned cations may be contained in the buffer. In certain embodiments, the buffer suitable for injection purposes, may contain salts selected from sodium chloride (NaCl), calcium chloride (CaCl₂)) and optionally potassium chloride (KCl), wherein further anions may be present additional to the chlorides. In embodiments, the salts in the injection buffer are present in a concentration of at least 50 mM sodium chloride (NaCl), at least 3 mM potassium chloride (KCl) and at least 0.01 mM calcium chloride (CaCl₂)). The injection buffer may be hypertonic, isotonic or hypotonic with reference to the specific reference medium.

In some embodiments of a vaccine, one or more compatible solid or liquid fillers or diluents or encapsulating compounds may be employed which are suitable for administration to a person. Pharmaceutically acceptable carriers, fillers and diluents will have sufficiently high purity and sufficiently low toxicity to make them suitable for administration to a subject. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; and alginic acid.

When the LNP composition is a vaccine composition it may further comprise one or more pharmaceutically acceptable adjuvants to enhance the immunostimulatory properties of the composition. The adjuvant may be any compound, which is suitable to support administration and delivery of the LNP composition and which may initiate or increase an immune response of the innate immune system, i.e. a non-specific immune response.

Such an adjuvant may be selected from any adjuvant known to a skilled person and suitable for the particular nature of the vaccine, i.e. for induction of a suitable immune response in a mammal. In embodiments, the adjuvant may be selected from the group consisting of MF59® (squalene-water emulsion), TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydroacetate); CALCITRIOL™ (1-alpha,25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(bl-4)-N-acetylmuramyl-L-alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-lbeta; interleukin-2; interleukin-7; interleukin-12; ISCOMS™; ISCOPREP 7.0.3.™; liposomes; LOXORIBINE™ (7-allyl-8-oxoguanosine); LT oral adjuvant (E. coli labile enterotoxin-protoxin); microspheres and microparticles of any composition; MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and D-MURAPALMITINE™ (Nac-Mur-L-Thr-D-isoGIn-sn-glyceroldipalmitoyl); NAGO (neuraminidase-galactose oxidase); nanospheres or nanoparticles of any composition; NISVs (non-ionic surfactant vesicles); PLEURAN™ (p-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, AL); STIMULON™ (QS—21); Quil-A (Quil-A saponin); S—28463 (4-amino-otec-dimethyl-2-ethoxymethyl-1H-imidazo[4,5 c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thrl]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin; microbe derived adjuvants, including Romurtide, DETOX, MPL, CWS, Mannose, CpG polynucleotide sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide. In one preferred example the adjuvant may be the oil-in-water emulsion adjuvant MF59®, particularly if the vaccine is an influenza vaccine.

Upon formulation, compositions of the present disclosure will be administered in a manner compatible with the dosage formulation and in such amount as is therapeutically/prophylactically effective. The dosage ranges for the administration of the LNPs are those large enough to produce the desired effect. For embodiment, the composition comprises an effective amount of the encapsulated or associated RNA, e.g., the mRNA or self-replicating RNA. In one embodiment, the composition comprises a therapeutically effective amount of the RNA. In another embodiment, the composition comprises a prophylactically effective amount of the RNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Preparation methods for the above compounds and compositions are described further herein and/or are known in the art.

### Methods of Treatment and Producing a Polypeptide of Interest

Diseases, disorders, and/or conditions which may be a result of or related to aberrant protein or polypeptide may be treated by the present LNPs comprising a compound of the present disclosure and a polynucleotide and may include, but are not limited to, rare diseases, infectious diseases, cancer and proliferative diseases, genetic diseases, autoimmune diseases, diabetes, neurodegenerative diseases, cardio- and reno-vascular diseases, and metabolic diseases.

LNP compositions may be formulated in unit dosage form. The therapeutically effective or prophylactically effective dose for any particular patient will depend upon a variety of factors including the severity and identify of a disorder being treated; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

LNP compositions described herein may be used in combination with one or more other therapeutic, prophylactic, diagnostic, or imaging agents. They may be administered together in a single composition or administered separately in different compositions.

The present disclosure provides methods of producing a polypeptide of interest in a mammalian cell. Methods of producing polypeptides involve contacting a cell with an LNP composition, as described herein, including an mRNA encoding the polypeptide of interest. Upon contacting the cell with the LNP composition, the mRNA may be taken up and translated in the cell to produce the polypeptide of interest.

The step of contacting an LNP composition including an mRNA with a cell may involve or cause transfection. A phospholipid including in the lipid component of the LNP composition may facilitate transfection and/or increase transfection efficiency, for example, by interacting and/or fusing with a cellular or intracellular membrane. Transfection may allow for the translation of the mRNA within the cell.

In some embodiments, the LNP compositions described herein may be used therapeutically. For example, an mRNA included in the LNP composition may encode a therapeutic polypeptide (e.g., in a translatable region) and produce the therapeutic polypeptide upon contacting and/or entry (e.g., transfection) into a cell. In other embodiments, an mRNA included in the LNP composition may encode a polypeptide that may improve or increase the immunity of a subject.

In embodiments, an mRNA included in an LNP composition may encode a recombinant polypeptide that may replace one or more polypeptides that may be substantially absent in a cell contacted with the LNP composition. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

In some embodiments, contacting a cell with an LNP composition including an mRNA may reduce the innate immune response of a cell to an exogenous polynucleotide. A cell may be contacted with a first LNP composition including a first amount of a first exogenous mRNA including a translatable region and the level of the innate immune response of the cell to the first exogenous mRNA may be determined. Subsequently, the cell may be contacted with a second LNP composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second LNP compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

In some embodiments, the present disclosure provides for the use of the LNPs comprising a compound of the present disclosure and a polynucleotide in the manufacture of a medicament for the treatment of a disease, disorder or condition. The disease, disorder or condition may be as described in any one or more embodiments herein.

The medicament may be for the prevention or treatment of a cancer, an infectious disease, an allergy, or an autoimmune disease. In embodiments, the medicament is a vaccine. The vaccine may be a tumor vaccine, an influenza vaccine, or a SARS-CoV-2 vaccine.

### Use of LNPs in a Vaccine

In some embodiments, the LNP comprising a compound of the present disclosure and a polynucleotide may be a component of a vaccine. Vaccines include compounds and preparations that are capable of providing immunity against one or more conditions related to infectious diseases and so may include mRNAs encoding infectious disease derived antigens and/or epitopes. Vaccines also include compounds and preparations that direct an immune response against cancer cells and can include mRNAs encoding tumor cell derived antigens, epitopes, and/or neoepitopes. Compounds eliciting immune responses may include vaccines, corticosteroids (e.g., dexamethasone), and other species.

In embodiments, the vaccine may be an mRNA vaccine and so the LNP comprising a compound of the present disclosure encapsulates or is associated with an mRNA molecule which comprises an mRNA sequence encoding an antigenic peptide or protein, or a fragment, variant or derivative thereof.

The antigenic peptides or proteins may be pathogenic antigens, tumour antigens, allergenic antigens or autoimmune self-antigens. Such pathogenic antigens may be those derived from pathogenic organisms, in particular bacterial, viral or protozoological (multicellular) pathogenic organisms, which evoke an immunological reaction in a mammalian subject, such as a human. Pathogenic antigens may be surface antigens, for example proteins or fragments thereof, located at the surface of the virus or the bacterial or protozoological organism.

Pathogenic antigens of interest may include those derived from one or more of: Acinetobacter baumannii, Anaplasma genus, Anaplasma phagocytophilum, Ancylostoma braziliense, Ancylostoma duodenale, Area nobacteri urn haemolyticum, Ascaris lumbricoides, Aspergillus genus, Astroviridae, Babesia genus, Bacillus anthracis, Bacillus cereus, Bartonella henselae, BK virus, Blastocysts hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, QD prion, Clonorchis sinensis, Clostridium botulinum, Clostridium difficile, Clostridium perfringens, Clostridium perfringens, Clostridium spp, Clostridium tetani, Coccidioides spp, coronavi ruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, 0111 and 0104: H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylon, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In certain embodiments, relevant antigens may be derived from the pathogens selected from: Severe Acute Respiratory Syndrome (SARS), Severe Acute Respiratory Syndrome Coronavirus and Severe Acute Respiratory Syndrome Coronavirus 2 (SARS-CoV-1 and SARS-CoV-2), Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, and Yellow Fever Virus.

In some embodiments, the relevant pathogenic antigen may be selected from: Outer membrane protein A OmpA, biofilm associated protein Bap, transport protein MucK (Acinetobacter baumannii, Acinetobacter infections)); variable surface glycoprotein VSG, microtubule-associated protein MAPP15, trans-sialidase TSA (Trypanosoma brucei, African sleeping sickness (African trypanosomiasis)); HIV p24 antigen, HIV envelope proteins (Gp120, Gp41, Gp160), polyprotein GAG, negative factor protein Nef, trans-activator of transcription Tat (HIV (Human immunodeficiency virus), AIDS (Acquired immunodeficiency syndrome)); galactose-inhibitable adherence protein GIAP, 29 kDa antigen Eh29, Gal/GalNAc lectin, protein CRT, 125 kDa immunodominant antigen, protein M17, adhesin ADH112, protein STIRP (Entamoeba histolytica, Amoebiasis); Major surface proteins 1-5 (MSP1a, MSP1b, MSP2, MSP3, MSP4, MSP5), type IV secreotion system proteins (VirB2, VirB7, VirB11, VirD4) (Anaplasma genus, Anaplasmosis); protective Antigen PA, edema factor EF, lethal facotor LF, the S-layer homology proteins SLH (Bacillus anthracis, Anthrax); acranolysin, phospholipase D, collagen-binding protein CbpA (Area nobacteri urn haemolyticum, Area nobacteri urn haemolyticum infection); nucleocapsid protein NP, glycoprotein precursor GPC, glycoprotein GPI, glycoprotein GP2 (Junin virus, Argentine hemorrhagic fever); chitin-protein layer proteins, 14 kDa suarface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp v13, allergen Asp f3, major conidial surface protein rodlet A, protease Peplp, GPI-anchored protein Gellp, GPI-anchored protein Crflp (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (a1, a2, b, c), 12D3, 11C5, 21B4, P29, variant erythrocyte surface antigen VESA1, Apical Membrane Antigen 1 AMA-1 (Babesia genus, Babesiosis); hemolysin, enterotoxin C, PXOl-51, glycolate oxidase, ABC-transporter, penicillin-binding protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocysts hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin binding protein A DbpA, decorin binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein vlsE (Borrelia genus, Borrelia infection); Botulinum neurotoxins BoNT/A1, BoNT/A2, BoNT/A3, BoNT/B, BoNT/C, BoNT/D, BoNT/E, BoNT/F, BoNT/G, recombinant botulinum toxin F He domain FHe (Clostridium botulinum, Botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein RpIL, OmpA-like transmembrane domain-containing protein Omp31, immunogenic 39-kDa protein M5 P39, zinc ABC transporter periplasmic zinc-bnding protein znuA, periplasmic immunogenic protein Bp26, 30S ribosomal protein S12 RpsL, glyceraldehyde-3-phosphate dehydrogenase Gap, 25 kDa outer-membrane immunogenic protein precursor Omp25, invasion protein B lalB, trigger factor Tig, molecular chaperone DnaK, putative peptidyl-prolyl cis-trans isomerase SurA, lipoprotein Ompl9, outer membrane protein MotY Ompl6, conserved outer membrane protein D15, malate dehydrogenase Mdh, component of the Type-IV secretion system (T4SS) VirJ, lipoprotein of unknown function BAB 1_0187 (Brucella genus, Brucellosis); members of the ABC transporter family (LolC, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, genome polyprotein, Sapoviurus capsid protein VP1, protein Vp3, geome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein PeblA, protein FspAl, protein FspA2 (Campylobacter genus, Campylobacteriosis); glycolytic enzyme enolase, secreted aspartyl proteinases SAPl-10, glycophosphatidylinositol (GPI)-linked cell wall protein, protein Hyrl, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LalB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Pan, mucin-Associated Surface Proteins MPSP (Trypanosoma cruzi, Chagas Disease (American trypanosomiasis)); envelope glycoproteins (gB, gC, gE, gH, gl, gK, gL), (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmpl, Pmp2, Pmp3, Pmp4, Pmp5, Pmp6, Pmp7, Pmp8, Pmp9, PmplO, Pmpll, Pmpl2, Pmpl3, Pmpl4, Pmpl5, Pmpl6, Pmpl7, Pmpl8, Pmpl9, Pmp20, Pmp21), (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis ptrotein F TcpF, cholera enterotoxin subunit A, cholera enterotoxin subunit B, Heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwpl3, cysteine protease Cwpl9, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: sprike proteins S, envelope proteins E, membrane proteins M, nucleocapsid proteins N (usually rhinoviruses and coronaviruses, Common cold (Acute viral rhinopharyngitis; Acute coryza)); prion protein Prp (CJD prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GalXM, glucuronoxylomannan GXM, mannoprotein MP (Cryptococcus neoformans, Cryptococcosis); acidic ribosomal protein P2 CpP2, mucin antigens Mud1, Muc2, Muc3 Muc4, Muc5, Muc6, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gpl5, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein ppl5, capsid-proximal tegument protein ppl50, protein M45, DNA polymerase UL54, helicase UL105, glycoprotein gM, glycoprotein gN, glcoprotein H, glycoprotein B gB, protein UL83, protein UL94, protein UL99 (Cytomegalovirus (CMV), Cytomegalovirus infection); capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoeba fragilis, Dientamoebiasis); diphtheria toxin precursor Tox, diphteria toxin DT, pilin-specific sortase SrtA, shaft pilin protein SpaA, tip pilin protein SpaC, minor pilin protein SpaB, surface-associated protein DIP1281 (Corynebacterium diphtheriae, Diphtheria); glycoprotein GP, nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vQD prion, Variant Creutzfeldt-Jakob disease (vCJD, nvCJD)); UvrABC system protein B, protein Flpl, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspAl, protein LspA2, protein LspB, outer membrane component DsrA, lectin DltA, lipoprotein Hip, major outer membrane protein OMP, outer membrane protein OmpA2 (Haemophilus ducreyi, Chancroid); aspartyl protease 1 Pepl, phospholipase B PLB, alpha-mannosidase 1 AMN1, glucanosyltransferase GEL1, urease URE, peroxisomal matrix protein Pmpl, proline-rich antigen Pra, humal T-cell reative protein TcrP (Coccidioides immitis and Coccidioides posadasii, Coccidioidomycosis); allergen Tri r 2, heat shock protein 60 Hsp60, fungal actin Act, antigen Tri r2, antigen Tri r4, antigen Tri tl, protein IV, glycerol-3-phosphate dehydrogenase Gpdl, osmosensor HwSholA, osmosensor HwSholB, histidine kinase HwHhk7B, allergen Mala s 1, allergen Mala s 11, thioredoxin Trx Mala s 13, allergen Mala f, allergen Mala s (usually Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins SalA and SalB, collagen adhesin Scm, surface proteins Fmsl (EbpA(fm), Fms5 (EbpB(fn), Fms9 (EpbC(fn) and FmslO, protein EbpC(fn), 96 kDa immunoprotective glycoprotein Gl (Enterococcus genus, Enterococcus infection); genome polyprotein, polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adrl (RP827), cell surface protein Adr2 (RP828), cell surface protein SCAl, Invasion protein invA, cell division protein fts, secretion proteins see Ofamily, virulence proteins virB, tlyA, tlyC, parvulin-like protein Pip, preprotein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein H, glycoprotein gp350, glycoprotein gpllO, glycoprotein gp42, glycoprotein gHgL, glycoprotein gB (Epstein-Barr Virus (EBV), Epstein-Barr Virus Infectious Mononucleosis); cpasid protein VP2, capsid protein VPi, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins LI and L2, Kunitz-type protein KTM, leucine aminopeptidase LAP, cysteine proteinase Fas2, saposin-like protein-2 SAP-2, thioredoxin peroxidases TPx, Prx-1, Prx-2, cathepsin I cysteine proteinase CL3, protease cathepsin L CLI, phosphoglycerate kinase PGK, 27-kDa secretory protein, 60 kDa protein HSP35alpha, glutathione transferase GST, 28.5 kDa tegumental antigen 28.5 kDa TA, cathepsin B3 protease CatB3, Type I cystatin stefin-1, cathepsin L5, cathepsin Llg and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, Iota toxin component 1b, protein CPE1281, pyruvate ferredoxin oxidoreductase, elongation factor G EF-G, perfringolysin 0 Pfo, glyceraldehyde-3-phosphate dehydrogenase GapC, Fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); phospholipase C PLC, heat-labile enterotoxin B, Iota toxin component 1b, protein CPE1281, pyruvate ferredoxin oxidoreductase, elongation factor G EF-G, perfringolysin 0 Pfo, glyceraldehyde-3-phosphate dehydrogenase GapC, fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (usually Clostridium perfringens; other Clostridium species, Gas gangrene (Clostridial myonecrosis)); lipase A, lipase B, peroxidase Decl (Geotrichum candidum, Geotrichosis); prion protein (GSS prion, Gerstmann-Straussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, alpha-giardin, alpha8-giardin, alphal-guiardin, beta-giardin, cystein proteases, glutathione-S-transferase GST, arginine deiminase ADI, fructose-1,6-bisphosphat aldolase FBA, Giardia trophozoite antigens GTA (GTA1, GTA2), ornithine carboxyl transferase OCT, striated fiber-asseblin-like protein SALP, uridine phosphoryl-like protein UPL, alpha-tubulin, beta-tubulin (Giardia intestinalis, Giardiasis); members of the ABC transporter family (LolC, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein (Burkholderia mallei, Glanders); cyclophilin CyP, 24 kDa third-stage larvae protien GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR (Neisseria gonorrhoeae, Gonorrhea); outer membrane protein A OmpA, outer membrane protein C OmpC, outer membrane protein K₁₇ OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emml, M protein type 6 Emm6, immunoglobulin-binding protein 35 Sib35, Surface protein R²⁸ Spr28, superoxide dismutase SOD, C₅a peptidase ScpA, antigen I/II Agl/II, adhesin AspA, G-related alpha2-macroglobulin-binding protein GRAB, surface fibrillar protein M5 (Streptococcus pyogenes, Group A streptococcal infection); C protein R antigen, arginine deiminase proteins, adhesin BibA, 105 kDA protein BPS, surface antigens c, surface antigens R, surface antigens X, trypsin-resistant protein RI, trypsin-resistant protein R³, trypsin-resistant protein R⁴, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alphalike protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein PI, outer membrane protein P2, outer membrane lipoprotein Pep, Lipoprotein E, outer membrane protein P4, fuculokinase FucK, [Cu, Zn]-superoxide dismutase SodC, protease HtrA, protein 0145, alpha-galactosylceramide (Haemophilus influenzae, Haemophilus influenzae infection); polymerase 3D, viral capsid protein VPl, viral capsid protein VP2, viral capsid protein VP3, viral capsid protein VP4, protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein S, envelope glycoprotein Gl, nucleoprotein NP, protein N, polyprotein M (Sin Nombre virus, Hantavirus, Hantavirus Pulmonary Syndrome (HPS)); heat shock protein HspA, heat shock protein HspB, citrate synthase GltA, protein UreB, heat shock protein Hsp60, neutrophil-activating protein NAP, catalase KatA, vacuolating cytotoxin VacA, urease alpha UreA, urease beta Ureb, protein CpnlO, protein groES, heat shock protein HsplO, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, 0-antigen, toxin-antigens Stx2B, toxin-antigen StxlB, adhesion-antigen fragment Int28, protein EspA, protein EspB, Intimin, protein Tir, protein IntC300, protein Eae (Escherichia coli O157:H7, 0111 and 0104:H4, Hemolytic-uremic syndrome (HUS)); RNA polymerase L, protein L, glycoprotein Gn, glycoprotein Gc, nucleocapsid protein S, envelope glycoprotein Gl, nucleoprotein NP, protein N, polyprotein M (Bunyaviridae family, Hemorrhagic fever with renal syndrome (HFRS)); glycoprotein G, matrix protein M, nucleoprotein N, fusion protein F, polymerase L, protein W, proteinC, phosphoprotein p, non-structural protein V (Henipavirus (Hendra virus Nipah virus), Henipavirus infections); polyprotein, glycoproten Gp2, hepatitis A surface antigen HBAg, protein 2A, virus protein VPl, virus protein VP2, virus protein VP3, virus protein VP4, protein PIB, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein, virus protein VPl, virus protein VP2, virus protein VP3, virus protein VP4 (Hepatitis B Virus (HBV), Hepatitis B); envelope glycoprotein E1 gp32 gp35, envelope glycoprotein E2 NSl gp68 gp70, capsid protein C, core protein Core, polyprotein, virus protein VPl, virus protein VP2, virus protein VP3, virus protein VP4, antigen G, protein NS3, protein NS5A, (Hepatitis C Virus, Hepatitis C); virus protein VPl, virus protein VP2, virus protein VP3, virus protein VP4, large hepaptitis delta antigen, small hepaptitis delta antigen (Hepatitis D Virus, Hepatitis D); virus protein VPl, virus protein VP2, virus protein VP3, virus protein VP4, capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase UL8, replication origin-binding protein UL9, glycoprotein M UL10, protein UL 11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein IE63 (ICP27, UL54), protein UL55, protein UL56, viral replication protein ICP22 (IE68, US1), protein US2, serine/threonine-protein kinase US3, glycoprotein G (US4), glycoprotein J (US5), glycoprotein D (US6), glycoprotein I (US7), glycoprotein E (US8), tegument protein US9, capsid/tegument protein US10, Vmw21 protein (US11), ICP47 protein (IE12, US12), major transcriptional activator ICP4 (IE175, RSI), E3 ubiquitin ligase ICPO (IE110), latency-related protein 1 LRP1, latency-related protein 2 LRP2, neurovirulence factor RL (ICP34.5), latency-associated transcript LAT (Herpes simplex virus 1 and 2 (HSV-1 and HSV-2), Herpes simplex); heat shock protein Hsp60, cell surface protein H1C, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, glutathione S-transferase GST, aspartic protease APR-1, acetylcholinesterase AChE (Ancylostoma duodenale and Necator americanus, Hookworm infection); protein NSI, protein NP1, protein VP1, protein VP2, protein VP3 (Human bocavirus (HBoV), Human bocavirus infection); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia ewingii, Human ewingii ehrlichiosis); major surface proteins 1-5 (MSP1a, MSP1b, MSP2, MSP3, MSP4, MSP5), type IV secreotion system proteins VirB2, VirB7, VirB11, VirD4 (Anaplasma phagocytophilum, Human granulocytic anaplasmosis (HGA)); protein NSI, small hydrophobic protein NS2, SH protein, fusion protein F, glycoprotein G, matrix protein M, matrix protein M2-1, matrix protein M2-2, phosphoprotein P, nucleoprotein N, polymerase L (Human metapneumovirus (hMPV), Human metapneumovirus infection); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia chaffeensis, Human monocytic ehrlichiosis); replication protein E1, regulatory protein E2, protein E3, protein E4, protein E5, protein E6, protein E7, protein E8, major capsid protein LI, minor capsid protein L2 (Human papillomavirus (HPV), Human papillomavirus (HPV) infection); fusion protein F, hemagglutinin-neuramidase HN, glycoprotein G, matrix protein M, phosphoprotein P, nucleoprotein N, polymerase L (Human parainfluenza viruses (HPIV), Human parainfluenza virus infection); Hemagglutinin (HA), Neuraminidase (NA), Nucleoprotein (NP), M1 protein, M2 protein, NSI protein, NS2 protein (NEP protein: nuclear export protein), PA protein, PB1 protein (polymerase basic 1 protein), PB1-F2 protein and PB2 protein (Orthomyxoviridae family, Influenza virus (flu)); genome polyprotein, protein E, protein M, capsid protein C (Japanese encephalitis virus, Japanese encephalitis); RTX toxin, type IV pili, major pilus subunit PilA, regulatory transcription factors PilS and PilR, protein sigma54, outer membrane proteins (Kingella kingae, Kingella kingae infection); prion protein (Kuru prion, Kuru); nucleoprotein N, polymerase L, matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase Fl, beta-tubulin, heat shock protein 70 Hsp70, KMP-11, glycoprotein GP63, protein BT1, nucleoside hydrolase NH, cell surface protein B1, ribosomal protein P1-like protein PI, sterol 24-c-methy transferase SMT, LACK protein, histone HI, SPB1 protein, thiol specific antioxidant TSA, protein antigen STIl, signal peptidase SP, histone H2B, suface antigen PSA-2, cystein proteinase b Cpb (Leishmania genus, Leishmaniasis); major membrane protein I, serine-rich antigen-45 kDa, 10 kDa caperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycPl or ML0041 coding protein, htrA2 or ML0176 coding protein, htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpLl, outer membrane protein LipL41, protein LigAni, surface protein LcpA, adhesion protein LipL53, outer membrane protein UpL32, surface protein Lsa63, flagellin FlaB1, membran lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin O precursor Hly (LLO), invasion-associated protein lap (P60), Listeriolysin regulatory protein PrfA, Zinc metalloproteinase Mpl, Phosphatidylinositol-specific phospholipase C PLC (PlcA, PlcB), 0-acetyltransferase Oat, ABC-transporter permease Im.G_1771, adhesion protein LAP, LAP receptor Hsp60, adhesin LapB, haemolysin listeriolysin OLLO, protein ActA, Internalin A InIA, protein InIB (Listeria monocytogenes, Listeriosis); outer surface protein A OspA, outer surface protein OspB, outer surface protein OspC, decorin binding protein A DbpA, decorin binding protein B DbpB, flagellar filament 41 kDa core protein Fla, basic membrane protein A BmpA (Immunodominant antigen P39), outer surface 22 kDa lipoprotein precursor (antigen IPLA7), variable surface lipoprotein vlsE (usually Borrelia burgdorferi and other Borrelia species, Lyme disease (Lyme borreliosis)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, protein Cox-2 (Wuchereria bancrofti and Brugia malayi, Lymphatic filariasis (Elephantiasis)); glycoprotein GP, matrix protein Z, polymerase L, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMA i, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP8, enolase PF10, hepatocyte erythrocyte protein 17 kDa HEP17, erythrocyte membrane protein 1 EMP1, protein Kbeta merozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, polymerase L, matrix protein VP40, envelope glycoprotein GP (Marburg virus, Marburg hemorrhagic fever (MHF)); protein C, matrix protein M, phosphoprotein P, non-structural protein V, hemagglutinin glycoprotein H, polymerase L, nucleoprotein N, fusion protein F (Measles virus, Measles); members of the ABC transporter family (LolC, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein, boaB coding protein (Burkholderia pseudomallei, Melioidosis (Whitmore's disease)); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR, factor H-binding protein fHbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar ot integral membrane protein YIPA, a nti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly(A) polymerase catalytic subunit PAPL, Major envelope protein P43K, early transcription factor 70 kDa subunit VETFS, early transcription factor 82 kDa subunit VETFL, metalloendopeptidase Gl-type, nucleoside triphosphatase I NPH1, replication protein A28-like MC134L, RNA polymease 7 kDa subunit RP07 (Molluscum contagiosum virus (MCV), Molluscum contagiosum (MC)); matrix protein M, phosphoprotein P/V, small hydrophobic protein SH, nucleoprotein N, protein V, fusion glycoprotein F, hemagglutinin-neuraminidase HN, RNA polymerase L (Mumps virus, Mumps); Outer membrane proteins OM, cell surface antigen OmpA, cell surface antigen OmpB (sca5), cell surface protein SCA4, cell surface protein SCA1, intracytoplasmic protein D, crystalline surface layer protein SLP, protective surface protein antigen SPA (Rickettsia typhi, Murine typhus (Endemic typhus)); adhesin PI, adhesion P30, protein p116, protein P40, cytoskeletal protein HMW1, cytoskeletal protein HMW2, cytoskeletal protein H/1W3, MPN152 coding protein, MPN426 coding protein, MPN456 coding protein, MPN-500coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Onchocerca volvulus, Onchocerciasis (River blindness)); 43 kDa secreted glycoprotein, glycoprotein gpO, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cystein protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, prtein PM0305, formate dehydrogenase-N, protein PM0698, protein PM1422, DNA gyrase, lipoprotein PlpE, adhesive protein Cp39, heme aquisition system receptor HasR, 39 kDa capsular protein, iron-regulated OMP IROMP, outer membrane protein OmpA87, fimbrial protein Ptf, fimbrial subunit protein PtfA, transferrin binding protein Tbpl, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Pm, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Pm, protein Fim2, protein Fim3; “(Bordetella pertussis, Pertussis (Whooping cough)); “Fl capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YP00612, heat shock protein 90 HtpG, putative sulfatase protein YdeN, outer-membrane lipoprotein carrier protein LolA, secretion chaperone YerA, putative lipoprotein YP00420, hemolysin activator protein HpmB, pesticin/yersiniabactin outer membrane receptor Psn, secreted effector protein YopE, secreted effector protein YopF, secreted effector protein YopK, outer membrane protein YopN, outer membrane protein YopM, Coagulase/fibrinolysin precursor Pla; “(Yersinia pestis, Plague); protein PhpA, surface adhesin PsaA, pneumolysin Ply, ATP-dependent protease CIp, lipoate-protein ligase LplA, cell wall surface anchored protein psrP, sortase SrtA, glutamyl-tRNA synthetase GltX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A12, 55 kDa antigen P55, major surface glycoprotein Msg (Pneumocystis jirovecii, Pneumocystis pneumonia (PCP)); genome polyprotein, polymerase 3D, viral capsid protein VP1, viral capsid protein VP2, viral capsid protein VP3, viral capsid protein VP4, protease 2A, protease 3C (Poliovirus, Poliomyelitis); protein Nfal, exendin-3, secretory lipase, cathepsin B-like protease, cysteine protease, cathepsin, peroxiredoxin, protein CrylAc (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmpl, Pmp2, Pmp3, Pmp4, Pmp5, Pmp6, Pmp7, Pmp8, Pmp9, PmplO, Pmpll, Pmpl2, Pmpl3, Pmpl4, Pmpl5, Pmpl6, Pmpl7, Pmpl8, Pmpl9, Pmp20, Pmp21) (Chlamydophila psittaci, Psittacosis); outer membrane protein PI, heat shock protein B HspB, peptide ABC transporter, GTP-binding protein, protein IcmB, ribonuclease R, phosphatas SixA, protein DsbD, outer membrane protein TolC, DNA-binding protein PhoB, ATPase DotB, heat shock protein B HspB, membrane protein Coml, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L, phophoprotein P, matrix protein M, glycoprotein G (Rabies virus, Rabies); fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 (Respiratory syncytial virus (RSV), Respiratory syncytial virus infection); genome polyprotein, polymerase 3D, viral capsid protein VP1, viral capsid protein VP2, viral capsid protein VP3, viral capsid protein VP4, protease 2A, protease 3C (Rhinovirus, Rhinovirus infection); outer membrane proteins OM, cell surface antigen OmpA, cell surface antigen OmpB (sca5), cell surface protein SCA4, cell surface protein SCAl, protein PS120, intracytoplasmic protein D, protective surface protein antigen SPA (Rickettsia genus, Rickettsial infection); outer membrane proteins OM, cell surface antigen OmpA, cell surface antigen OmpB (sca5), cell surface protein SCA4, cell surface protein SCAl, intracytoplasmic protein D (Rickettsia akari, Rickettsialpox); envelope glycoprotein GP, polymerase L, nucleoprotein N, non-structural protein NSS (Rift Valley fever virus, Rift Valley fever (RVF)); outer membrane proteins OM, cell surface antigen OmpA, cell surface antigen OmpB (sca5), cell surface protein SCA4, cell surface protein SCAl, intracytoplasmic protein D (Rickettsia rickettsii, Rocky mountain spotted fever (RMSF)); non-structural protein 6 NS6, non-structural protein 2 NS2, intermediate capsid protein VP6, inner capsid protein VP2, non-structural protein 3 NS3, RNA-directed RNA polymerase L, protein VP3, non-structural protein 1 NS1, non-structural protein 5 NS5, outer capsid glycoprotein VP7, nonstructural glycoprotein 4 NS4, outer capsid protein VP4; (Rotavirus, Rotavirus infection); polyprotein P200, glycoprotein E1, glycoprotein E2, protein NS2, capsid protein C (Rubella virus, Rubella); chaperonin GroEL (MopA), inositol phosphate phosphatase SopB, heat shock protein HsIU, chaperone protein DnaJ, protein TviB, protein IroN, flagellin FliC, invasion protein SipC, glycoprotein gp43, outer membrane protein LamB, outer membrane protein PagC, outer membrane protein TolC, outer membrane protein NmpC, outer membrane protein FadL, transport protein SadA, transferase WgaP, effector proteins SifA, SteC, SseL, SseJ and SseF (Salmonella genus, Salmonellosis); “protein 14, non-structural protein NS7b, non-structural protein NS8a, protein 9b, protein 3a, nucleoprotein N, non-structural protein NS3b, non-structural protein NS6, protein 7a, non-structural protein NS8b, membrane protein M, envelope small membrane protein EsM, replicase polyprotein 1a, spike glycoprotein S, replicase polyprotein lab; SARS coronavirus, SARS (Severe Acute Respiratory Syndrome)); serin protease, Atypical Sarcoptes Antigen 1 ASA1, glutathione S-transferases GST, cystein protease, serine protease, apolipoprotein (Sarcoptes scabiei, Scabies); glutathione S-transferases GST, paramyosin, hemoglbinase SM32, major egg antigen, 14 kDa fatty acid-binding protein Sm14, major larval surface antigen P37, 22.6 kDa tegumental antigen, calpain CANP, triphospate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator IE63, transcriptional transactivator IE62, protease P33, alpha trans-inducing factor 74 kDa protein, deoxyuridine 5′-triphosphate nucleotidohydrolase, transcriptional transactivator IE4, membrane protein UL43 homolog, nuclear phosphoprotein UL3 homolog, nuclear protein UL4 homolog, replication origin-binding protein, membrane protein 2, phosphoprotein 32, protein 57, DNA polymerase processivity factor, portal protein 54, DNA primase, tegument protein UL14 homolog, tegument protein UL21 homolog, tegument protein UL55 homolog, tripartite terminase subunit UL33 homolog, tri partite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein CI, protein C5, protein C6, protein F7, protein F8, protein F9, protein Fl1, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntCl, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntCl, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus e.g. aureus, Staphylococcal infection); antigen Ss-IR, antigen NIE, strongylastacin, Na+-K+ATPase Sseat-6, tropomysin SsTmy-1, protein LEC-5, 41 kDa antigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpF1, repeat protein Tpr, repeat protein F TprF, repeat protein G TprG, repeat protein I Tprl, repeat protein J TprJ, repeat protein K TprK, treponemal membrane protein A TmpA, lipoprotein, 15 kDa Tppl5, 47 kDa membrane antigen, miniferritin TpF1, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); genome polyprotein, protein E, protein M, capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES—70, glycoprotein GP31, excretory-secretory antigen TcES—57, perienteric fluid antigen Pe, soluble extract antigens Ex, excretory/secretory larval antigens ES, antigen TES—120, polyprotein allergen TBA-1, cathepsin L-like cysteine protease c-cpl-1, 26-kDa protein (Toxocara canis or Toxocara cati, Toxocariasis (Ocular Larva Migrans (OLM) and Visceral Larva Migrans (VLM))); microneme proteins (MICl, MIC2, MIC3, MIC4, MIC5, MIC6, MIC7, MIC8), rhoptry protein Rop2, rhoptry proteins (Ropl, Rop2, Rop3, Rop4, Rop5, Rop6, Rop7, Ropl6, Rjopl7), protein SRl, surface antigen P22, major antigen p24, major surface antigen p30, dense granule proteins (GRA1, GRA2, GRA3, GRA4, GRA5, GRA6, GRA7, GRA8, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Mybl, Myb2, Myb3), adhesion protein AP23, adhesion protein AP33, adhesin protein AP33-3, adhesins AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Enol, cysteine proteinase CP30, heat shock proteins (Hsp70, Hsp60), immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone DnaK, cell surface lipoprotein Mpt83, lipoprotein P23, phosphate transport system permease protein pstA, 14 kDa antigen, fibronectin-binding protein C FbpCl, Alanine dehydrogenase TB43, Glutamine synthetase 1, ESX-1 protein, protein CFP10, TB10.4 protein, protein MPT83, protein MTB12, protein MTB8, Rpf-like proteins, protein MTB32, protein MTB39, crystallin, heat-shock protein HSP65, protein PST-S (usually Mycobacterium tuberculosis, Tuberculosis); outer membrane protein FobA, outer membrane protein FobB, intracellular growth locus IglCl, intracellular growth locus IglC2, aminotransferase Wbtl, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein tolC, FAD binding family protein, type IV pilin multimeric outer membrane protein, two component sensor protein KdpD, chaperone protein DnaK, protein TolQ (Francisella tularensis, Tularemia); “MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen; (Ureaplasma urealyticum, Ureaplasma urealyticum infection); non-structural polyprotein, structural polyprotein, capsid protein CP, protein E1, protein E2, protein E3, protease PI, protease P2, protease P3 (Venezuelan equine encephalitis virus, Venezuelan equine encephalitis); glycoprotein GP, matrix protein Z, polymerase L, nucleoprotein N (Guanarito virus, Venezuelan hemorrhagic fever); polyprotein, protein E, protein M, capsid protein C, protease NS3, protein NS1, protein NS2A, protein AS2B, brotein NS4A, protein NS4B, protein NS5 (West Nile virus, West Nile Fever); cpasid protein CP, protein E1, protein E2, protein E3, protease P2 (Western equine encephalitis virus, Western equine encephalitis); genome polyprotein, protein E, protein M, capsid protein C, protease NS3, protein NS1, protein NS2A, protein AS2B, protein NS4A, protein NS4B, protein NS5 (Yellow fever virus, Yellow fever); putative Yop targeting protein YobB, effector protein YopD, effector protein YopE, protein YopH, effector protein YopJ, protein translocation protein YopK, effector protein YopT, protein YpkA, flagellar biosyntheses protein FlhA, peptidase M48, potassium efflux system KefA, transcriptional regulatoer RovA, adhesin Ifp, translocator portein LcrV, protein PcrV, invasin Inv, outer membrane protein OmpF-like porin, adhesin YadA, protein kinase C, phospholipase CI, protein PsaA, mannosyltransferase-like protein WbyK, protein YscU, antigen YPMa (Yersinia pseudotuberculosis, Yersinia pseudotuberculosis infection); effector protein YopB, 60 kDa chaperonin, protein WbcP, tyrosin-protein phosphatase YopH, protein YopQ, enterotoxin, Galactoside permease, reductaase NrdE, protein YasN, Invasin Inv, adhesin YadA, outer membrane porin F OmpF, protein UspAl, protein EibA, protein Hia, cell surface protein Ail, chaperone SycD, protein LcrD, protein LcrG, protein LcrV, protein SycE, protein YopE, regulator protein TyeA, protein YopM, protein YopN, protein YopO, protein YopT, protein YopD, protease ClpP, protein MyfA, protein FilA, and protein PsaA (Yersinia enterocolitica, Yersiniosis).

In embodiments wherein the infectious disease is influenza, the mRNA molecule may have a coding region encoding at least one antigenic peptide or protein derived from hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, or polymerase basic protein 2 (PB2) of an influenza virus or a fragment or variant thereof.

In certain embodiments, the coding region encodes at least one antigenic peptide or protein derived from hemagglutinin (HA) and/or neuraminidase (NA) of an influenza virus or a fragment or variant thereof. The HA and/or NA may, independently, be derived from an influenza A virus or an influenza B virus or a fragment of either.

In embodiments wherein the infectious disease is influenza, the mRNA molecule may have a coding region encoding at least one antigenic peptide or protein derived from Spike (S) protein.

### EXAMPLES

### Example 1: Synthetic Approach to Compounds SL26 to SL33

The following synthetic procedures for compounds SL26 to SL33 describe approaches by which compounds of the present disclosure may be made. These synthetic approaches would be known to a person of skill in the art. Simple modifications of conditions or the nature of any particular substrate may be changed to achieve any compound within the scope of Formula I.

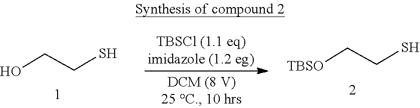

To a solution of compound 1 (99.5 g, 1.27 mol, 88.8 mL, 1.00 eq) and imidazole (104 g, 1.53 mol, 1.20 eq) in DCM (800 mL) was added TBSCl (211 g, 1.40 mol, 171 mL, 1.10 eq) in portions at 25 °C. After addition, the resulting suspension was stirred at 25 °C for 10 hrs. TLC (petroleum ether/ethyl acetate=5/1, Rf of compound 1=0.74, Rf of compound 2=0.43) showed the reaction was finished. The reaction solution was poured into water (300 mL) and extracted with EtOAc (100 mL). The combined organic layer was washed with brine (200 mL), dried over sodium sulfate and concentrated to give product. The residue was purified by column chromatography (SiO₂, petroleum ether/ethyl acetate=100/1 to 0/1) to give compound 2 (100 g, 519 mmol, 40.8% yield) as colorless oil. ¹H NMR: ET61249-5-P1A (400 MHz, CDCl₃) δ 3.73 (t, J=6.4 Hz, 2H), 2.60-2.65 (m, 2H), 1.53 (t, J=8.4 Hz, 2H), 0.87 (s, 9H), 0.05 (s, 6H).

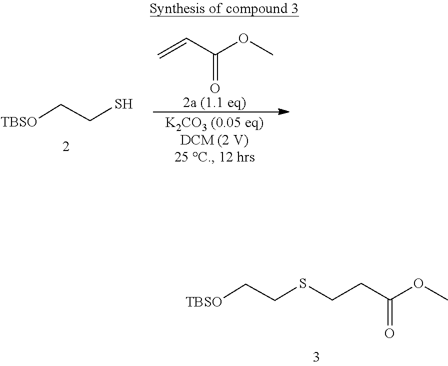

To a solution of compound 2 (35.0 g, 181 mmol, 1.00 eq) in DCM (70.0 mL) was added K₂CO₃ (1.26 g, 9.10 mmol, 0.05 eq). Compound 2a (17.2 g, 200 mmol, 18.0 mL, 1.10 eq) was added dropwise to the solution. The solution was stirred at 25 °C for 12 hrs. TLC (petroleum ether/ethyl acetate=3/1, Rf of compound 2=0.7, Rf of compound 3=0.38) showed the reaction solution was finished. The reaction was poured into water (300 mL). The solution was extracted with EtOAc (100 mL×2) and the organic layers were combined and concentrated in vacuum to give a residue. The residue was purified by column chromatography (SiO₂, petroleum ether/ethyl acetate=100/1 to 0/1) to give compound 3 (48.0 g, crude) as colorless oil. ¹H NMR: ET61249-6-P1A (400 MHz, CDCl₃) δ 5.29 (s, 1H), 3.68-3.78 (m, 2H), 3.68 (s, 3H), 2.67-2.84 (m, 2H), 2.59-2.65 (m, 4H), 0.87 (s, 9H), 0.05 (s, 6H).

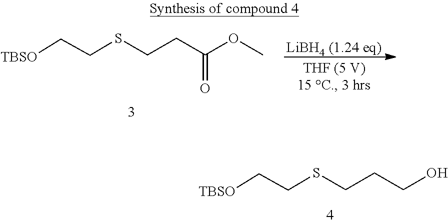 [SMILES: COC(=O)CCSCCO[Si](C)(C)C(C)(C)C ; CC(C)(C)[Si](C)(C)OCCSCCCO]

To a solution of compound 3 (35.0 g, 125 mmol, 1.00 eq) in THF (175 mL) under N₂ was added LiBH₄ (3.40 g, 156 mmol, 1.24 eq) in portions at 0 °C. The solution was stirred at 15 °C for 3 hrs. TLC (petroleum ether/ethyl acetate=5/1, Rf of compound 3=0.73, Rf of compound 4=0.42) showed that the reaction was finished. The solution was poured into NH₄Cl (aq. sat. 200 mL). The solution was extracted with EtOAc (50.0 mL×2). The organic layers were combined and concentrated in vacuum to give compound 4 (28.0 g, 112 mmol, 88.9% yield) as colorless oil. ¹H NMR: ET61249-12-P1A (400 MHz, CDCl₃) δ 3.74-3.79 (m, 4H), 2.64-2.70 (m,4H), 1.82-1.88 (m, 2H), 0.90 (s, 9H), 0.08 (s, 6H).

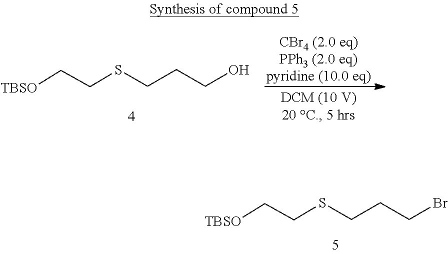 [SMILES: CC(C)(C)[Si](C)(C)OCCSCCCO ; CC(C)(C)[Si](C)(C)OCCSCCCBr]

To a solution of compound 4 (40.0 g, 160 mmol, 1.00 eq) in DCM (400 mL) was added CBr₄ (106 g, 319 mmol, 2.00 eq), pyridine (126 g, 1.60 mol, 129 mL, 10.0 eq) and PPh₃ (83.8 g, 319 mmol, 2.00 eq). The mixture was stirred at 20 °C for 5 hrs. TLC (petroleum ether/ethyl acetate=10/1, Rf of compound 4=0.23, Rf of compound 5=0.65) showed the reaction was finished. The reaction solution was concentrated under reduced pressure to give a residue. The residue was purified by column chromatography (SiO₂, petroleum ether/ethyl acetate=1/0). Compound 5 (25.0 g, 79.8 mmol, 50.0% yield) was obtained as a colorless oil. ¹H NMR: ET60350-30-P1A1 (400 MHz, CDCl₃) δ 0.04-0.10 (m, 6H), 0.86-0.92 (m, 9H), 2.06-2.15 (m, 2H), 2.61-2.73 (m, 4H), 3.47-3.53 (m, 2H), 3.73-3.79 (m, 2H).

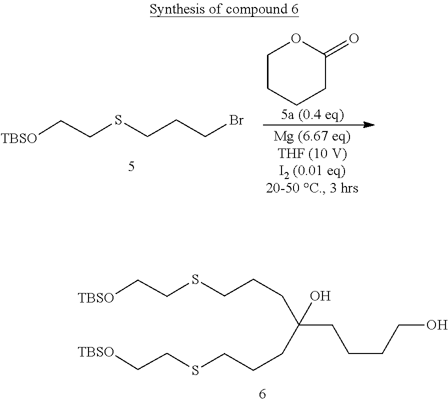

To a suspension of Mg (10.3 g, 426 mmol, 6.67 eq) and 12 (1.62 g, 6.38 mmol, 1.29 mL, 0.10 eq) in THF (40.0 mL) was dropwise added compound 5 (20.0 g, 63.8 mmol, 1.00 eq) at 50 °C over 30 min. After the addition, the solution was stirred at 50 °C for 30 min. Then compound 5a (2.56 g, 25.5 mmol, 2.30 mL, 0.4 eq) was added dropwise at 50 °C. The resulting solution was stirred at 25 °C for 2 hrs. TLC (petroleum ether/ethyl acetate=1/1, Rf of compound 5=0.76, Rf of compound 6=0.32) showed that the reaction solution was finished. The residue was purified by column chromatography (SiO₂, petroleum ether/ethyl acetate=50/1 to 0/1) to give compound 6 (14.0 g, 24.6 mmol, 77.1% yield) as colorless oil. ¹H NMR: ET61249-20-P1A (400 MHz, CDCl₃) δ 3.76 (t, J=6.8 Hz, 4H), 3.67 (s, 2H), 2.64 (t, J=7.2 Hz, 4H), 2.55-2.26 (m, 4H), 1.52-1.57 (m, 10H), 0.90 (s, 18H), 0.08 (s, 12H).

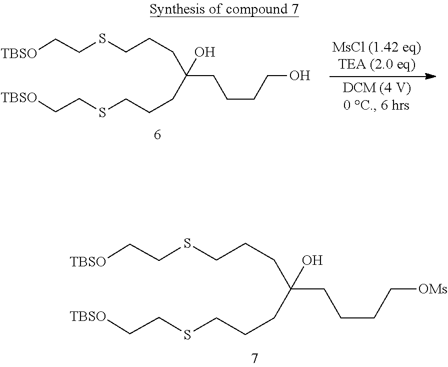 [SMILES: CC(C)(C)[Si](C)(C)OCCSCCCC(O)(CCCCO)CCCSCCO[Si](C)(C)C(C)(C)C ; CC(C)(C)[Si](C)(C)OCCSCCCC(O)(CCCCOS(C)(=O)=O)CCCSCCO[Si](C)(C)C(C)(C)C]

To a solution of compound 6 (3.50 g, 6.15 mmol, 1.00 eq) and TEA (1.24 g, 12.3 mmol, 1.71 mL, 2.00 eq) in DCM (15.0 mL) was added dropwise MsCl (1.00 g, 8.73 mmol, 676 μL, 1.42 eq) at 0 °C. After addition, the solution was stirred at 0 °C for 6 hrs. TLC (petroleum ether/ethyl acetate=1/1, Rf of compound 6=0.24, Rf of compound 7=0.37) showed that the reaction solution was finished. The solution was poured into NH₄Cl (aq. sat. 50.0 mL). The solution was extracted with EtOAc (20.0 mL×2). The organic layers were combined and concentrated in vacuum to give compound 7 (3.90 g, 6.03 mmol, 97.9% yield) as yellow oil.

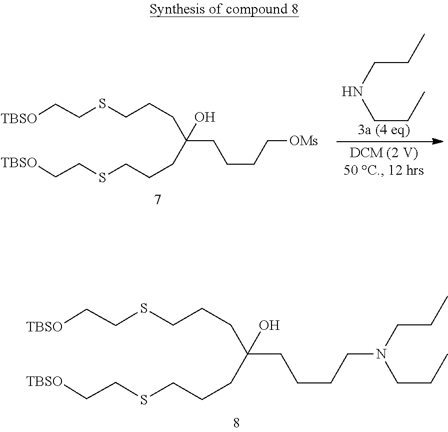

To a solution of compound 3a (2.19 g, 21.6 mmol, 2.98 mL, 4.00 eq) in DCM (8.00 mL) was added compound 7 (3.50 g, 5.41 mmol, 1.00 eq). The solution was stirred at 50 °C for 12 hrs. TLC (DCM/MeOH=8/1, Rf of compound 7=0.65, Rf of compound 8=0.40) showed the reaction was finished. The organic layers were combined and concentrated in vacuum to give crude product. The residue was purified by column chromatography (SiO₂, petroleum ether/ethyl acetate=10/1 to 0/1) to give compound 8 (3.00 g, 4.60 mmol, 85.1% yield) as yellow oil.

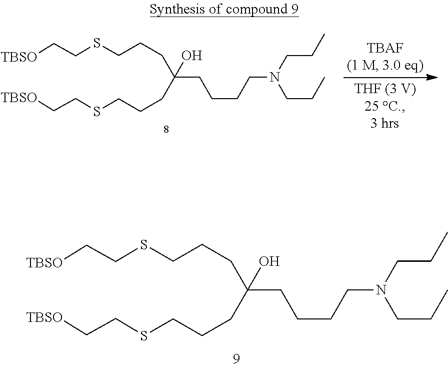 [SMILES: CCCN(CCC)CCCCC(O)(CCCSCCO[Si](C)(C)C(C)(C)C)CCCSCCO[Si](C)(C)C(C)(C)C ; CCCN(CCC)CCCCC(O)(CCCSCCO[Si](C)(C)C(C)(C)C)CCCSCCO[Si](C)(C)C(C)(C)C]

To a solution of compound 8 (3.00 g, 4.60 mmol, 1.00 eq) in THF (10.5 mL) was added TBAF (1.00 M, 13.8 mL, 3.00 eq). The solution was stirred at 25 °C for 3 hrs. TLC (DCM/MeOH=8/1, Rf of compound 8=0.52, Rf of compound 9=0.26) showed that the reaction was finished. The reaction solution was concentrated under vacuum to give compound 9 (6.00 g, crude) as yellow oil.

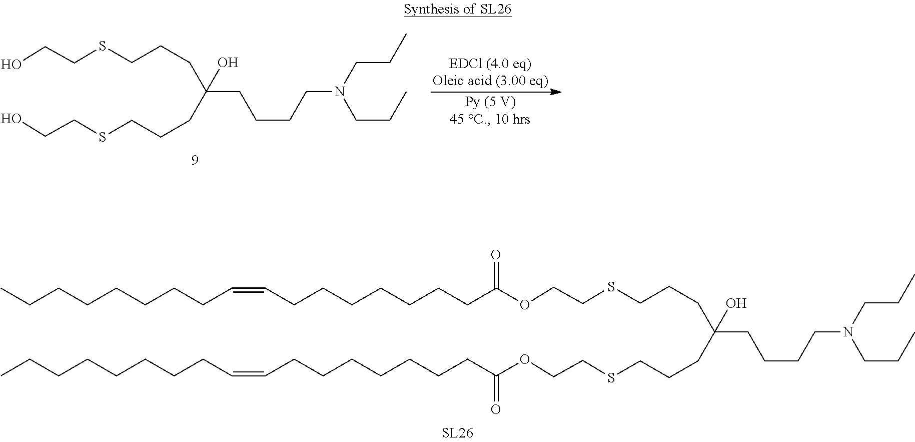 [SMILES: CCCN(CCC)CCCCC(O)(CCCSCCO)CCCSCCO ; CCCCCCCC/C=C\CCCCCCCC(=O)OCCSCCCC(O)(CCCCN(CCC)CCC)CCCSCCOC(=O)CCCCCCC/C=C\CCCCCCCC]

To a solution of compound 9 (2.00 g, 4.72 mmol, 1.00 eq) in Py (10.0 mL) was added Oleic acid (4.00 g, 14.2 mmol, 4.00 mL, 3.00 eq) and EDCI (3.62 g, 18.9 mmol, 4.00 eq). The mixture was stirred at 45 °C for 10 hrs. LCMS showed compound 9 was consumed completely. Several new peaks were shown on LCMS and desired compound was detected. The reaction mixture was partitioned between EtOAc (200 mL) and water (100 mL). The organic layer was dried over Na₂SO₄ and concentrated under reduced pressure to give the crude product. The crude product was purified by prep-HPLC (column: Welch Xtimate C1 100*30 mm*5 um; mobile phase: [Water (0.225% FA)-(ACN-THF 2/1)]; B %: 40%-85%, 20 min) to give SL26 (1.00 g, 1.05 mmol, 22.3% yield) as yellow oil. LCMS: ET60350-32-P1A (RT=2.916 min, M+H⁺=952.9). ¹H NMR: ET60350-32-P1A (400 MHz, CDCl₃) δ 5.31-5.39 (m, 4H), 4.19-4.25 (m, 4H), 2.72-2.78 (m, 4H), 2.54-2.61 (m, 4H), 2.28-2.47 (m, 10H), 1.98-2.05 (m, 8H), 1.61-1.68 (m, 10H), 1.43-1.55 (m, 12H), 1.26-1.34 (m, 40H), 0.85-0.91 (m, 12H).

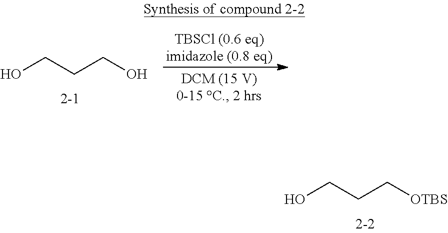 [SMILES: "TBSCl (0.6 eq)imidazole (0.8 eq)" ; OCCCO ; CC(C)(C)[Si](C)(C)OCCCO]

To a solution of compound 2-1 (50.0 g, 657 mmol, 47.6 mL, 1.00 eq) in DCM (750 mL) was added imidazole (35.7 g, 525 mmol, 0.80 eq) at 15 °C. Then TBSCl (59.4 g, 394 mmol, 48.3 mL, 0.60 eq) was added into the mixed solution at 0 °C in portions maintaining internal temperature below 10 °C. The resulting solution was stirred at 15 °C for 2 hrs. TLC (petroleum ether/ethyl acetate=1/1, Rf of compound 2-1=0.10, and Rf of compound 2-2=0.50) indicated none of TBSCl remained, and two new major spots with lower polarity were detected. The reaction solution was poured into the saturated aqueous solution of NH₄Cl (500 ml), and the organic phase was washed by half-saturated aqueous solution of NaCl (200 ml). The organic phase was concentrated under reduced pressure to give a residue. The residue was purified by column chromatography (SiO₂, petroleum ether/MTBE=100/1 to 50/1) to give Compound 2-2 (51.0 g, 267 mmol, 40.7% yield) as colorless oil. ¹HNMR: ET60053-22-P1A3 (400 MHz, CDCl₃) δ 3.77-3.83 (m, 4H), 1.74-1.79 (m, 2H), 0.89 (s, 9H), 0.07 (s, 6H).

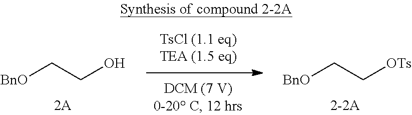 [SMILES: "TsCl (1.1 eq)TEA (1.5 eq)" ; OCCOCc1ccccc1 ; Cc1ccc(S(=O)(=O)OCCOCc2ccccc2)cc1]

To a solution of compound 2A (230 g, 1.51 mol, 215 mL, 1 eq) and TEA (229 g, 2.27 mol, 316 mL, 1.5 eq) in THF (1.5 L) was added TsCl (317 g, 1.66 mol, 1.1 eq) in portions at 0 °C maintaining internal temperature below 20 °C. After the addition, the reaction was stirred at 20 °C for 12 hrs. TLC (petroleum ether/ethyl acetate=5/1, Rf of compound 2A=0.4, Rf of compound 2-2A=1.0) showed the starting material was consumed completely. The reaction was poured into water (1.0 L). The resulting solution was extracted with MTBE (500 mL×3). The combined organic phase was washed with brine (1.5 L), dried over anhydrous Na₂SO₄ and concentrated under vacuum to give the crude product as brown solid. The crude product was triturated with a solution of petroleum ether/MTBE (2/1, 600 mL) for 1 hr at 20 °C. The resulting suspension was filtered and the filter cake was rinsed with MTBE (100 mL). The filter cake was dried under vacuum to give compound 2-2A (440 g, 1.44 mol, 95.0% yield) as off-white solid. ¹H NMR: ET61271-17-P1A, (400 MHz, CDCl₃) δ 7.78 (d, J=8.4 Hz, 2H), 7.22-7.35 (m, 7H), 4.47 (s, 2H), 4.18 (t, J=4.8 Hz, 2H), 3.65 (t, J=4.8 Hz, 2H), 2.42 (s, 3H).

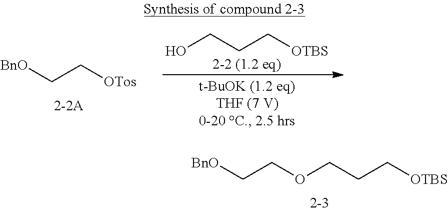

To a solution of compound 2-2 (29.8 g, 156 mmol, 1.20 eq) in THF (280 mL) was added t-BuOK (17.5 g, 156 mmol, 1.20 eq) at 0 °C under N₂ atmosphere maintaining internal temperature below 20 °C. After the addition, the reaction solution was stirred at 20 °C for 0.5 hr. Then compound 2-2A (40.0 g, 130 mmol, 1.0 eq) was added into the reaction solution dropwise at 20 °C. The resulting solution was stirred at 20 °C for 2 hrs. TLC (petroleum ether/ethyl acetate=5/1, R_{f} of compound 2-2A=0.30, and R_{f} of compound 2-3=0.60) indicated ~20% of compound 2-2A remained, and two new major spots with lower polarity were detected. The reaction was washed by saturated aqueous solution of NH₄Cl (500 ml), and the organic phase was washed by half-saturated aqueous solution NaCl (500 ml). Subsequently, the organic phase was concentrated under reduced pressure to give compound 2-3 (45.0 g, crude) as colorless oil, which was used for the next step without purification.

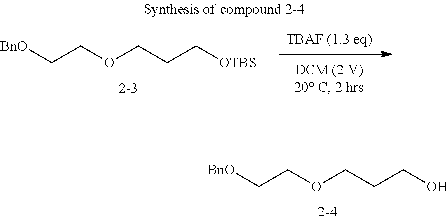 [SMILES: "TBAF (1.3 eq)" ; CC(C)(C)[Si](C)(C)OCCCOCCOCc1ccccc1 ; OCCCOCCOCc1ccccc1]

To a solution of compound 2-3 (180 g, 554 mmol, 1.00 eq) in THF (300 mL) was added TBAF (1 M, 721 mL, 1.30 eq) at 20 °C. The reaction solution was stirred at 20 °C for 2 hrs. TLC (petroleum ether/ethyl acetate=5:1, R_{f} of compound 2-3=0.9, and R_{f} of compound 2-4=0.5) indicated none of compound 2-3 remained, and one new major spot with larger polarity was detected. The reaction solution was poured into saturated aqueous solution of NH₄Cl (600 ml), and the organic phase was washed by half-saturated aqueous solution of NaCl (300 ml). The final organic was concentrated under reduced pressure to give a residue. The residue was purified by column chromatography (SiO₂, petroleum ether/ethyl acetate=400/1 to 300/1) to give compound 2-4 (45 g, 214 mmol, 38.5% yield) as brown oil. ¹HNMR: ET60053-40-P1A1 (400 MHz, CDCl₃) δ 7.34-7.38 (m, 5H), 4.57 (s, 2H), 3.77-3.80 (m, 2H), 3.63-3.70 (m, 6H), 1.85-1.88 (m, 2H).

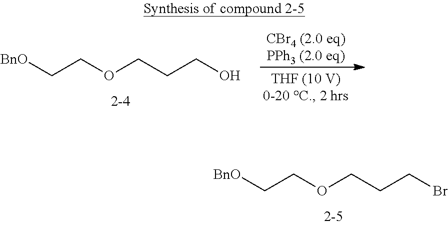 [SMILES: "CBr4 (2.0 eq)PPh3 (2.0 eq)" ; OCCCOCCOCc1ccccc1 ; BrCCCOCCOCc1ccccc1]

To a solution compound 2-4 (45.0 g, 214 mmol, 1.00 eq) in THF (225 mL) was added CBr₄ (141 g, 428 mmol, 2.0 eq). Then a solution of PPh₃ (112 g, 428 mmol, 2.00 eq) in THF (500 mL) was added into the reaction solution at 0 °C under N₂ atmosphere maintaining internal temperature below 15 °C. The resulting solution was stirred at 20 °C for 2 hrs. TLC (petroleum ether/ethyl acetate=10:1, R_{f} of compound 2-4=0.10, and R_{f} of compound 2-5=0.50) indicated none of compound 2-4 remained, and one new major spot with lower polarity was detected. The reaction solution was added into saturated aqueous solution of NH₄Cl (1000 ml), and the organic phase was washed by half-saturated aqueous solution of NaCl (500 ml). The final organic phase was concentrated under reduced pressure to give a residue. The residue was purified by column chromatography (SiO₂, petroleum ether/ethyl acetate=100/1 to 20/1) to give compound 2-5 (50.0 g, 183 mmol, 85.5% yield) as brown oil. ¹HNMR: ET60053-42-P1A4 (400 MHz, CDCl₃) δ 7.29-7.36 (m, 5H), 4.58 (s, 2H), 3.60-3.65 (m, 6H), 3.51-3.54 (t, J=6.8 Hz, 2H), 2.01-2.16 (m, 2H).

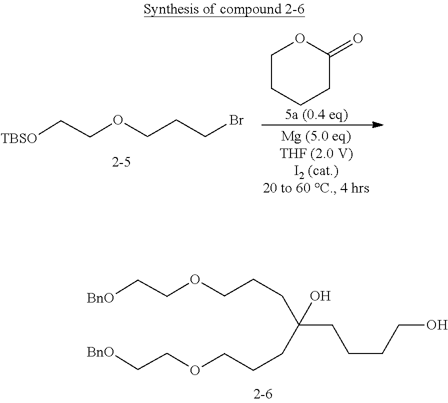 [SMILES: O=C1CCCCO1 ; CC(C)(C)[Si](C)(C)OCCOCCCBr ; OCCCCC(O)(CCCOCCOCc1ccccc1)CCCOCCOCc1ccccc1]

To a suspension of Mg (5.34 g, 220 mmol, 5 eq) in THF (50 mL) was added I2 (112 mg, 439 umol, 0.01 eq) in one portion at 25 °C. The resulting suspension was stirred at 25 °C for 1 hr. Then the suspension was heated to 50 °C and compound 2-5 (12 g, 43.9 mmol, 1 eq) was dropwise added into the reaction at 50 °C maintaining internal temperature below 60 °C. After the addition, the resulting suspension was stirred at 25 °C for 1 h. Then compound 5a (1.76 g, 17.6 mmol, 0.4 eq) was added into the reaction in portions at 25 °C. The reaction was stirred at 25 °C for 2 hrs. TLC (petroleum ether/ethyl acetate=10/1, R_{f} of compound 2-5=0.6, R_{f} of compound 2-6=0.0) showed the starting material was consumed completely. The reaction was poured into aqueous solution of NH₄Cl (Sat. 30 mL). The resulting suspension was extracted with MTBE (20 mL×3). The combined organic phase was washed with brine (30 mL) and concentrated under vacuum to give the crude product as brown oil. The crude product was purified by column chromatography on silica gel (petroleum ether: ethyl acetate=10:1 to 3:1) to give compound 2-6 (4.5 g, 9.21 mmol, 21.0% yield) as light yellow oil. ¹H NMR: ET61271-26-P1A, (400 MHz, CDCl₃) δ 7.27-7.37 (m, 10H), 4.57 (s, 4H), 3.59-3.62 (m, 10H), 3.49 (t, J=6.0 Hz, 4H), 1.38-1.68 (m, 14H).

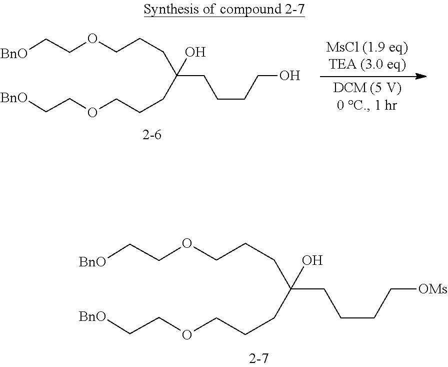 [SMILES: OCCCCC(O)(CCCOCCOCc1ccccc1)CCCOCCOCc1ccccc1 ; CS(=O)(=O)OCCCCC(O)(CCCOCCOCc1ccccc1)CCCOCCOCc1ccccc1]

To a solution of compound 2-6 (6.00 g, 12.3 mmol, 1.00 eq) in DCM (30 mL) was added TEA (3.73 g, 36.8 mmol, 5.13 mL, 3.00 eq) and MsCl (2.67 g, 23.3 mmol, 1.80 mL, 1.90 eq) at 0 °C. The solution was stirred at 0 °C for 1 h. TLC (petroleum ether/ethyl acetate=0/1, R_{f} of compound 2-6=0.47, R_{f} of compound 2-7=0.65) showed the reaction was completed. The reaction solution was poured into water (50 mL). The aqueous layer was extracted with DCM (30 mL×2). The combined organic layer was dried over Na₂SO₄, filtered and concentrated under vacuum to give a residue. Compound 2-7 (7.00 g, crude) was obtained as yellow oil, which was used directly to the next step without purification. ¹H NMR: ET60190-19-P1A (400 MHz, CDCl₃) δ 7.28-7.35 (m, 10H), 4.56 (s, 4H), 4.18-4.24 (m, 2H), 3.62 (s, 8H), 3.43-3.50 (m, 4H), 2.98 (s, 3H), 1.35-1.83 (m, 14H).

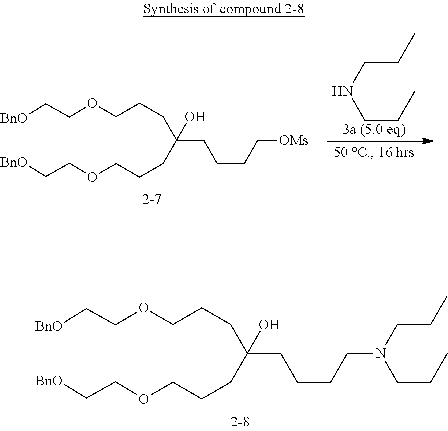

A solution of compound 2-7 (6.00 g, 10.6 mmol, 1.00 eq) and compound 3a (5.36 g, 52.9 mmol, 7.30 mL, 5.00 eq) was stirred at 50 °C for 16 hrs. TLC (petroleum ether/ethyl acetate=0/1, R_{f} of compound 2-7=0.69, R_{f} of compound 2-8=0.03) showed the reaction was completed. The reaction solution was concentrated under vacuum to give a residue. The residue was purified by column chromatography (SiO₂, DCM/MeOH=500/1 to 5/1) to give compound 2-8 (4.00 g, 7.00 mmol, 66.1% yield) as yellow oil. ¹H NMR: ET60190-20-P1A2 (400 MHz, CDCl₃) δ 7.28-7.35 (m, 10H), 4.58 (s, 4H), 3.62 (s, 8H), 3.44-3.50 (m, 4H), 2.36-2.45 (m, 6H), 1.60-1.66 (m, 6H), 1.44-1.53 (m, 10H), 1.26-1.28 (m, 2H), 0.88 (t, J=7.2 Hz, 6H).

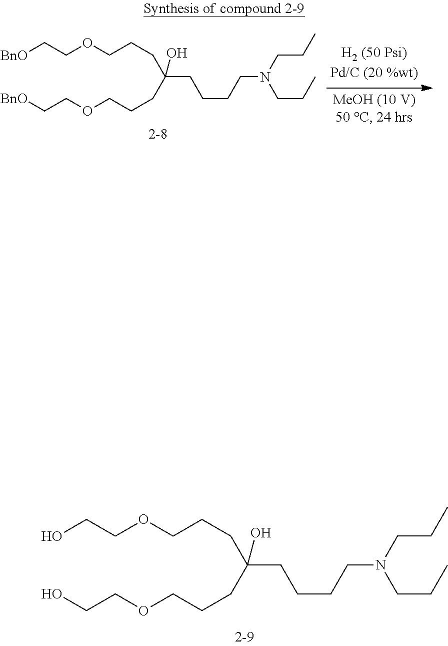 [SMILES: CCCN(CCC)CCCCC(O)(CCCOCCOCc1ccccc1)CCCOCCOCc1ccccc1 ; CCCN(CCC)CCCCC(O)(CCCOCCO)CCCOCCO]

A suspension of compound 2-8 (4.00 g, 7.00 mmol, 1.00 eq) and Pd/C (1 g, 10% purity) in MeOH (50 mL) was degassed under vacuum and purged with H2 (50 psi) for three times. The suspension was stirred under H2 (50 psi) at 50 °C for 24 hours. LCMS showed the reaction was completed. The reaction suspension was filtered and the filtrate was concentrated under vacuum to give compound 2-9 (2.50 g, 6.38 mmol, 91.3% yield) as yellow oil, which was used directly to the next step without purification. LCMS: ET60190-21-P1A2 (RT=0.381 min, M+H⁺=392.2). ¹H NMR: ET60190-21-P1A2 (400 MHz, CDCl₃) δ 3.72 (t, J=4.4 Hz, 4H), 3.52-3.56 (m, 4H), 3.46-3.51 (m, 4H), 2.34-2.39 (m, 6H), 1.60-1.64 (m, 4H), 1.51-1.55 (m, 2H), 1.42-1.48 (m, 8H), 1.24-1.27 (m, 4H), 0.86 (t, J=7.2 Hz, 6H).

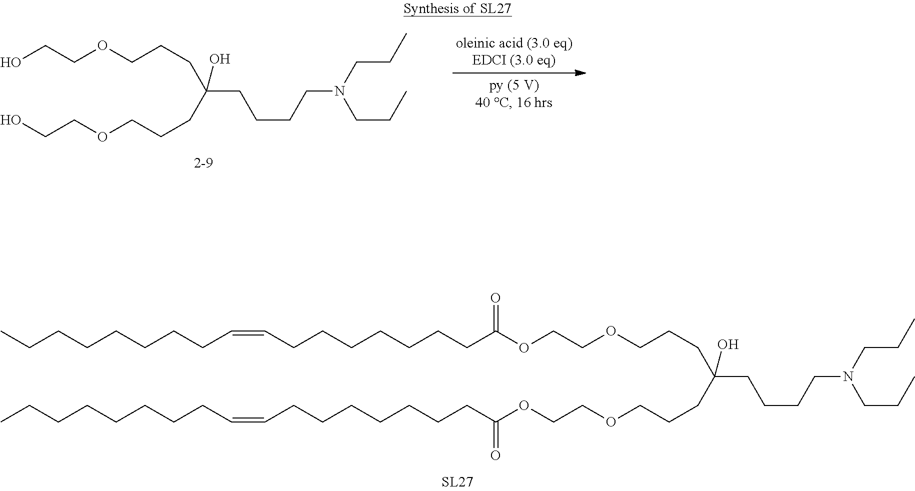 [SMILES: CCCN(CCC)CCCCC(O)(CCCOCCO)CCCOCCO ; CCCCCCCC/C=C\CCCCCCCC(=O)OCCOCCCC(O)(CCCCN(CCC)CCC)CCCOCCOC(=O)CCCCCCC/C=C\CCCCCCCC]

To a solution of compound 2-9 (2.00 g, 5.11 mmol, 1.00 eq) and oleic acid (4.33 g, 15.3 mmol, 4.33 mL, 3.00 eq) in Py (10 mL) was added EDCI (2.94 g, 15.3 mmol, 3.00 eq). The solution was stirred at 40 °C for 16 hrs. LCMS showed the reaction was completed. The reaction solution was concentrated under vacuum to give a residue. The residue was poured into saturated aqueous solution of NH₄Cl (100 mL), and the aqueous layer was extracted with EtOAc (50 mL×3). The combined organic layer was concentrated under vacuum to give a residue. The residue was purified by column chromatography (SiO₂, petroleum ether/ethyl acetate=10/1 to 0/1) to give SL27 (1.00 g, 1.09 mmol, 21.3% yield) as yellow oil. LCMS: ET60190-23-P1A2 (RT=2.831 min, M+H⁺=921.0). ¹H NMR: ET60190-23-P1A06 (400 MHz, CDCl₃) δ 5.33-5.37 (m, 4H), 4.22 (t, J=4.8 Hz, 4H), 3.64 (t, J=5.2 Hz, 4H), 3.48 (t, J=6.0 Hz, 4H), 2.80-2.86 (m, 6H), 2.34 (t, J=7.6 Hz, 4H), 1.60-1.64 (m, 14H), 1.48-1.52 (m, 8H), 1.25-1.32 (m, 40H), 0.99 (t, J=6.8 Hz, 6H), 0.89 (t, J=6.8 Hz, 6H).

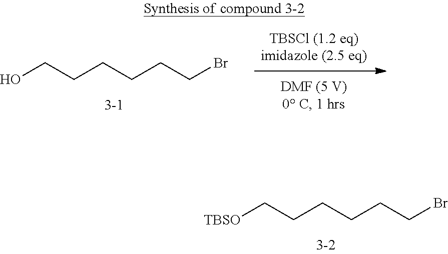 [SMILES: "TBSCl (1.2 eq)imidazole (2.5 eq)" ; OCCCCCCBr ; CC(C)(C)[Si](C)(C)OCCCCCCBr]

To a solution of compound 3-1 (90.0 g, 497 mmol, 65.2 mL, 1.00 eq) and imidazole (84.5 g, 1.24 mol, 2.50 eq) in DMF (200 mL) was added a solution of TBSCl (89.9 g, 596 mmol, 73.1 mL, 1.20 eq) in DMF (250 mL) dropwise at 0 °C. The solution was stirred at 0 °C under nitrogen protection another 1 h. TLC (petroleum ether/ethyl acetate=10:1, R_{f} of compound 3-1=0.48, R_{f} of compound 3-2=0.72) indicated reactant was consumed completely and one new spot with lower polarity was detected. The reaction solution was poured into saturated aqueous solution of NH₄Cl (550 mL). The solution was extracted with MTBE (500 mL) for three times. The combined organic layer was dried over sodium sulfate, filtered and concentrated under reduced pressure to give a residue. The crude product was purified by column chromatography (SiO₂, petroleum ether/ethyl acetate=1/0 to 50/1) to give compound 3-2 (143 g, 487 mmol, 98.0% yield) as colorless oil. ¹H NMR: ET60052-3-P1A (400 MHz, CDCl₃) δ 3.61 (t, J=6.4 Hz, 2H), 3.41 (t, J=7.2 Hz, 2H), 1.91-1.84 (m, 2H), 1.56-1.51 (m, 2H), 1.49-1.42 (m, 2H), 1.40-1.34 (m, 2H), 0.90 (s, 9H), 0.05 (s, 6H).

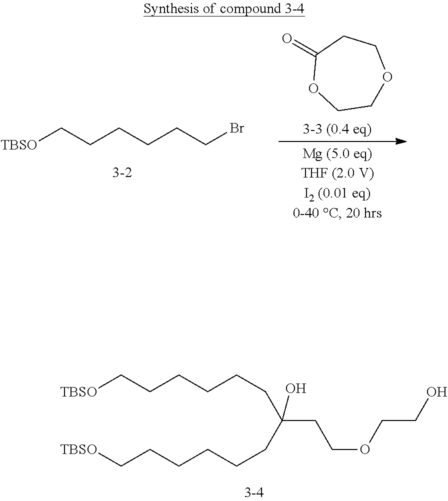 [SMILES: O=C1CCOCCO1 ; CC(C)(C)[Si](C)(C)OCCCCCCBr ; CC(C)(C)[Si](C)(C)OCCCCCCC(O)(CCCCCCO[Si](C)(C)C(C)(C)C)CCOCCO]

To a suspension of Mg (32.9 g, 1.35 mol, 5.00 eq) in THF (80 mL) was added 12 (688 mg, 2.71 mmol, 546 μL, 0.01 eq) in one portion under N₂ at 20 °C. The suspension was stirred at 40 °C for 1 h. Then a solution of compound 3-2 (80.0 g, 271 mmol, 1.00 eq) in THF (80 mL) was added dropwise to the above suspension at 40 °C. The suspension was stirred at 40 °C for 3 hrs. Then compound 3-3 (12.6 g, 108 mmol, 318 μL, 0.40 eq) was added dropwise to the suspension at 0 °C. The suspension was stirred at 40 °C for 16 hrs. TLC (petroleum ether/ethyl acetate=10/1, R_{f} of compound 3-2=0.61, R_{f} of compound 3-4=0.03) showed the reaction was completed. The reaction solution was poured into a saturated aqueous solution of NH₄Cl (300 mL). The aqueous layer was extracted with EtOAc (200 mL×3). The combined organic layer was dried over Na₂SO₄, filtered and concentrated under vacuum to give a residue. The residue was purified by column chromatography (SiO₂, petroleum ether/ethyl acetate=800/1 to 10/1) to give compound 3-4 (50.0 g, 91.1 mmol, 67.2% yield) as colorless oil. ¹H NMR: ET60190-8-P1A (400 MHz, CDCl₃) δ 3.75 (s, 2H), 3.68-3.73 (m, 2H), 3.57-3.65 (m, 6H), 2.16 (s, 2H), 1.77 (t, J=6.0 Hz, 2H), 1.44-1.54 (m, 8H), 1.26-1.33 (m, 12H), 0.93 (s, 18H), 0.05 (s, 12H).

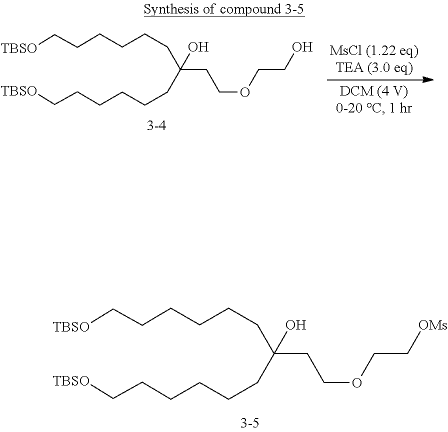 [SMILES: CC(C)(C)[Si](C)(C)OCCCCCCC(O)(CCCCCCO[Si](C)(C)C(C)(C)C)CCOCCO ; CC(C)(C)[Si](C)(C)OCCCCCCC(O)(CCCCCCO[Si](C)(C)C(C)(C)C)CCOCCOS(C)(=O)=O]

To a solution of compound 3-4 (25.0 g, 45.5 mmol, 1.00 eq) in DCM (100 mL) was added TEA (13.8 g, 137 mmol, 19.0 mL, 3.00 eq) and MsCl (6.39 g, 55.8 mmol, 4.32 mL, 1.22 eq) at 0 °C. The solution was stirred at 20 °C for 1 hr. TLC (petroleum ether/ethyl acetate=2/1, R_{f} of compound 3-4=0.38, R_{f} of compound 3-5=0.48) showed the reaction was completed. The reaction solution was poured into NH₄Cl (Sat. aq. 200 mL). The aqueous layer was extracted with DCM (100 mL×2). The combined organic layer was dried over Na₂SO₄, filtered and concentrated under vacuum to give compound 3-5 (26.0 g, crude) as light yellow oil, which was used directly to the next step. ¹H NMR: ET60190-13-P1A (400 MHz, CDCl₃) δ 4.35-4.38 (m, 2H), 3.70-3.74 (m, 4H), 3.61 (t, J=6.8 Hz, 4H), 3.06 (s, 3H), 1.78 (t, J=6.0 Hz, 2H), 1.48-1.54 (m, 4H), 1.42-1.45 (m, 4H), 1.26-1.33 (m, 12H), 0.90 (s, 18H), 0.06 (s, 12H).

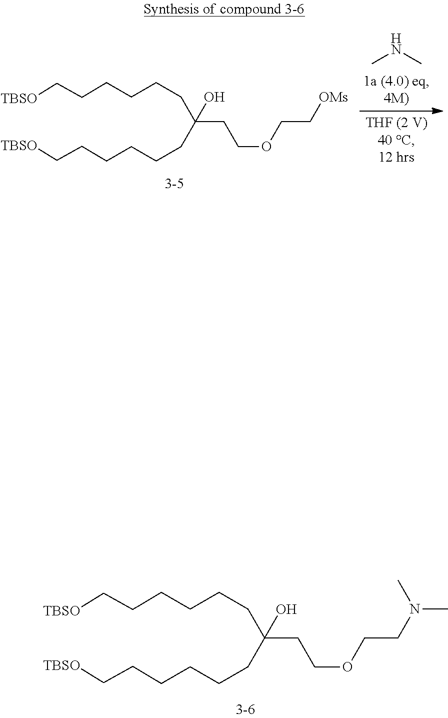

A solution of compound 3-5 (10.0 g, 15.9 mmol, 1.00 eq) and compound 1a (2 M, 31.8 mL, 4.00 eq) was stirred at 40 °C for 12 hrs in a 100 mL of sealed tube. TLC (petroleum ether/ethyl acetate=1/1, R_{f} of compound 3-5=0.87, R_{f} of compound 3-6=0.02) showed the starting material was consumed completely. The reaction was concentrated under reduced pressure to give a residue. The residue was purified by column chromatography (SiO₂, petroleum ether/ethyl acetate=400/1 to 0/1) to give compound 3-6 (7.70 g, 13.3 mmol, 83.8% yield) as light yellow oil. ¹H NMR: ET60044-12-P1A (400 MHz, CDCl₃) δ 3.66-6.52 (m, 8H), 2.49 (t, J=5.6 Hz, 2H), 2.25 (s, 6H), 1.72 (t, J=5.6 Hz, 2H), 1.57-1.53 (m, 8H), 1.30-1.26 (m, 12H), 0.89 (s, 18H), 0.04 (s, 12H).

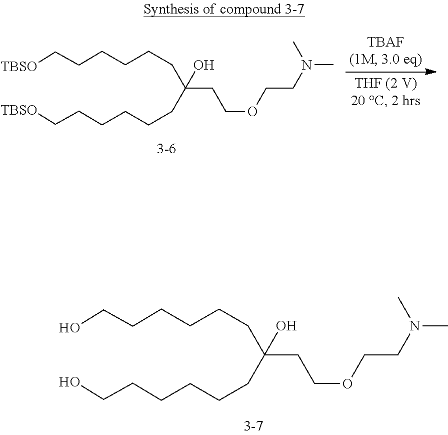 [SMILES: CN(C)CCOCCC(O)(CCCCCCO[Si](C)(C)C(C)(C)C)CCCCCCO[Si](C)(C)C(C)(C)C ; CN(C)CCOCCC(O)(CCCCCCO)CCCCCCO]

To a solution of compound 3-6 (7.0 g, 12.1 mmol, 1.00 eq) in THF (14 mL) was added TBAF (1 M, 36.4 mL, 3.00 eq), and the solution was stirred at 20 °C for 2 h. TLC (DCM/MeOH=10/1, R_{f} of compound 3-6=0.34, R_{f} of compound 3-7=0.34) showed the starting material was consumed completely. The reaction solution was concentrated under reduced pressure to give compound 3-7 (4.50 g, crude) as brown oil.

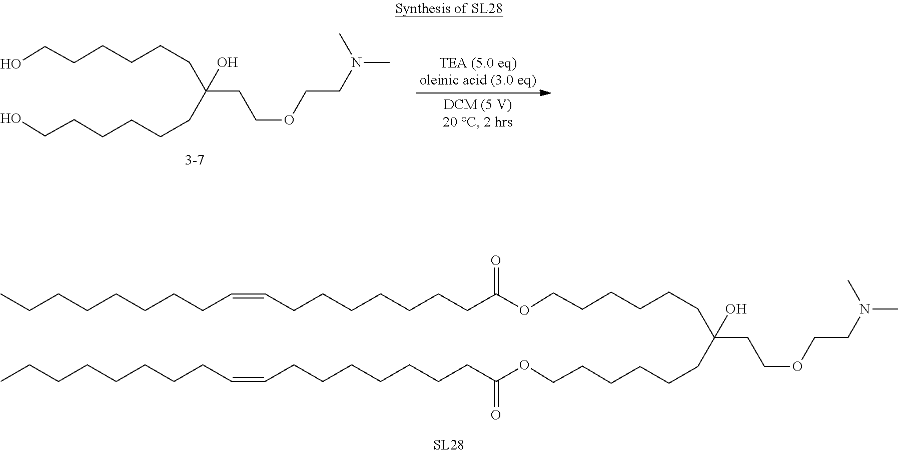 [SMILES: CN(C)CCOCCC(O)(CCCCCCO)CCCCCCO ; CCCCCCCC/C=C\CCCCCCCC(=O)OCCCCCCC(O)(CCCCCCOC(=O)CCCCCCC/C=C\CCCCCCCC)CCOCCN(C)C]

To a solution of compound 3-7 (4.50 g, 12.9 mmol, 1.00 eq) in Py (23 mL) was added oleic acid (20.1 g, 71.2 mmol, 20.1 mL, 5.50 eq). EDCI (12.4 g, 64.7 mmol, 5.00 eq) was added into the solution. The solution was stirred at 45 °C for 12 h. LCMS showed the reaction was finished. The reaction solution was poured into water (150 mL), the aqueous phase was extracted with EtOAc (50 mL×2). The combined organic phase was washed with brine (100 mL), dried with anhydrous Na₂SO₄, filtered and concentrated in vacuum. The residue was purified by column chromatography (SiO₂, petroleum ether/ethyl acetate=100/1 to 0/1) to give a crude product, and the crude product was purified by prep-HPLC (column: Welch Xtimate C1 100*30 mm*5 um; mobile phase: [water (FA)-ACN]; B %: 50%-85%, 20 min) to give SL28 (1.0 g, 1.14 mmol, 8.81% yield) as yellow oil. LCMS: ET60044-18-P1A (RT=2.942 min, M+Na⁺=898.80). ¹H NMR: ET60044-18-P1A (400 MHz, CDCl₃) δ 5.36-5.33 (m, 4H), 4.05 (t, J=6.4 Hz, 4H), 3.66-3.62 (m, 4H), 3.12 (br.s, 1H), 2.68-2.66 (m, 2H), 2.40 (s, 6H), 2.29 (t, J=7.2 Hz, 4H), 2.01-1.98 (m, 7.2 Hz, 8H), 1.74 (t, J=5.6 Hz, 2H), 1.62-1.60 (m, 8H), 1.55-1.45 (m, 4H), 1.30-1.27 (m, 52H), 0.88 (t, J=8.0 Hz, 6H).

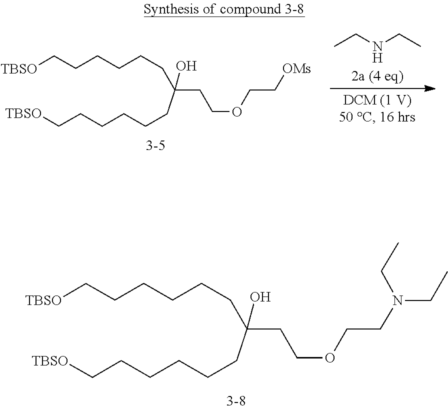

A solution of compound 3-5 (10.0 g, 16.0 mmol, 1.00 eq) and compound 2a (5.83 g, 79.7 mmol, 8.21 mL, 5.00 eq) in DCM (10 mL) was stirred at 50 °C for 16 h. TLC (petroleum ether/ethyl acetate=2/1, R_{f} of compound 3-5=0.56, R_{f} of compound 3-8=0.03) showed the reaction was completed. The reaction solution was concentrated under vacuum to give a residue. The residue was purified by column chromatography (SiO₂, DCM/MeOH=1/0 to 5/1) to give compound 3-8 (7.20 g, 11.9 mmol, 74.7% yield) as light yellow oil. ¹H NMR: ET60190-15-P1A (400 MHz, CDCl₃) δ 3.54-3.65 (m, 8H), 2.58-3.67 (m, 6H), 1.73 (s, 2H), 1.25-1.52 (m, 20H), 1.03-1.06 (m, 6H), 0.90 (s, 18H), 0.05 (s, 12H).

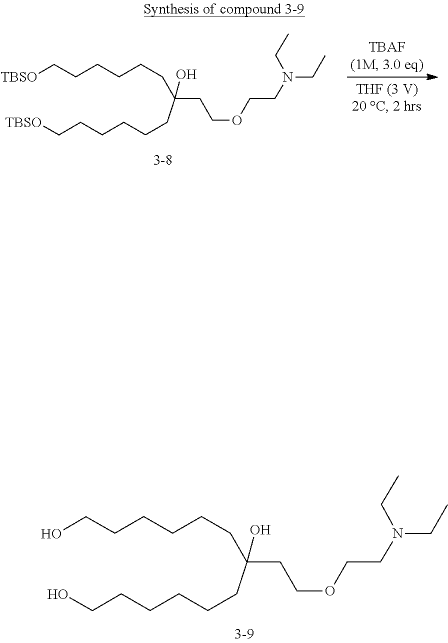 [SMILES: CCN(CC)CCOCCC(O)(CCCCCCO[Si](C)(C)C(C)(C)C)CCCCCCO[Si](C)(C)C(C)(C)C ; CCN(CC)CCOCCC(O)(CCCCCCO)CCCCCCO]

To a solution of compound 3-8 (6.50 g, 10.7 mmol, 1.00 eq) in THF (18.0 mL) was added TBAF (1 M, 32.2 mL, 3.00 eq) dropwise at 20 °C. The solution was stirred at 20 °C under N₂ atmosphere for 2 h. TLC (petroleum ether/ethyl acetate=10/1, R_{f} of compound 3-8=0.52, R_{f} of compound 3-9=0.20) indicated reactant was consumed completely and one new spot with higher polarity was detected. The reaction solution was poured into water (100 mL). The solution was extracted with EtOAc (150 mL×3). The combined organic layer was dried over sodium sulfate, filtered and concentrated under reduced pressure to give the crude product compound 3-9 (4 g, crude) as colorless oil which was used into the next step without further purification.

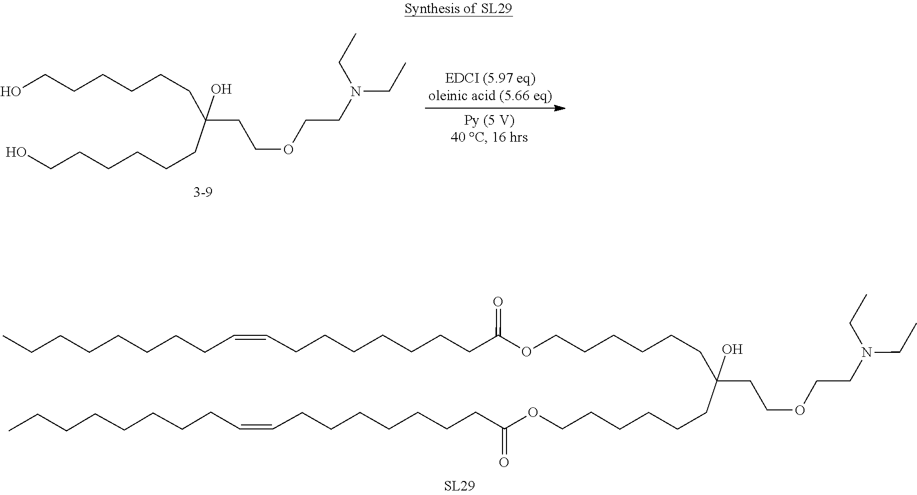 [SMILES: CCN(CC)CCOCCC(O)(CCCCCCO)CCCCCCO ; CCCCCCCC/C=C\CCCCCCCC(=O)OCCCCCCC(O)(CCCCCCOC(=O)CCCCCCC/C=C\CCCCCCCC)CCOCCN(CC)CC]

To a solution of compound 3-9 (4.00 g, 10.6 mmol, 1.00 eq) and oleic acid (17.0 g, 60.2 mmol, 5.66 eq) in Py (20.0 mL) was added a solution of EDCI (12.1 g, 63.5 mmol, 5.97 eq) dropwise at 40 °C. The solution was stirred at 40 °C under nitrogen protection another 16 hrs. LCMS showed none of compound 3-9 remained. Several new peaks were shown on LCMS and 60% of desired compound SL29 was detected. The reaction solution was poured into water (150 mL). The solution was extracted with EtOAc (300 mL×3). The crude product was purified by column chromatography (SiO₂, DCM/MeOH=1/0 to 10/1) to give compound SL29 (1.00 g, 1.11 mmol, 10.4% yield) as colorless oil. LCMS: ET60052-23-P1A3 (RT=3.854 min, M+H⁺=904.8). ¹H NMR: ET60052-23-P1A (400 MHz, CDCl₃) δ 5.36-5.33 (m, 4H), 4.05 (d, J=6.0 Hz, 4H), 3.65 (d, J=6.0 Hz, 2H), 3.60 (s, 2H), 2.71 (s, 2H), 2.65 (s, 4H), 2.29 (d, J=8.0 Hz, 4H), 2.03-1.99 (m, 8H), 1.72 (d, J=6.0 Hz, 2H), 1.63-1.61 (m, 8H), 1.47-1.27 (m, 57H), 1.07 (d, J=8.0 Hz, 6H), 0.88 (d, J=6.0 Hz, 6H).

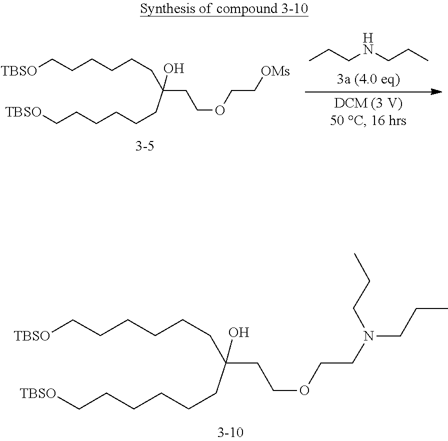

A solution of compound 3-5 (4.40 g, 7.02 mmol, 1.00 eq) and compound 3a (4.26 g, 42.1 mmol, 5.80 mL, 6.00 eq) in DCM (12.0 mL) was stirred at 50 °C under N₂ atmosphere for 16 hrs. LCMS showed 20% of compound 3-5 remained. Several new peaks were shown on LCMS and 50% of desired compound was detected. The reaction solution was poured into water (150 mL). The solution was extracted with EtOAc (300 mL×3). The combined organic layer was dried over sodium sulfate, filtered and concentrated under reduced pressure to give the crude product. The crude product was purified by column chromatography (SiO₂, DCM/MeOH=1/0 to 10/1) to give compound 3-10 (3.02 g, 4.78 mmol, 68.1% yield) as colorless oil. LCMS: ET60052-12-P1A1 (RT=1.030 min, M+H⁺=632.54). ¹H NMR: ET60052-12-P1A (400 MHz, CDCl₃) δ 3.66-3.58 (m, 6H), 3.51 (s, 2H), 2.64 (s, 2H), 2.43 (m, 4H), 1.72 (t, J=5.8 Hz, 2H), 1.53-1.47 (m, 12H), 1.30 (s, 12H), 0.89 (s, 18H), 0.87 (t, J=9.0 Hz, 6H), 0.05 (s, 12H).

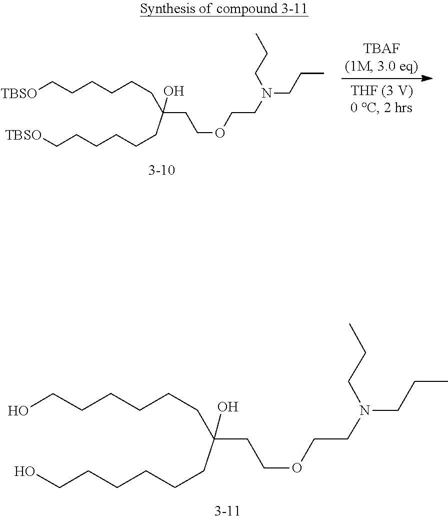 [SMILES: CCCN(CCC)CCOCCC(O)(CCCCCCO[Si](C)(C)C(C)(C)C)CCCCCCO[Si](C)(C)C(C)(C)C ; CCCN(CCC)CCOCCC(O)(CCCCCCO)CCCCCCO]

To a solution of compound 3-10 (2.80 g, 4.43 mmol, 1.00 eq) in THF (9.00 mL) was added TBAF (1 M, 13.3 mL, 3.00 eq) dropwise at 0 °C. The solution was stirred at 0 °C under N₂ atmosphere 2 hrs. TLC (petroleum ether/ethyl acetate=10:1, R_{f} of compound 3-11=0.52, R_{f} of compound 3-11=0.2) indicated reactant was consumed completely and one new spot with higher polarity was detected. The reaction solution was poured into water (100 mL). The solution was extracted with EtOAc (150 mL×3). The combined organic layer was dried over sodium sulfate, filtered and concentrated under reduced pressure to give a residue. The crude product was purified by column chromatography (SiO₂, DCM/MeOH=1/0 to 10/1) to give compound 3-11 (1.30 g, 3.22 mmol, 72.7% yield) as colorless oil. ¹H NMR: ET60052-13-P1A1 (400 MHz, CDCl₃) δ 3.65-3.62 (m, 6H), 3.51 (s, 2H), 2.64 (s, 2H), 2.42 (s, 4H), 1.72 (t, J=6.0 Hz, 2H), 1.61-1.54 (m, 5H), 1.50-1.27 (m, 21H), 0.85 (t, J=7.4 Hz, 6H).

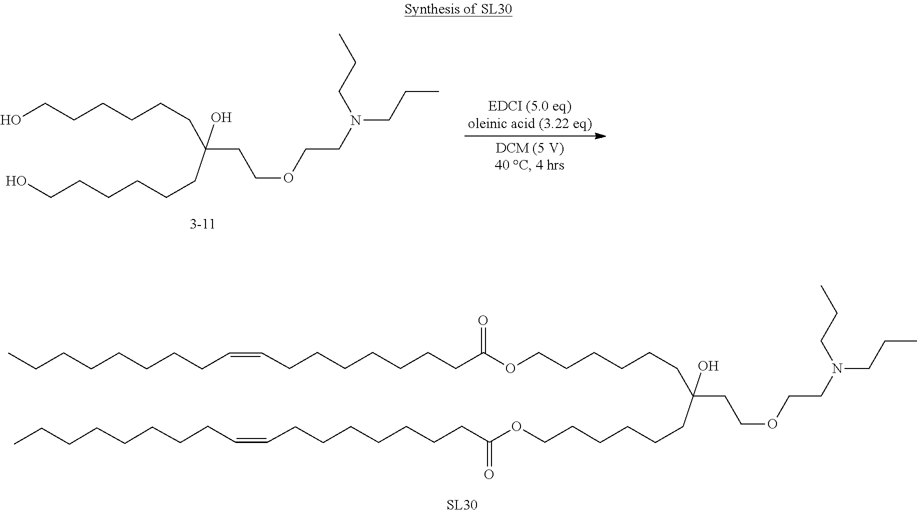 [SMILES: CCCN(CCC)CCOCCC(O)(CCCCCCO)CCCCCCO ; CCCCCCCC/C=C\CCCCCCCC(=O)OCCCCCCC(O)(CCCCCCOC(=O)CCCCCCC/C=C\CCCCCCCC)CCOCCN(CCC)CCC]

To a solution of compound 3-11 (1.00 g, 2.48 mmol, 1.00 eq) and oleic acid (2.25 g, 7.97 mmol, 2.25 mL, 3.22 eq) in Py (3.00 mL) was added EDCI (2.37 g, 12.4 mmol, 5.00 eq) at 40 °C. The solution was stirred at 40 °C under nitrogen protection another 4 hrs. LCMS showed none of compound 3-11 remained. Several new peaks were shown on LCMS and 80% of desired SL30 was detected. The reaction solution was poured into water (150 mL). The solution was extracted with (300 mL×3). The combined organic layer was dried over sodium sulfate, filtered and concentrated under reduced pressure to give a residue. The crude product was purified by column chromatography (SiO₂, DCM/MeOH=1/0 to 10/1) to give crude of SL30 (1.80 g) as colorless oil. The crude product was purified by column chromatography (SiO₂, petroleum ether/ethyl acetate=100/1 to 5/1) to give SL30 (1.00 g, 1.07 mmol, 43.3% yield) as colorless oil. LCMS: ET60052-18-P1A3 (RT=3.985 mins, M+H⁺=932.86). ¹H NMR: ET60052-19-P1A3 (400 MHz, CDCl₃) δ 5.36-5.33 (m, 4H), 4.05 (d, J=6.8 Hz, 4H), 3.64 (d, J=5.6 Hz, 2H), 3.52 (s, 2H), 2.66 (s, 2H), 2.65 (s, 4H), 2.29 (d, J=7.4 Hz, 4H), 2.02-1.99 (m, 8H), 1.72 (d, J=5.8 Hz, 2H), 1.62-1.55 (m, 8H), 1.47-1.27 (m, 62H), 0.89-0.86 (m, 12H).

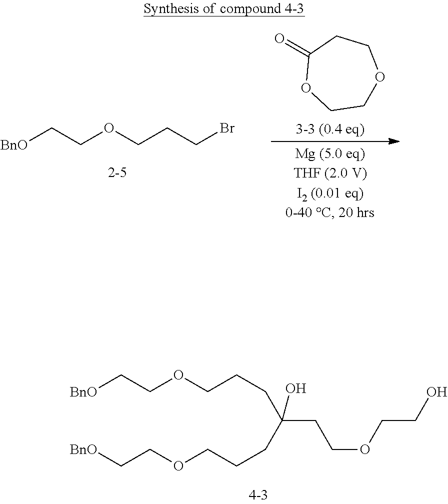 [SMILES: O=C1CCOCCO1 ; BrCCCOCCOCc1ccccc1 ; OCCOCCC(O)(CCCOCCOCc1ccccc1)CCCOCCOCc1ccccc1]

To a suspension of Mg (22.3 g, 915 mmol, 5.00 eq) in THF (50 mL) was added 12 (465 mg, 1.83 mmol, 369 μL, 0.01 eq) in one portion under N₂ at 20 °C. The suspension was stirred at 40 °C for 1 h. Then compound 2-5 (50.0 g, 183 mmol, 1.00 eq) was added dropwise to the above suspension at 40 °C. The suspension was stirred at 40 °C for 3 h. Then compound 3-3 (8.50 g, 73.2 mmol, 0.40 eq) was added dropwise to the suspension at 0 °C. The suspension was stirred at 40 °C for 16 hrs. TLC (petroleum ether/ethyl acetate=10/1, R_{f} of compound 2-5=0.56, R_{f} of compound 4-3=0.04) showed the reaction was completed. The reaction solution was poured into a saturated aqueous solution of NH₄Cl (200 mL). The aqueous layer was extracted with EtOAc (100 mL×3). The combined organic layer was dried over Na₂SO₄, filtered and concentrated under vacuum to give a residue. The residue was purified by column chromatography (SiO₂, petroleum ether/ethyl acetate=800/1 to 0/1) to give compound 4-3 (32.0 g, 63.4 mmol, 69.3% yield) as yellow oil. ¹H NMR: ET60190-17-P1A (400 MHz, CDCl₃) (7.27-7.35 (m, 10H), 4.57 (s, 4H), 3.61-3.69 (m, 12H), 3.47-3.53 (m, 5H), 1.76-1.79 (m, 2H), 1.57-1.65 (m, 8H).

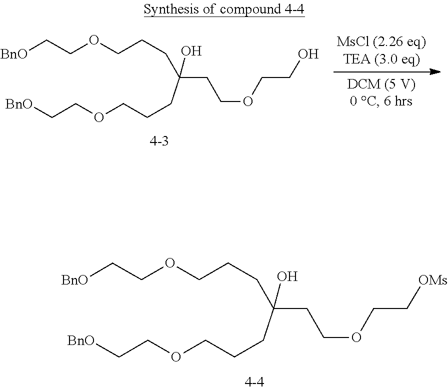 [SMILES: OCCOCCC(O)(CCCOCCOCc1ccccc1)CCCOCCOCc1ccccc1 ; CS(=O)(=O)OCCOCCC(O)(CCCOCCOCc1ccccc1)CCCOCCOCc1ccccc1]

To a solution of compound 4-3 (23.0 g, 45.6 mmol, 1.00 eq) and TEA (13.8 g, 136 mmol, 19.0 mL, 3.00 eq) in DCM (120 mL) was added dropwise MsCl (11.8 g, 103 mmol, 7.97 mL, 2.26 eq) at 0 °C. After the addition, the solution was stirred at this temperature for 6 hrs. TLC (petroleum ether/ethyl acetate=1/1, R_{f} of compound 4-3=0.26, R_{f} of compound 4-4=0.37) showed that the reaction was finished. The solution was poured into NH₄Cl (aq. sat. 200 mL). The solution was extracted with DCM (50.0 mL×2). The organic layers were combined and concentrated under vacuum to give crude product. The crude product was purified by column chromatography (SiO₂, petroleum ether/ethyl acetate=50/1 to 0/1) to give compound 4-4 (26.0 g, 44.6 mmol, 97.9% yield) as yellow oil.

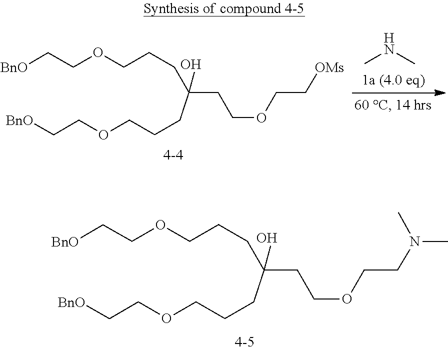

To a solution of compound 4-4 (14.1 g, 24.2 mmol, 1.00 eq) was added compound 1a (2.0 M, 48.4 mL, 4.0 eq) at 20 °C in sealed tube. The reaction solution was stirred at 60 °C for 14 hrs. TLC (ethyl acetate/MeOH=8/1, R_{f} of Compound 4-4=0.92, R_{f} of Compound 4-5=0.52) indicated none of compound 4-4 remained, and one new major spot with higher polarity was detected. The reaction solution was poured into the saturated aqueous solution of NH₄Cl (20.0 ml), and the organic phase was washed by half-saturated aqueous solution of NaCl (10 ml). The final organic phase was concentrated under reduced pressure to give crude compound 4-5 (6.70 g, 12.6 mmol, 51.9% yield) as brown oil. ¹HNMR: ET60053-52-P1A2 (400 MHz, CDCl₃) δ 7.27-7.35 (m, 10H), 4.55 (s, 4H), 3.62-3.63 (m, 4H), 3.61 (s, 2H), 3.53-3.56 (t, J=1.6 Hz, 6H), 3.45-3.48 (m, 4H), 2.53-2.55 (t, J=5.6 Hz, 2H), 2.30 (s, 6H) 1.60-1.63 (m, 2H), 1.52-1.55 (m, 8H).

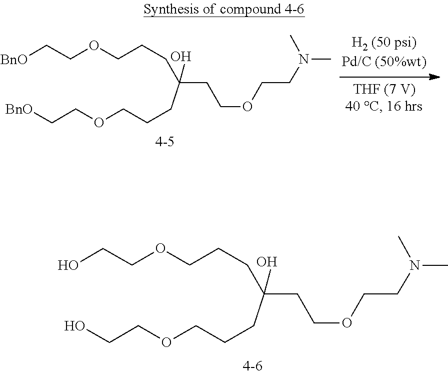 [SMILES: CN(C)CCOCCC(O)(CCCOCCOCc1ccccc1)CCCOCCOCc1ccccc1 ; CN(C)CCOCCC(O)(CCCOCCO)CCCOCCO]

To a solution of compound 4-5 (6.00 g, 11.2 mmol, 1.00 eq) in THF (40.0 mL) was added Pd/C (10.0% purity, 3.0 g) under N₂ atmosphere. The suspension was degassed and purged with H2 (50 psi) for 3 times. The mixture was stirred under H2 (50.0 psi) at 40 °C for 16 hrs. TLC (petroleum ether/ethyl acetate=1/1, R_{f} of compound 4-5=0.92, R_{f} of compound 4-6=0.41) indicated none of compound 2-8 remained, and one new major spot with higher polarity was detected. The reaction solution was filtered and the filtrate was poured into saturated aqueous solution of NH₄Cl (20 ml). The organic phase was washed by half-saturated aqueous solution of NaCl (20.0 ml) and concentrated under reduced pressure to give the crude product. The crude product was purified by column chromatography (SiO₂, petroleum ether/ethyl acetate=3/1 to 1/1) to give compound 4-6 (3.70 g, 10.5 mmol, 93.2% yield) as brown oil. ¹HNMR: ET60053-54-P1A1 (400 MHz, CDCl₃) δ 3.70-3.73 (m, 4H), 3.66 (s, 2H), 3.52-3.55 (m, 8H), 3.47-3.49 (m, 4H), 2.48-2.51 (t, J=5.6 Hz), 2.26 (s, 6H), 1.74-1.77 (t, J=6 Hz, 2H), 1.56-1.63 (m, 8H).

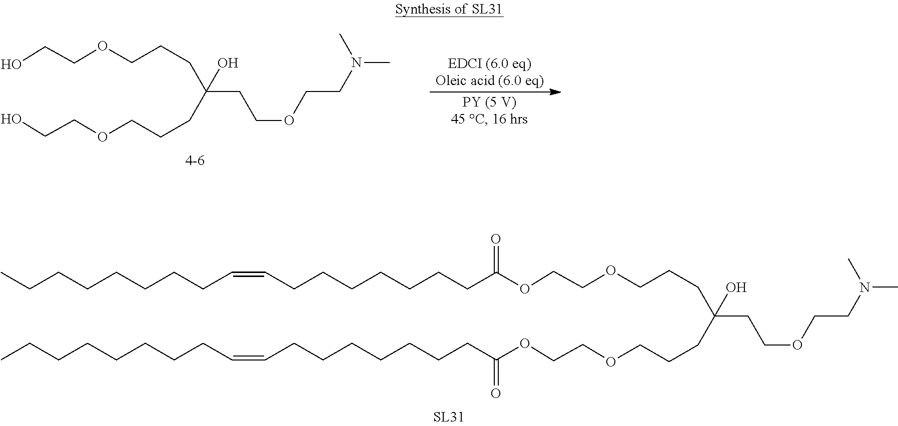 [SMILES: CN(C)CCOCCC(O)(CCCOCCO)CCCOCCO ; CCCCCCCC/C=C\CCCCCCCC(=O)OCCOCCCC(O)(CCCOCCOC(=O)CCCCCCC/C=C\CCCCCCCC)CCOCCN(C)C]

To a solution of compound 4-6 (3.70 g, 10.5 mmol, 1.00 eq) in Py (18.0 mL) was added EDCI (12.1 g, 63.1 mmol, 6.0 eq) at 20 °C, then oleic acid (17.8 g, 63.1 mmol, 17.4 mL, 6.0 eq) was added into the reaction solution at 20 °C dropwise. The resulting solution was stirred at 40 °C for 16 h. TLC (R_{f} of compound 4-6=0.1, and R_{f} of SL31=0.5) indicated none of compound 4-6 was remained, and one new major spot with lager polarity was detected. The reaction solution was concentrated to remove the insoluble. The organic phase was poured into saturated aqueous solution of NH₄Cl (20.0 ml), the organic phase was washed by half-saturated aqueous solution of NaCl (20 ml). The final organic phase was concentrated under reduced pressure to give a residue. The residue was purified by column chromatography (SiO₂, DCM/MeOH=10/1). Compound SL31 (1.00 g, 1.14 mmol, 10.8% yield) was obtained as yellow oil. ¹HNMR: ET60053-55-P1A29 (400 MHz, CDCl₃) δ 0.86-0.92 (m, 6H), 1.23-1.36 (m, 42H), 1.50-1.56 (m, 4H), 1.58-1.67 (m, 8H), 1.74-1.78 (m, 3H), 1.99-2.05 (m, 8H), 2.50-2.58 (m, 2H), 3.43-3.50 (m, 4H), 3.54-3.59 (m, 2H), 3.60-3.68 (m, 6H), 4.19-4.23 (m, 4H), 5.27-5.40 (m, 4H).

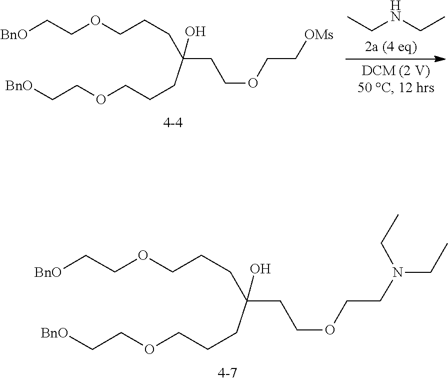

To a solution of compound 2a (4.52 g, 61.8 mmol, 6.36 mL, 4.00 eq) in DCM (9.00 mL) was added compound 4-4 (9.00 g, 15.4 mmol, 1.00 eq). The solution was stirred at 50 °C for 12 h. TLC (Dichloromethane/Methanol=10/1, R_{f} of compound 4-4=0.52, R_{f} of compound 4-7=0.40) showed the reaction was finished. The reaction solution combined and concentrated in vacuum to give crude product. The residue was purified by column chromatography (SiO₂, Petroleum ether/Ethyl acetate=10/1 to 0/1) to give compound 4-7 (7.00 g, 12.5 mmol, 80.9% yield) as yellow oil. ¹H NMR: ET61249-31-P1A (400 MHz, CDCl₃) δ 7.29-7.35 (m, 10H), 4.57 (s, 4H), 3.36-3.66 (m, 10H), 3.46-3.52 (m, 6H), 2.54-2.63 (m, 6H), 1.53-1.75 (m, 11H), 1.02 (t, J=7.2 Hz, 6H).

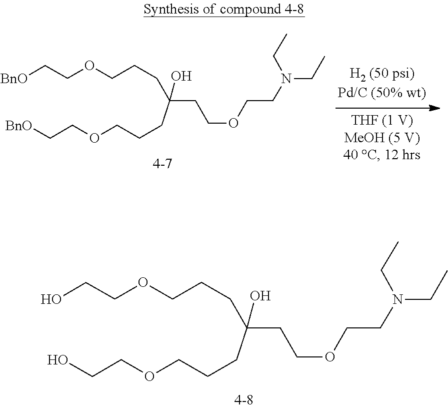 [SMILES: CCN(CC)CCOCCC(O)(CCCOCCOCc1ccccc1)CCCOCCOCc1ccccc1 ; CCN(CC)CCOCCC(O)(CCCOCCO)CCCOCCO]

To a solution of compound 4-7 (7.00 g, 12.5 mmol, 1.00 eq) in MeOH (35.0 mL) and THF (10.0 mL) was added Pd/C (3.50 g, 10% purity) under N₂ atmosphere. The suspension was degassed and purged with H2 for 3 times. The solution was stirred under H2 (50 Psi.) at 40 °C for 12 hrs. LCMS showed that the reaction was finished. The suspension was filtered through a pad of silica gel and filter cake was washed with MeOH (100 mL×3). The combined filtrates were concentrated to dryness to give compound 4-8 (4.50 g, 11.9 mmol, 94.8% yield) as yellow oil. LCMS: ET61249-32-P1A (RT=0.784 min, M+H⁺=381.3). ¹H NMR: ET61249-32-P1A (400 MHz, CDCl₃) δ 3.55-3.72 (m, 4H), 3.52-3.54 (m, 2H), 3.48-3.49 (m, 12H), 2.63-2.63 (m, 6H), 1.55-1.75 (m, 12H), 1.02 (t, J=7.2 Hz, 6H).

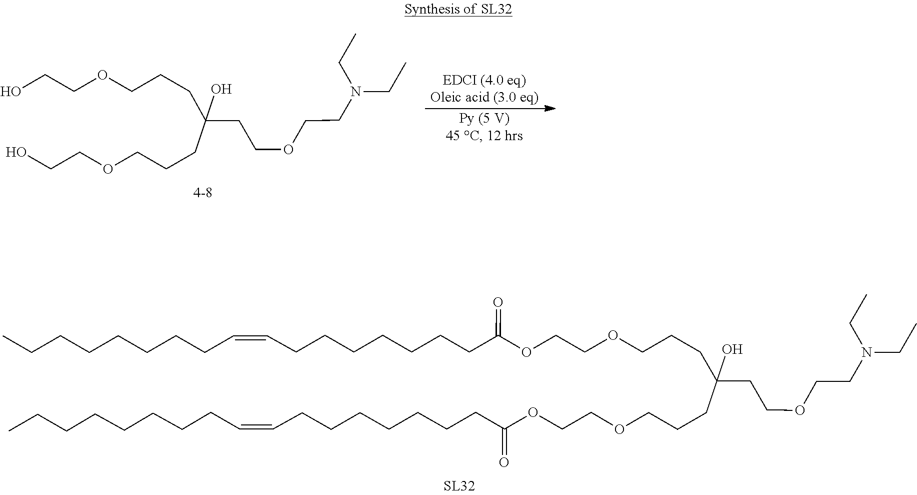 [SMILES: CCN(CC)CCOCCC(O)(CCCOCCO)CCCOCCO ; CCCCCCCC/C=C\CCCCCCCC(=O)OCCOCCCC(O)(CCCOCCOC(=O)CCCCCCC/C=C\CCCCCCCC)CCOCCN(CC)CC]

To a solution of compound 4-8 (3.50 g, 9.22 mmol, 1.00 eq) and (Z)-octadec-9-enoic acid (7.81 g, 27.6 mmol, 7.81 mL, 3.00 eq) in Py (20.0 mL) was added EDCI (7.07 g, 36.9 mmol, 4.00 eq). The solution was stirred at 45 °C for 12 h. LCMS showed that the reaction was finished. The reaction solution was poured into NH₄Cl (sat, 50.0 ml, aq), the solution was extracted with EtOAc (20.0 mL×2), the organic layers were combined and concentrated in vacuum to give crude product. The crude product was purified by reversed-phase HPLC (column: Welch Xtimate C1 100*30 mm*5 um; mobile phase: [water (FA)-ACN]; B %: 40%-80%, 15 min) to give compound 4-7 (1.00 g, 1.10 mmol, 11.9% yield) as light yellow oil. LCMS: ET61249-35-P1A (RT=2.347 min, M+H⁺=908.8). ¹H NMR: ET61249-32-P1A (400 MHz, CDCl₃) δ 5.33-5.37 (m, 4H), 4.21 (t, J=7.2 Hz, 4H), 3.61-3.67 (m, 6H), 3.45-3.53 (m, 6H), 2.35-2.60 (m, 5H), 2.33 (t, J=7.6 Hz, 4H), 1.99-2.20 (m, 8H), 1.63-1.75 (m, 16H), 1.27-1.30 (m, 40H), 1.04 (t, J=7.2 Hz, 6H), 0.88 (t, J=6.8 Hz, 6H).

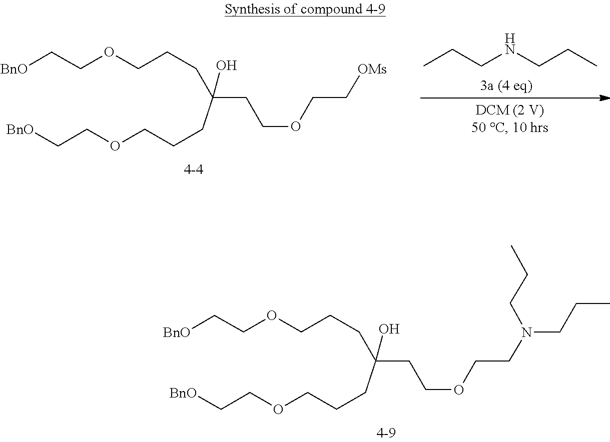

To a solution of compound 4-4 (9.00 g, 15.4 mmol, 1.00 eq) in DCM (18.0 mL) was added compound 3a (6.25 g, 61.8 mmol, 8.52 mL, 4.00 eq). The mixture was stirred at 50 °C for 10 h. LCMS showed compound 4-4 was consumed completely. Several new peaks were shown on LCMS and desired compound was detected. The reaction mixture was partitioned between EtOAc (200 mL) and water (100 mL). The organic layer was dried over Na₂SO₄, filtered and concentrated under reduced pressure to give a residue. The residue was purified by column chromatography (SiO₂, petroleum ether/ethyl acetate=10/1-0/1). Compound 4-9 (5.00 g, 8.51 mmol, 55.1% yield) was obtained as colorless oil. LCMS: ET60350-33-P1A (RT=0.401 min, M+H⁺=588.4). ¹H NMR: ET60350-33-P1A (400 MHz, CDCl₃) δ 0.87 (t, J=7.39 Hz, 6H), 1.42-1.70 (m, 12H), 1.72-1.79 (m, 2H), 2.45 (s, 4H), 2.66 (s, 2H), 3.42-3.55 (m, 6H), 3.55-3.69 (m, 10H), 4.58 (s, 4H), 7.34-7.36 (m, 10H).

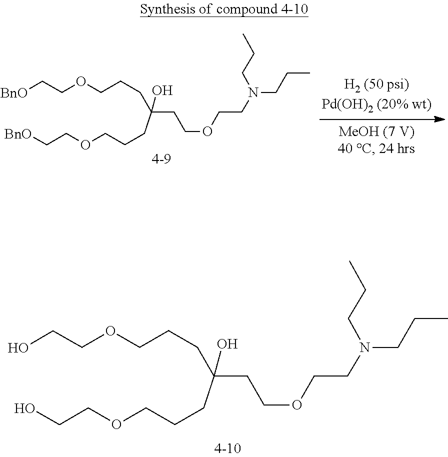 [SMILES: CCCN(CCC)CCOCCC(O)(CCCOCCOCc1ccccc1)CCCOCCOCc1ccccc1 ; CCCN(CCC)CCOCCC(O)(CCCOCCO)CCCOCCO]

To a solution of compound 4-9 (5.00 g, 8.51 mmol, 1.00 eq) in MeOH (35.0 mL) was added Pd(OH)₂ (597 mg, 851 umol, 20% purity, 0.10 eq) under N₂ atmosphere. The suspension was degassed and purged with H2 for 3 times. The solution was stirred under H2 (50.0 Psi) at 40 °C for 24 h. LCMS showed compound 4-9 was consumed completely. Several new peaks were shown on LCMS and desired compound was detected. The suspension was filtered through a pad of celite and the filter cake was washed with MeOH (10.0 mL×3). The combined filtrates were concentrated in vacuum to give a crude product. The crude product was used for next step directly without purification. Compound 4-10 (3.00 g, 7.36 mmol, 86.5% yield) was obtained as colorless oil. LCMS: ET60350-34-P1A (RT=1.281 min, M+H⁺=406.4). ¹H NMR: ET60350-34-P1A (400 MHz, CDCl₃) δ 0.81-0.91 (m, 6H), 1.41-1.48 (m, 4H), 1.52-1.68 (m, 9H), 1.72-1.81 (m, 3H), 2.34-2.46 (m, 5H), 2.57-2.68 (m, 2H), 3.46-3.51 (m, 6H), 3.53-3.56 (m, 4H), 3.63-3.67 (m, 2H), 3.70-3.74 (m, 4H).

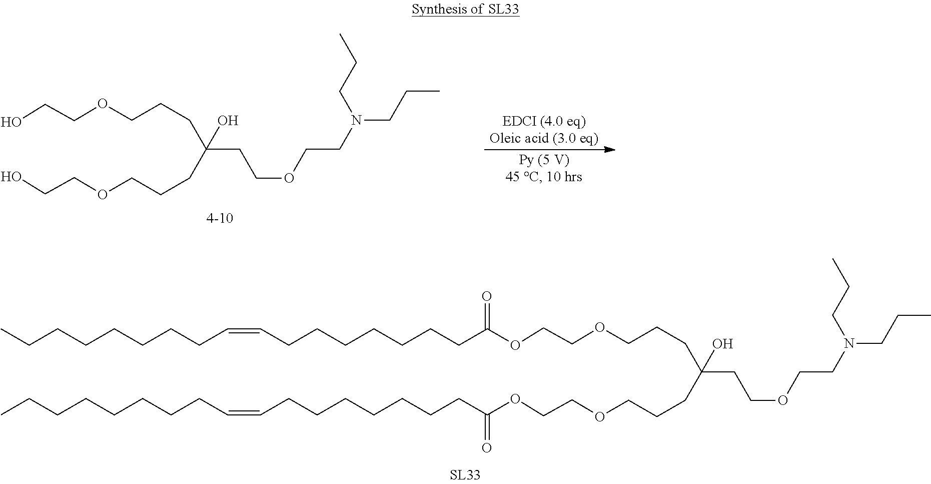 [SMILES: CCCN(CCC)CCOCCC(O)(CCCOCCO)CCCOCCO ; CCCCCCCC/C=C\CCCCCCCC(=O)OCCOCCCC(O)(CCCOCCOC(=O)CCCCCCC/C=C\CCCCCCCC)CCOCCN(CCC)CCC]

To a solution of compound 4-10 (3.00 g, 7.36 mmol, 1.00 eq) in Py (15.0 mL) was added Oleic acid (6.24 g, 22.1 mmol, 6.24 mL, 3.00 eq) and EDCI (5.64 g, 29.4 mmol, 4.00 eq). The mixture was stirred at 45 °C for 10 h. LCMS showed compound 4-9 was consumed completely. Several new peaks were shown on LCMS and desired compound was detected. The reaction mixture was partitioned between EtOAc (200 mL) and water (100 mL). The organic layer was dried over Na₂SO₄, filtered and concentrated under reduced pressure to give a residue. The crude product was purified by reversed-phase HPLC (column: Welch Xtimate C1 100*30 mm*5 um; mobile phase: [water (FA)-ACN]; B %: 30%-65%, 10 min). Compound SL33 (1.00 g, 1.07 mmol, 14.5% yield) was obtained as yellow oil. LCMS: ET60350-35-P1A (RT=2.824 min, M+H⁺=937.0). ¹H NMR: ET60350-35-P1A8 (400 MHz, CDCl₃) δ 0.75-1.04 (m, 12H), 1.24-1.35 (m, 40H), 1.45-1.68 (m, 16H), 1.73-1.78 (m, 2H), 1.99-2.03 (m, 8H), 2.31-2.37 (m, 4H), 2.38-2.51 (m, 3H), 2.56-2.78 (m, 2H), 3.42-3.58 (m, 6H), 3.59-3.68 (m, 6H), 4.16-4.25 (m, 4H), 5.28-5.40 (m, 4H).

### Example 2: Formulation of Ionizable Cationic Lipids into LNPs with a mRNA

This is a general description of how each ionizable cationic lipid was used to formulate an samRNA LNP. The specifics for each lipid are listed in Table 1. The ionizable cationic lipid (see Table 1), 1,2-distearoyl-sn-glycero-3-phosphocholine (DSPC), cholesterol, and 1,2-dimyristoyl-rac-glycero-3-methylpolyoxyethylene (DMG-PEG) were combined in a 40:10:48:2 molar ratio in ethanol at a concentration of 3.2 mM (for 0.5 mg samRNA scale) or 6.4 mM (for 1.5 mg samRNA scale). A solution of samRNA expressing the antigen of interest in 50 mM citrate buffer at pH 6 with 10 mM Tris (2-carboxyethyl) phosphine (TCEP) was prepared. When mixing at an aqueous to ethanol flow rate ratio (FRR) of 2:1, the RNA concentration was 0.025 mg/mL (for 0.5 mg samRNA scale) or 0.050 mg/mL (for 1.5 mg samRNA scale). When mixing at an aqueous to ethanol flow rate ratio (FRR) of 3:1, the RNA concentration was 0.017 mg/mL (for 0.5 mg samRNA scale). The lipid solution in ethanol was then rapidly mixed with the samRNA solution using a staggered herringbone micromixer chip using a Nanoassemblr benchtop instrument (Precision Nanosystems) at a total flow rate (TFR) of 12 mL/min and a flow rate ratio (FRR) of either 2:1 or 3:1 aqueous buffer to ethanol. This mixing ratio results in a 8:1 molar ratio of ionizable cationic lipid (see Table 1) to samRNA phosphate groups and a total lipid to samRNA mass ratio of 37:1. The resulting mixed solution was then diluted 10-fold into 50 mM citrate buffer at pH 6 with 10 mM TCEP and subjected to tangential flow filtration (TFF) using a 300 k molecular weight cut-off membrane (mPES) until concentrated to the original volume. Subsequently, the citrate buffer was replaced with a buffer containing 20 mM Tris buffer at pH 7.5, 80 mM sodium chloride, and 3% sucrose using diafiltration with 10 diavolumes. The LNP solution was concentrated to a volume between 4-10 mL, filtered using a 0.2 micron PES syringe filter, aliquoted into vials, and frozen at 1 °C/min using a Corning® CoolCell® LX Cell Freezing Container until the samples reached −80 °C. Samples were stored at −80 °C and thawed on wet ice before analysis or use. The total RNA concentration, the percentage of input RNA recovered (% recovery), and the encapsulation efficiency (% EE) were determined using a Ribogreen assay, which is described elsewhere. The Z-avg diameter (nm) and polydispersity index (PDI) were measured using dynamic light scattering (Malvern Zetasizer) following a 1:100 dilution in phosphate buffered saline (PBS).

| | | | Input | | | | | | Z-average | |
|---|---|---|---|---|---|---|---|---|---|---|
| | Ionizable | Antigens | RNA | Flow-rate ratio | Citrate | Encapsulation | Total RNA | RNA | diameter | Polydispersity |
| | Cationic | expressed - | scale | (FRR, | buffer | efficiency | concentration | recovery | by DLS | index (PDI) |
| LNP# | Lipid | samRNA | (mg) | Aqueous:Ethanol) | pH | (EE, %) | (μg/mL) | (%) | (nm) | by DLS |
| 1 | SL26 | H5, N1 | 0.5 | 3:1 | 6 | 73.8 | 38.4 | 48.3 | 210.2 | 0.433 |
| 2 | SL27 | H5, N1 | 0.5 | 3:1 | 6 | 79.1 | 32.8 | 37.5 | 401.7 | 0.342 |
| 3 | SL28 | H5, N1 | 0.5 | 3:1 | 6 | 97.8 | 11.9 | 15.9 | 180.9 | 0.251 |
| 4 | SL29 | H5, N1 | 0.5 | 3:1 | 6 | 93.9 | 7.3 | 6.7 | 295 | 0.171 |
| 5 | SL30* | H5, N1 | 0.5 | 3:1 | 6 | N/A | N/A | N/A | N/A | N/A |
| 6 | SL31 | H5, N1 | 0.5 | 3:1 | 6 | 99.7 | 41 | 65.3 | 117.5 | 0.175 |
| 7 | SL32 | H5, N1 | 0.5 | 3:1 | 6 | 99.7 | 45.8 | 66.3 | 110.2 | 0.181 |
| 8 | SL33 | H5, N1 | 0.5 | 3:1 | 6 | 68.4 | 46.5 | 68.6 | 113.9 | 0.143 |

*The formulation using SL30 failed due to a clogging problem likely caused by large particle size.

### Biophysical Characterisation of Lipid Nanoparticles

The messenger RNA-containing LNP composition was characterized using analytical methods to determine the loading of messenger RNA, the percentage of messenger RNA that is encapsulated, and the size of the particles. The total amount of messenger RNA contained in the sample and the percentage of that messenger RNA that is encapsulated was determined using a fluorescence assay employing Ribogreen, a dye that becomes more emissive upon binding messenger RNA. The total amount of messenger RNA was determined by disrupting the LNP with 1 wt % Triton-X 100 to expose the encapsulated messenger RNA, adding the dye, and comparing the emission intensity against a standard curve prepared using ribosomal RNA. The amount of unencapsulated messenger RNA was measured in a similar manner with the detergent disruption of the LNP is omitted. With the total amount of messenger RNA known and the amount of unencapsulated messenger RNA known, the percent encapsulated messenger RNA was calculated thus:

Percent Encapsulation (%)=((RNA_{TOTAL}−RNA_{UNENCAPSULATED})/RNA_{TOTAL})×100
where RNA_{TOTAL} and RNA_{UNENCAPSULATED} are, respectively, the concentrations of total messenger RNA and unencapsulated messenger RNA. The total messenger RNA content varied based on formulation, but generally fell in the range of 0.030-0.200 mg/mL. The size of LNP was measured using dynamic light scattering of a sample diluted 1:100 in PBS buffer.

### pKa Protocol

In a black 96-well plate, solutions of samRNA-LNP (final assay concentration 2 μg/mL total RNA) in a series of buffers ranging from pH 4 to 9.5 will be prepared. Buffers between pH 4 to 7.6 will be prepared from disodium phosphate and citric acid. Buffers from 7.8 to 9.5 will be prepared by titrating tris buffer with 10 N sodium hydroxide. To each well will be added 6-(p-Toluidino)-2-naphthalenesulfonic acid sodium salt (TNS) in water to a final assay concentration of 6 μM. The fluorescence will be read on a plate reader at 25 °C with an excitation setting of 321 nm and an emission setting of 445 nm. The intensity values will be plotted as a function of pH using GraphPad Prism and be fit with a sigmoidal dose response curve. The apparent pKa will be determined as the EC50 of this curve where half of the ionizable amines are expected to be protonated.

### Characterization of the In Vitro Potency of LNPs

The ability of the samRNA-LNPs to transfect cultured cells will be characterized using an in vitro assay based on the percentage of cells expressing the antigen of interest. Specifically for each LNP, 1 million BHK-21 cells will be co-incubated with samRNA-LNP of varying concentration in 0.3 mL of media for 17-19 hours at 37 °C with 5% CO₂. Subsequently, the cells will be treated with TrypLE (Gibco) to detach from the dish to form a single-cell suspension, fixed and permeabilized (BD Cytofix/Perm kit), and then stained with fluorophore-labeled antigen-specific antibodies against H5 and N₁. The percentage of double antigen positive cells will be quantified using a BD Accuri flow cytometer.

### Characterization of the In Vivo Potency of LNPs

The ability of mRNA-LNPs to act as a vaccine will be evaluated by measuring the antibody- and cell-based immune response following a prime-boost vaccination schedule. A priming vaccination will be given to Balb/c mice on Day 0 via intramuscular injection (i.m.) and followed 21 days later with a boosting vaccination. After an additional 21 days (Day 42 of the experiment), the mice will be sacrificed and serum and splenocytes will be collected for further analysis.

Serum will be analysed for vaccine-specific antibody response using an IgG ELISA, a pseudovirus microneutralization assay, a hemagglutination inhibition (HAI) assay, and an enzyme-linked lectin assay (ELLA). The results will be compared to an adjuvanted inactivated virus vaccine (aH5N1) tested in the same experiment using a one-way ANOVA statistical test.

Splenocytes from the experiment described above will be analysed for antigen-specific cytokine production using intracellular cytokine staining by flow cytometry following in vitro peptide stimulation. Splenocytes will be pooled (n=5/group) and stimulated ex vivo in the absence or presence of H5 or N1 peptides in duplicate. The mean level of response, measured by interferon gamma, interleukin-2, and/or tumor necrosis factor alpha production, will be displayed and error bars display the measurement precision.

Taken together, these in vivo results are expected to demonstrate that LNPs prepared using the lipids SL26-SL33 are immunogenic and effective as an influenza vaccine in this preclinical model.

### Characterization of the In Vivo Transfection Efficiency of LNPs

The in vivo potency of mRNA-LNPs will be evaluated using samRNA expressing the reporter protein firefly luciferase in order to quantify the location, relative amount, and the duration of protein expression. LNPs formulated with a mRNA expressing luciferase will be injected into mice, for example intramuscularly in the hind leg. At defined time points, such as daily, the mice will be administered luciferin and the bioluminescence will be imaged and quantified.

### Characterization of Genotoxic Potential, Tolerability, Biodistribution, and Biodegradability

A standard test battery for prediction of genotoxic potential (damage of DNA) will be used, as no single test can detect all genotoxic mechanisms leading to tumorigenicity. As an example, when a positive result is seen in in vitro mammalian cell assay, clearly negative results in two in vivo assays, in appropriate tissues and demonstrated sufficient test substance exposure, will be considered evidence for lack of genotoxic potential in vivo. Relevant guidelines, such as S2(R1) Genotoxicity Testing and Data Interpretation for Pharmaceuticals Intended for Human Use”, will be followed.
Test for gene mutation in bacteria.
In vitro cytogenetic test for chromosomal damage (metaphase chromosome aberration or micronucleus test) or mouse lymphoma Tk gene mutation assay.
In vivo genotoxicity test for chromosomal damage using rodent hematopoietic cells either for micronuclei or chromosomal aberrations in metaphase cells.

### In Silico Screening of Novel Lipids for Toxicity

Compounds of Formula I will be screened for potential toxicity and mutagenicity using commercially available computational toxicology assessment products and/or services to satisfy International “Council for Harmonisation of Technical Requirements for Pharmaceuticals for Human Use (ICH) M7(R1) Assessment and Control of DNA Reactive (Mutagenic) Impurities in Pharmaceuticals To Limit Potential Carcinogenic Risk”. For example, two complementing Quantitative (Q)SAR methods, expert rule-based Derek Nexus and statistical-based Sarah Nexus (or Leadscope), will be used to predict outcome of bacterial mutagenicity. Classification will be based on Class 1 to 5 with respect to mutagenic and carcinogenic potential from known mutagenic carcinogen (Class 1) to lack of mutagenicity or carcinogenicity (Class 5). The outcome of computer-based analysis will be reviewed for relevance of positive, negative, conflicting, or inconclusive prediction and rationale for conclusion provided.

As most structural alerts are based on bacterial mutagenicity, compounds with structural alert can be detected in standard test battery. In addition, some chemical classes are more easily detected in mammalian cell chromosome damage assays than bacterial mutation assays. A negative result from a compound with structural alert in either test battery should be considered not genotoxic.

### In Vitro Screening for Genotoxicity

Compounds of Formula I will be assessed first for mutagenicity in bacterial reverse gene mutation test (Ames), which detects relevant genetic changes and most genotoxic rodent and human carcinogens and then for genotoxicity in vitro micronucleus (MN) assay using mammalian cells:

Ames assay will follow Organization for Economic Co-operation and Development (OECD) Guidelines for the Testing of Chemicals No. 471 Bacterial Reverse Mutation Test and S2(R1) Genotoxicity Testing and Data Interpretation for Pharmaceuticals Intended for Human Use to assess mutagenic potential. At least five strains of bacteria Salmonella typhimurium TA98, TA100, TA1537, TA1535 and TA102 or E. coli WP2 will be exposed to test substance in the presence and absence of metabolic activation system rat liver metabolizing system (S—9).

In vitro mammalian cell micronucleus genotoxicity test will follow OECD Guidelines for the Testing of Chemicals No. 487. This test should detect MN in the cytoplasm of interphase cells and chromosome damaging potential (aneugens and clastrogens). Sufficiently validated and appropriate are mouse lymphoma L5178Y cell Tk (thymidine kinase) gene mutation assay (MLA) and human lymphocyte micronucleus assay (HLM) treated in the absence and presence of rat liver metabolising system (S—9).

### In Vivo Screening for Genotoxicity

Compounds of Formula I will be assessed in vivo in either acute or repeat-dose rat study, as some agents are mutagenic in vivo but not in vitro. The choice of analysis will be micronuclei in erythrocytes (in blood or bone marrow) or chromosome aberrations in metaphase cells in bone marrow.

### Biodistribution and Biodegradability Assessment

Ex vivo biodegradability assessment: Compounds of Formula I will be screened for enzymatic biodegradation using enzyme-containing solutions prepared from relevant species, such as human, mouse, and rat. Lipids will be screened both in their neat form (unformulated) and when incorporated into LNPs that do or do not contain RNA. For example, a novel ionizable cationic lipid will be diluted at an approximate concentration of 1.0 to 0.001 mg/mL in an aqueous solution of human or rat liver microsomes and incubated at temperatures in the range 25-37 °C for durations from 0.1-24 hours. Subsequently, the amount of intact lipid remaining will be measured using liquid chromatography (LC) with evaporative light scattering (ELS) or mass spectrometry (MS) detection and compared to control samples that were not treated with enzymes or were incubated at 2-8 °C to inhibit enzymatic activity. Furthermore, the appearance of new peaks in the chromatograph, which presumably represent degradation products, will be investigated using MS to confirm identity. Without wishing to be bound by theory, the expectation is that degradation may occur via ester hydrolysis and that the lipid's structure will influence the rate of ester hydrolysis.

In vivo biodistribution, pharmacokinetics, and biodegradability assessment: Assessment of biodistribution and pharmacokinetics [Absorption, Distribution, Metabolism, and Excretion) (ADME))] will be performed in rat, which will receive the novel lipids (in their neat form and/or incorporated into LNPs that do or do not contain RNA) intramuscularly or intravenously.

Assessment of biodistribution will be based on the principles of ICH-M3(R2) and World Health Organization (WHO) guidelines on nonclinical evaluation of vaccines, WHO Technical Report Series No. 927, Annex 1. Blood samples will be collected multiple times for lipid, mRNA and immunogenicity analysis. Tissues will be collected at predefined days and lipid analyses performed with qualified liquid chromatography-mass spectrometry method (LS-MS/MS). If needed, RT qPCR analyses of the nucleic acid payload will be performed.

Lipid pharmacokinetics in plasma will be evaluated with qualified LS-MS/MS.

In vitro biodegradability assessment: Compounds of Formula I will be screened by testing the stability of the test compound in the presence of cryopreserved hepatocytes. For example, a suspension of cryopreserved hepatocytes (final cell density 0.5×106 viable cells/mL in Williams E media supplemented with 2 mM L glutamine and 25 mM HEPES) are pre-incubated at 37 °C prior to the addition of test compound (final substrate concentration 1 μM; final DMSO concentration 0.25%) to initiate the reaction. Two control compounds are included with each species. Each compound is incubated for 0, 5, 10, 20, 40 and 60 min at 37 °C. The reactions are stopped by transferring incubate into acetonitrile at the appropriate time points, in a 1:3 ratio. The termination plates are centrifuged at 3,000 rpm for 30 min at 4 °C to precipitate the protein. Following protein precipitation, the sample supernatants are combined in cassettes of up to four compounds, internal standard is added and samples analysed using Cyprotex generic LC MS/MS conditions.

### In Vivo Tolerability Assessment

The tolerability of Compounds of Formula I will be evaluated in appropriate species, such as mice or rats. For example, groups of Sprague-Dawley rats will be administered novel-lipid-containing RNA LNP vaccines at doses ranging from 0.1 to 30 pg total RNA per animal via intramuscular injection. Each animal will be given between 1-3 injections and the effect of treatment on weight loss, food intake, and body temperature will be measured. In addition, the injection site will be monitored using the Draize dermal irritation scoring system. In addition, serum will be collected to evaluate the vaccine immune response using ELISA and functional antibody assays. In addition, the complete blood count, blood chemistry, and blood coagulation will be assessed at one or more timepoint during the study. At the end of the study during necropsy select organs may be macroscopically assessed and weighed. Novel lipids associated with RNA LNP vaccines that maintain high immunogenicity while causing the least undesired responses, such as weight loss, fever, or injection site reactogenicity, will be considered more promising candidates over lipids with a less attractive tolerability profile.

### Toxicity Assessment

Repeat-dose general toxicity study: Toxicity of the candidate Compounds of Formula I will be assessed in a pharmacological relevant nonclinical species (e.g. rat). Novel-lipid-containing RNA/LNP vaccines will be administered in Sprague-Dawley rats once every three weeks (total of 3 doses) via intramuscular injection. The reversibility or persistence of any effects will be assessed after a 3-week recovery phase.

Assessment of toxicity will be based on mortality, clinical observations, body weights, food consumption, ophthalmic observations, dose site (dermal) observations, body temperatures, and clinical and anatomic pathology, micronucleus analysis. Blood samples will be collected for immunogenicity analysis.

### Itemized Listing of Embodiments

1. A compound of Formula I:

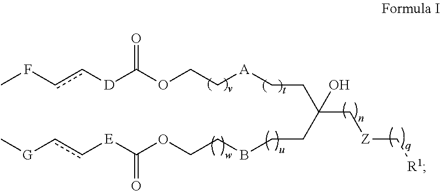

or a pharmaceutically acceptable salt, prodrug or stereoisomer thereof, wherein:
   R¹ is selected from the group consisting of Formulae I-A to I-I:

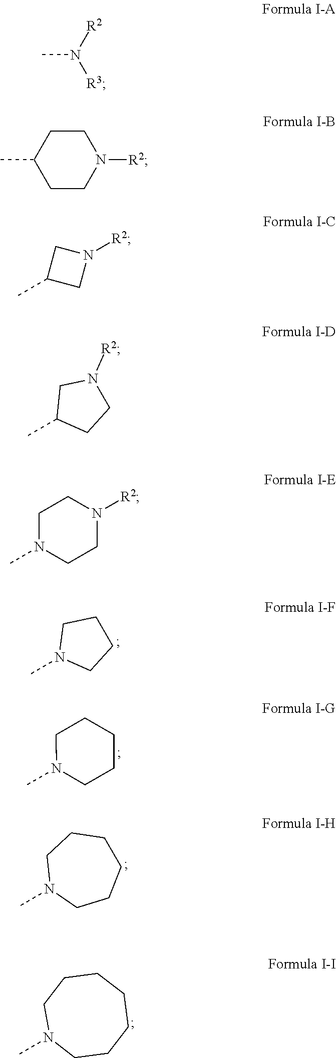

Z, A, and B are each independently selected from the group consisting of —C(H)₂—, —N(H)—, —O—, and —S—; wherein at least one of Z, A, and B is —N(H)—, —O—, or —S—;
n and q are each independently an integer from 0 to 8;
t, u, v, and w are each independently an integer from 0 to 10;
D, E, F, and G are each independently a linear or branched C₁₋₁₆-alkyl;
[symbol] represents a single or double bond; and
R² and R³ are each independently selected from the group consisting of C₁₋₈-alkyl and C₁₋₈-alkanol.
2. The compound of item 1, wherein R² is C₁₋₈-alkyl.
3. The compound of item 1 or item 2, wherein R² is C₁₋₃-alkyl.
4. The compound of any one of items 1 to 3, wherein, if present, R³ is C₁₋₈-alkyl.
5. The compound of any one of items 1 to 4, wherein, if present, R³ is C₁₋₃-alkyl.
6. The compound of any one of items 1 to 5, wherein R¹ is Formula I-A.
7. The compound of any one of items 1 to 6, wherein R¹ is Formula I-A selected from the group consisting of:

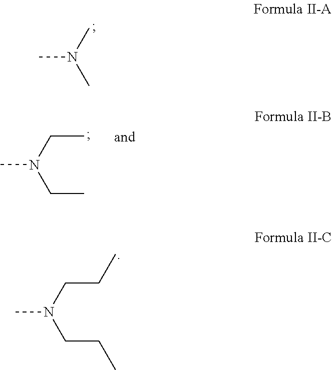

8. The compound of any one of items 1 to 5, wherein R¹ is Formula I-B.
9. The compound of any one of items 1 to 5 and 8, wherein R¹ is Formula I-B selected from the group consisting of:

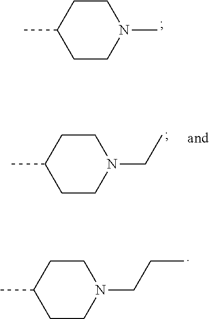

10. The compound of any one of items 1 to 9, wherein Z is selected from the group consisting of —N(H)—, —O—, or —S—.
11. The compound of any one of items 1 to 10, wherein Z is —O—.
12. The compound of any one of items 1 to 11, wherein n and q are each independently an integer from 0 to 4.
13. The compound ofany one of items 1 to 12, wherein n is 2.
14. The compound of any one of items 1 to 13, wherein q is 2.
15. The compound of any one of items 1 to 14, wherein n is 2 and q is 2.
16. The compound of any one of items 1 to 15, wherein A is selected from the group consisting of —N(H)—, —O—, and —S—.
17. The compound of any one of items 1 to 16, wherein A is —O—.
18. The compound of any one of items 1 to 17, wherein B is —N(H)—, —O—, and —S—.
19. The compound of any one of items 1 to 18, wherein B is —O—.
20. The compound of any one of items 1 to 19, wherein D, E, F, and G are each independently a linear C₁₋₁₆-alkyl.
21. The compound of any one of items 1 to 20, wherein D, E, F, and G are each independently a linear C₁₋₈-alkyl.
22. The compound of any one of items 1 to 19, wherein at least one of D, E, F, and G is a branched C₁₋₁₆-alkyl.
23. The compound of any one of items 1 to 19 and 22, wherein at least one of D, E, F, and G is a branched C₁₋₈-alkyl.
24. The compound of any one of items 1 to 23, wherein [symbol] is a double bond.
25. The compound of any one of items 1 to 24, wherein the compound of Formula I is a compound of Formula III-A:

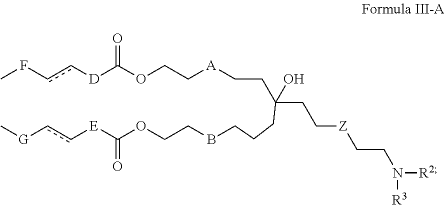

wherein R², R³, Z, A, B, D, E, F, and G are as defined herein.
26. The compound of any one of items 1 to 25, wherein the compound of Formula I is a compound of Formula IV-A:

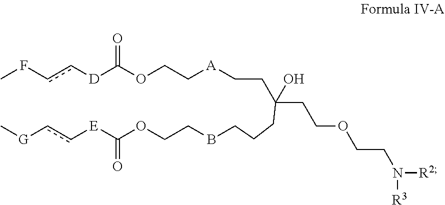

wherein R², R³, A, B, D, E, F, and G are as defined herein
27. The compound of any one of items 1 to 26, wherein the compound of Formula I is a compound of Formula V-A:

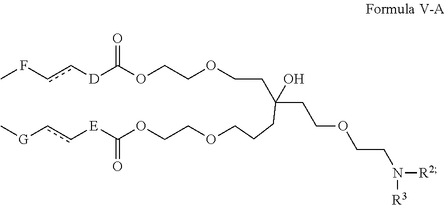

wherein R², R³, D, E, F, and G are as defined herein.
28. The compound of any one of items 1 to 27, wherein the compound of Formula I is selected from the group consisting of:

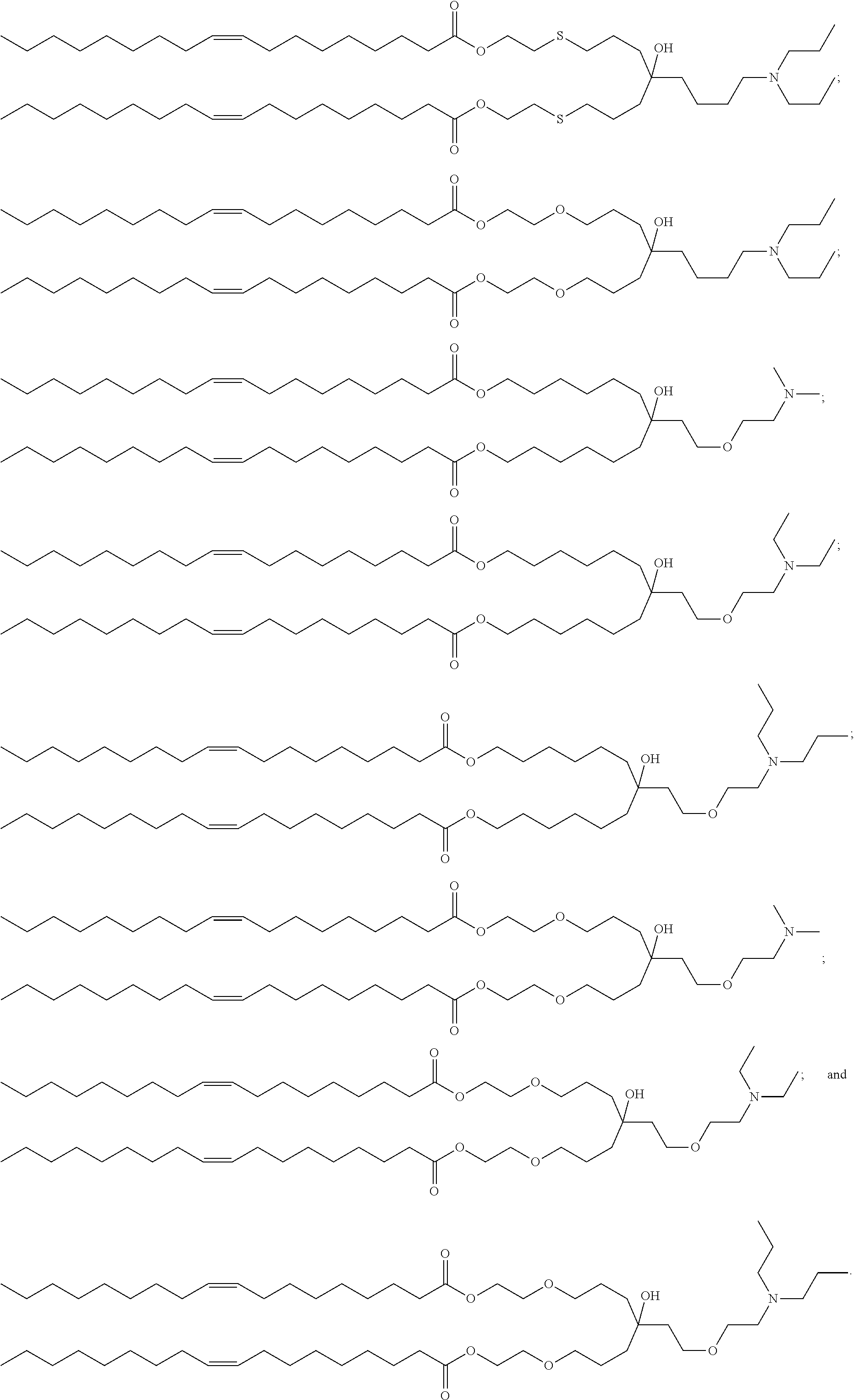

29. A lipid nanoparticle (LNP) comprising a lipid component comprising a compound of any one of items 1 to 28.
30. The lipid nanoparticle of item 29, wherein the lipid component further comprises one or more of a neutral lipid, a structural lipid, and a PEGylated lipid.
31. The lipid nanoparticle of item 30, wherein the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.
32. The lipid nanoparticle of item 30, wherein the structural lipid is selected from the group consisting of cholesterol, fecosterol, sitosterol, campesterol, stigmasterol, brassicasterol, ergosterol, tomatidine, tomatine, ursolic acid and alpha-tocopherol.
33. The lipid nanoparticle of item 30, wherein the PEGylated lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, optionally PEG-c-DOMG, PEG-DMG, PEG-DLPE, PEG-DMPE, PEG-DPPC, and PEG-DSPE.
34. The lipid nanoparticle of item 30, wherein the lipid component comprises: about mol % to about 60 mol % of a compound of any one of claim 1 to claim 6; about 2 mol % to about 25 mol % neutral lipid; about 18.5 mol % to about 60 mol % structural lipid; and about 0.2 mol % to about 10 mol % of PEGylated lipid.
35. The lipid nanoparticle of any one of items 29 to 34, wherein the lipid nanoparticle further comprises a polynucleotide.
36. The lipid nanoparticle of item 35, wherein the polynucleotide is selected from the group consisting of: a messenger RNA (mRNA), a self-amplifying mRNA (sa-mRNA), a small interfering RNA (siRNA), a microRNA (miRNA), miRNA inhibitors (antagomirs/antimirs), messenger-RNA-interfering complementary RNA (micRNA), short hairpin RNA (shRNA), multivalent RNA, dicer substrate RNA, an antisense oligonucleotide, plasmid DNA, DNA, and complementary DNA (cDNA).
37. The lipid nanoparticle of item 35 or item 36, wherein the polynucleotide is a ribonucleic acid (RNA).
38. The lipid nanoparticle of any one of items 35 to 37, wherein the polynucleotide is a conventional or self-amplifying mRNA.
39. The lipid nanoparticle of any one of items 29 to 38, wherein the lipid nanoparticle has a diameter of from about 30 nm to about 160 nm.
40. A pharmaceutical composition comprising a plurality of lipid nanoparticles of any one of items 29 to 39, and a pharmaceutically acceptable carrier.
41. A method of delivering a polynucleotide to a mammalian cell, including administering the lipid nanoparticle of any one of items 29 to 39, or the pharmaceutical composition of item 40, to a subject to thereby contact the cell with the lipid nanoparticle or pharmaceutical composition and deliver the polynucleotide to the cell.
42. The method of item 41 wherein the cell is a cell of a human subject.
43. A method of producing a polypeptide of interest in a mammalian cell, including the step of contacting the cell with a lipid nanoparticle of any one of items 29 to 39, or the pharmaceutical composition of item 40, wherein the lipid nanoparticle comprises a conventional mRNA or a self-amplifying mRNA encoding the polypeptide.
44. A method of treating a disease, disorder or condition in a subject in need of such treatment, comprising administering a lipid nanoparticle of any one of items 29 to 39, or the pharmaceutical composition of item 40, to the subject to thereby treat the disease, disorder or condition.
45. The method of item 44, wherein the disease, disorder or condition is selected from the group consisting of a rare disease, an infectious disease, cancer, a proliferative disease, a genetic disease, an autoimmune disease, diabetes, a neurodegenerative disease, a cardiovascular disease, a reno-vascular disease and a metabolic disease.
46. A vaccine comprising a lipid nanoparticle of any one of items 29 to 39, or the pharmaceutical composition of item 40, and a mRNA encoding a polypeptide.
47. The vaccine of item 46, wherein the vaccine is selected from a tumor vaccine, an influenza vaccine, and a SARS, including a SARS-CoV-2, vaccine.

1. A compound of Formula I: 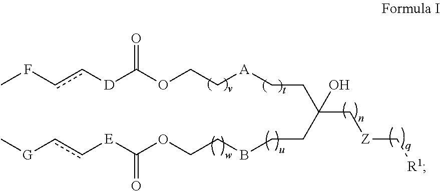
or a pharmaceutically acceptable salt, prodrug or stereoisomer thereof, wherein:
R¹ is selected from the group consisting of Formulae I-A to I-I: 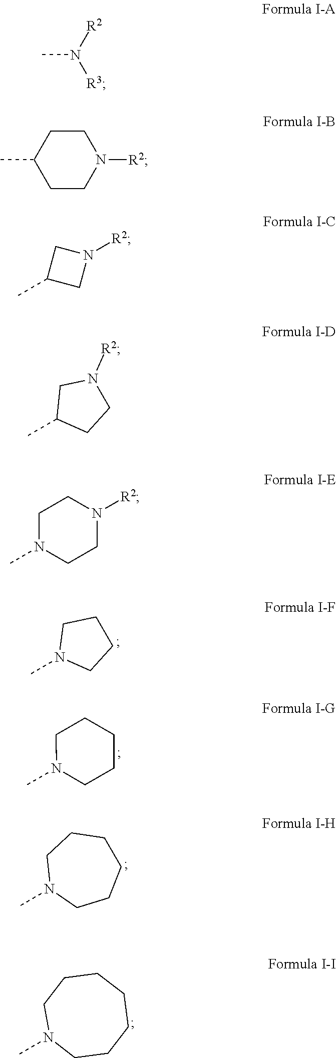
Z, A, and B are each independently selected from the group consisting of —C(H)₂—, —N(H)—, —O—, and —S—; wherein at least one of Z, A, and B is —N(H)—, —O—, or —S—;
n and q are each independently an integer from 0 to 8;
t, u, v, and w are each independently an integer from 0 to 10;
D, E, F, and G are each independently a linear or branched C₁₋₁₆-alkyl; [symbol]
represents a single or double bond; and
R² and R³ are each independently selected from the group consisting of C₁₋₈-alkyl and C₁₋₈-alkanol.

2. The compound of claim 1, wherein R² is C₁₋₈-alkyl.

3. The compound of claim 1, wherein R² is C₁₋₃-alkyl.

4. The compound of claim 1, wherein, if present, R³ is C₁₋₈-alkyl.

5. The compound of claim 1, wherein, if present, R³ is C₁₋₃-alkyl.

6. The compound of claim 1, wherein R¹ is Formula I-A.

7. The compound of claim 1, wherein R¹ is Formula I-A selected from the group consisting of:

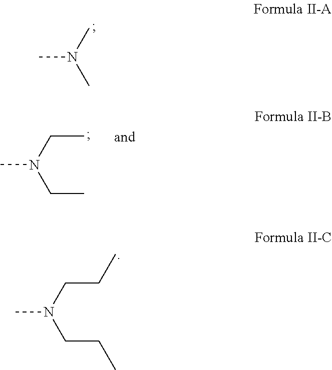

8. The compound of claim 1, wherein R¹ is Formula I-B.

9. The compound of claim 1, wherein R¹ is Formula I-B selected from the group consisting of:

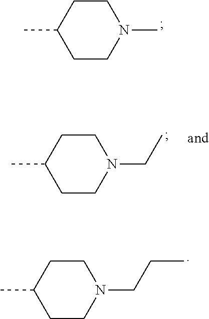

10. The compound of claim 1, wherein Z is selected from the group consisting of —N(H)—, —O—, or —S—.

11. The compound of claim 1, wherein Z is —O—.

12. The compound of claim 1, wherein n and q are each independently an integer from 0 to 4.

13. The compound of claim 1, wherein n is 2.

14. The compound of claim 1, wherein q is 2.

15. The compound of claim 1, wherein n is 2 and q is 2.

16. The compound of claim 1, wherein A is selected from the group consisting of —N(H)—, —O—, and —S—.

17. The compound of claim 1, wherein A is —O—.

18. The compound of claim 1, wherein B is —N(H)—, —O—, and —S—.

19. The compound of any one of claims 1 to 18, wherein B is —O—.

20. The compound of claim 1, wherein D, E, F, and G are each independently a linear C₁₋₁₆-alkyl.

21. The compound of claim 1, wherein D, E, F, and G are each independently a linear C₁₋₈-alkyl.

22. The compound of claim 1, wherein at least one of D, E, F, and G is a branched C₁₋₁₆-alkyl.

23. The compound of claim 1, wherein at least one of D, E, F, and G is a branched C₁₋₈-alkyl.

24. The compound of claim 1, wherein [symbol] is a double bond.

25. The compound of claim 1, wherein the compound of Formula I is a compound of Formula III-A: 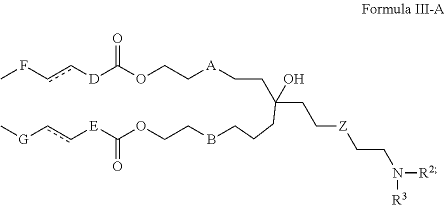
wherein R², R³, Z, A, B, D, E, F, and G are as defined herein.

26. The compound of claim 1, wherein the compound of Formula I is a compound of Formula IV-A: 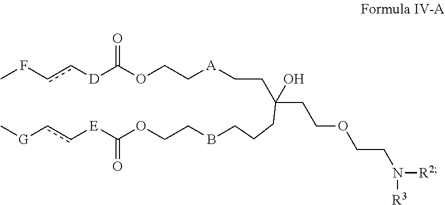
wherein R², R³, A, B, D, E, F, and G are as defined herein

27. The compound of claim 1, wherein the compound of Formula I is a compound of Formula V-A: 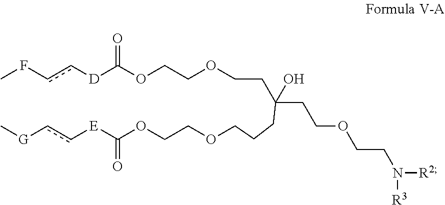
wherein R², R³, D, E, F, and G are as defined herein.

28. The compound of claim 1, wherein the compound of Formula I is selected from the group consisting of:

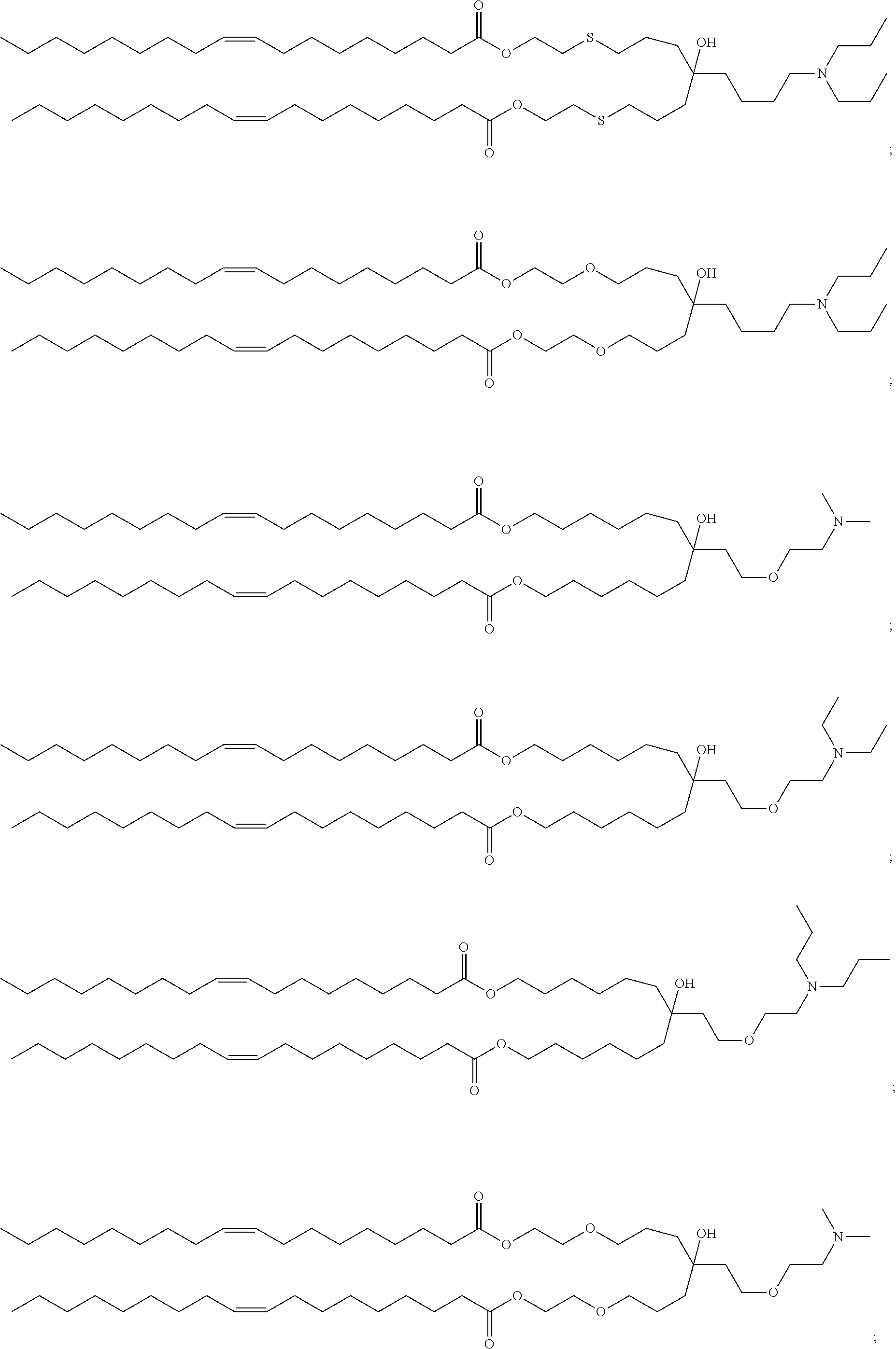

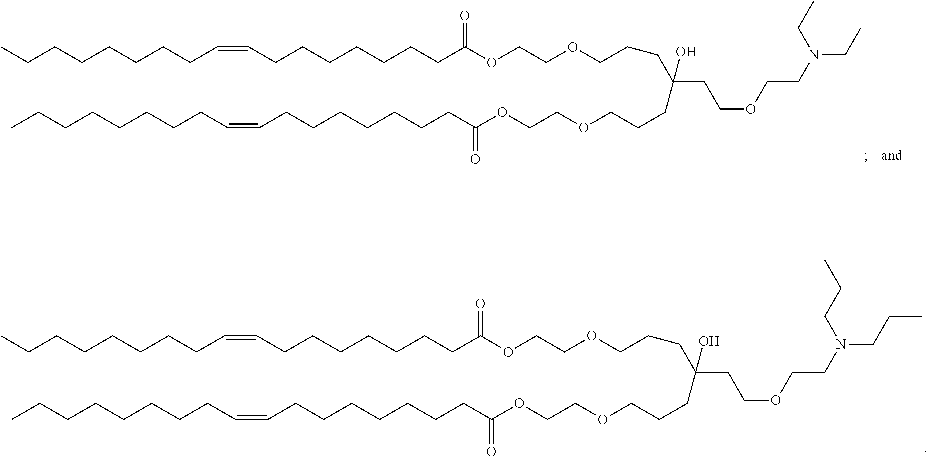

29. A lipid nanoparticle (LNP) comprising a lipid component comprising a compound of claim 1.

30. The lipid nanoparticle of claim 29, wherein the lipid component further comprises one or more of a neutral lipid, a structural lipid, and a PEGylated lipid.

31. The lipid nanoparticle of claim 30, wherein the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

32. The lipid nanoparticle of claim 30, wherein the structural lipid is selected from the group consisting of cholesterol, fecosterol, sitosterol, campesterol, stigmasterol, brassicasterol, ergosterol, tomatidine, tomatine, ursolic acid and alpha-tocopherol.

33. The lipid nanoparticle of claim 30, wherein the PEGylated lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, optionally PEG-c-DOMG, PEG-DMG, PEG-DLPE, PEG-DMPE, PEG-DPPC, and PEG-DSPE.

34. The lipid nanoparticle of claim 30, wherein the lipid component comprises: about mol % to about 60 mol % of a compound of any one of claim 1 to claim 6; about 2 mol % to about 25 mol % neutral lipid; about 18.5 mol % to about 60 mol % structural lipid; and about 0.2 mol % to about 10 mol % of PEGylated lipid.

35. The lipid nanoparticle of claim 29, wherein the lipid nanoparticle further comprises a polynucleotide.

36. The lipid nanoparticle of claim 35, wherein the polynucleotide is selected from the group consisting of: a messenger RNA (mRNA), a self-amplifying mRNA (sa-mRNA), a small interfering RNA (siRNA), a microRNA (miRNA), miRNA inhibitors (antagomirs/antimirs), messenger-RNA-interfering complementary RNA (micRNA), short hairpin RNA (shRNA), multivalent RNA, dicer substrate RNA, an antisense oligonucleotide, plasmid DNA, DNA, and complementary DNA (cDNA).

37. The lipid nanoparticle of claim 35, wherein the polynucleotide is a ribonucleic acid (RNA).

38. The lipid nanoparticle of claim 35, wherein the polynucleotide is a conventional or self-amplifying mRNA.

39. The lipid nanoparticle of claim 29, wherein the lipid nanoparticle has a diameter of from about 30 nm to about 160 nm.

40. A pharmaceutical composition comprising a plurality of lipid nanoparticles of claim 29, and a pharmaceutically acceptable carrier.

41. A method of delivering a polynucleotide to a mammalian cell, including administering the lipid nanoparticle of claim 29, or the pharmaceutical composition of claim 40, to a subject to thereby contact the cell with the lipid nanoparticle or pharmaceutical composition and deliver the polynucleotide to the cell.

42. The method of claim 41 wherein the cell is a cell of a human subject.

43. A method of producing a polypeptide of interest in a mammalian cell, including the step of contacting the cell with a lipid nanoparticle of claim 29, or the pharmaceutical composition of claim 40, wherein the lipid nanoparticle comprises a conventional mRNA or a self-amplifying mRNA encoding the polypeptide.

44. A method of treating a disease, disorder or condition in a subject in need of such treatment, comprising administering a lipid nanoparticle of claim 29, or the pharmaceutical composition of claim 40, to the subject to thereby treat the disease, disorder or condition.

45. The method of claim 44, wherein the disease, disorder or condition is selected from the group consisting of a rare disease, an infectious disease, cancer, a proliferative disease, a genetic disease, an autoimmune disease, diabetes, a neurodegenerative disease, a cardiovascular disease, a reno-vascular disease and a metabolic disease.

46. A vaccine comprising a lipid nanoparticle of claim 1, or the pharmaceutical composition of claim 40, and a mRNA encoding a polypeptide.

47. The vaccine of claim 46, wherein the vaccine is selected from a tumor vaccine, an influenza vaccine, and a SARS, including a SARS-CoV-2, vaccine.
